(19) United States
(12) Patent Application Publication
(10) Pub. No.: US 2026/0297611 A1
(43) Pub. Date: Oct. 1, 2026

(54) DEVELOPMENT OF GENETICS-BASED SELECTION FOR SPORELESS KELP

(71) Applicants: UNIVERSITY OF SOUTHERN CALIFORNIA, Los Angeles, CA (US); WOODS HOLE OCEANOGRAPHIC INSTITUTION, Woods Hole, MA (US); UWM RESEARCH FOUNDATION, INC., Milwaukee, WI (US); UNIVERSITY OF CONNECTICUT, Farmington, CT (US)

(72) Inventors: Scott LINDELL, Woods Hole, MA (US); Sergey NUZHDIN, Los Angeles, CA (US); Kelly DEWEESE, Los Angeles, CA (US); Gary MOLANO, Los Angeles, CA (US); Charles YARISH, Farmington, CT (US); Filipe ALBERTO, Milwaukee, WI (US)

(73) Assignees: UNIVERSITY OF SOUTHERN CALIFORNIA, Los Angeles, CA (US); WOODS HOLE OCEANOGRAPHIC INSTITUTION, Woods Hole, MA (US); UWM RESEARCH FOUNDATION, INC., Milwaukee, WI (US); UNIVERSITY OF CONNECTICUT, Farmington, CT (US)

(21) Appl. No.: 19/479,788

(22) PCT Filed: May 5, 2024
(86) PCT No.: PCT/US2024/027903

§ 371 Date: Oct. 29, 2025

Related U.S. Application Data
(60) Provisional application No. 63/463,978, filed on May 4, 2023.
(60) Provisional application No. 63/543,798, filed on Oct. 12, 2023.

(51) Int. Cl. C12N 15/82; A01G 33/00

(52) U.S. Cl. CPC C12N 15/8287; A01G 33/00

(57) ABSTRACT

Provided herein are strains of sugar kelp comprising one or more mutations in the gametophytes designed to produce infertile sporophytes.

### CROSS-REFERENCE TO RELATED APPLICATIONS

This application claims benefit under 35 U.S.C. § 119 (e) of U.S. Provisional Application Nos. 63/543,798 filed Oct. 12, 2023, and 63/463,978 filed May 4, 2023, the contents of which are incorporated herein by reference in their entirety.

### STATEMENT REGARDING FEDERALLY SPONSORED RESEARCH OR DEVELOPMENT

This invention was made with government support under Grant Nos. DE-AR0000914 and DE-AR0000915 awarded by the Department of Energy. The government has certain rights in the invention.

### TECHNICAL FIELD

The technology described herein relates to kelp aquaculture.

### BACKGROUND

Current kelp aquaculture, among other brown algae aquaculture, is a rapidly expanding global industry with the potential to disrupt the biofuel, food, pharmaceutical, carbon sequestration, and cosmeceutical markets. However, global kelp aquaculture is limited to farming native species in their native habitats which is reminiscent of early agriculture. While certain kelp species have been domesticated in China, Korea, and Japan, the overall scope of global kelp aquaculture is fractured when compared to the present state of agriculture. The “green revolution” in agriculture initiated a drastic reduction in the number of crop species being cultivated, coincided with a limited number of superior breeds of crops being produced and promoted technological advances to further boost agricultural production. (Kim et al. 2017. Seaweed aquaculture: cultivation technologies, challenges and its ecosystem services. Algae 32 (1): 1-13; Hwang E. K. et al. 2022. Kelps in Korea: from population structure to aquaculture to potential carbon sequestration. Algae 37 (2): 85-103; and Kim et al. 2019. Opportunities, challenges and future directions of open water seaweed aquaculture in the United States. Phycologia 58 (5): 446-461).

However, an alternative solution to the “green revolution” is required as the amount of available arable land available is rapidly shrinking as the world population increases towards its carrying capacity. A parallel “blue revolution” for kelp aquaculture across the vast oceans is that new solution, in which the number of cultivated kelp species is reduced and/or kelp cultivars are increased, superior breeds of kelp are identified, and technology is developed to farm kelp across various ocean conditions to the levels required to greatly impact the global economy. However, numerous issues hinder this start of the “blue revolution” (Hwang et al. 2022).

First and foremost, valid concerns regarding the effects of invasive species, genetic introgression (i.e., transmission of less diverse domesticated genotypes into wild kelp populations), and/or transgene flow (i.e., transmittance of genes to subsequent generations as a part of evolution) on the environment, ocean ecosystems, and natural populations of kelp currently impede the expansion of kelp aquaculture. These ecological concerns arose after “green revolution” and did not prevent staple crops, such as wheat, from being continually cultivated outside of their native habitats, regardless of their environmental effects.

Today, regulatory bodies are much more concerned about the environmental consequences of cultivating non-native and domesticated crops and specifically in areas where a native population is present, and this issue is especially magnified in ocean farming. Mistakes made locally in ocean aquaculture can be quickly exacerbated by ocean currents. Since many nations and billions of people rely directly on the ocean for sustenance, any potential negative effect on the ocean ecosystem must be accounted for. Kelp is a primary producer, and disturbances to local kelp populations may have devastating effects on local ecosystems, and these effects would be further compounded up the food chain. Therefore, overcoming precautionary principles and/or obtaining permits for kelp aquaculture is difficult, and these matters must be addressed before ideal species and superior breeds of kelp are farmed on a large scale.

Another main impediment preventing kelp from having a greater impact on the global economy is the limited scale of production: currently, kelp is principally cultivated in its native coastal habitats instead of across the entire ocean and/or in open ocean environments. While two kelps, giant kelp (Macrocystis pyrifera) and sugar kelp (Saccharina latissima), are being cultivated in the United States, the impact on the economy is muted by limited farm space and permitting concerns. The open ocean is a tantalizing target for the expansion of kelp aquaculture as oceans cover over 70% of Earth and this space is grossly underutilized. For example, open ocean kelp aquaculture focused on biofuel production would be preferable to corn or soybean biofuels as kelp can harness nutrients that are naturally abundant and available in the ocean, eliminating the need for fertilizers and freshwater, and freeing up arable land for food production. A solution is needed to safely cultivate kelp in the open ocean before aquaculture can reach its economic potential.

A portion of current kelp industry, e.g., most of the current US kelp industry, involves harvesting “wild” kelp from naturally occurring beds (Kim et al. 2019). While gathering freely available kelp is an efficient way to produce some crop yield, cultivating and maintaining superior breeds of kelp will maximize the yield and economic potential of kelp aquaculture. One strength of the “green revolution” was developing phenotypic consistency for crops; that is, specific crops would develop at the same rate, grow to the same height, produce a similar amount of fruit/biomass. Phenotypic consistency is advantageous for farmers because it eases the logistical challenge regarding planting, harvesting, and maintenance of farms and maximizes yields. In order cultivate kelp on large scale farms in the ocean, there is a need for phenotypically consistent kelp, just as in wheat, corn, and rice.

Additionally, some State regulations restrict the planting of select strains even if very they are recently from the wild. Whether the seed origins are selectively bred or not, some States exert a high bar and precautionary principles to keep kelp seed and farms as similar as possible to their wild cousins, thereby narrowly constraining what can be planted commercially. Genetic selection in terrestrial agriculture and marine aquaculture has a track record of productivity improvement of ~10% per generation (Gjedrem et al., 2012), and it is feasible that the yield of seaweed per hectare could double within 10 years by applying classic selective breeding methods alone. Historically, the kelp farming industry has relied on wild collected kelp spores of undocumented growth performance and composition of which some strains or closely related species or sub-species, like Saccharina latissima forma angustissima (also known as skinny kelp) have been overharvested and are now threatened (Augyte et al. 2017 Cultivation of a morphologically distinct strain of the sugar kelp, Saccharina latissima forma angustissima, from coastal Maine, USA, with implications for ecosystem services J Appl. Phycol. 29 (4): 1967-1976; and Augyte et al., 2018. Speciation in the exposed intertidal: the case of Saccharina angustissima comb. nov. & stat. nov. (Laminariales, Phaeophyceae). Phycologia 57 (1): 100-112., 2018). Additionally, this species is often farmed and harvested as a sustainable food product. Currently, there are no methods, e.g., no commercial adoption of methods, that allow for the consistent and reliable kelp seed supply and performance (e.g., US kelp seed supply and performance). While methods of plant breeding have been applied to Saccharina japonica (Zhang et al., 2007, 2011; Li et al., 2007), S. latissima performance has only been improved very recently by the present inventors. Improvement in yield has been in excess of 10% per generation with some select crosses more than twice typical commercial yields. Relative to flowering plants, kelp has an attribute unexploited for breeding yet: its biphasic life history (FIG. 1). Thus, the use of kelp strains that are selected for better performance, but incapable of reproducing would be advantageous particularly in kelp farm areas adjacent to wild kelps strains.

The haploid gametophyte stage of kelp is easily cloned (grown vegetatively), maintained, and sequenced. In the era of genomic assisted breeding, DNA markers enable the prediction of the breeding value of gametophytes, e.g., for different traits. Furthermore, statistical methods can be devised to predict the combining ability of gametophytes to better predict the performance of sporophytic progeny of two gametophytes, such that high-yielding sporophytes can be designed. Cloning the gametophytes allows practically unlimited quantities of genetically uniform sporophytes to be propagated from the crossing of a male and female clone.

Improved strains (fertile and infertile (e.g., non-reproductive)) can be created and selected for a broad assortment of traits and attributes such as ash-free dry weight yield and total sugar content to maximize available energy. One can imagine a bio-refinery approach where different fractions are extracted at different stages to maximize revenue that include proteins, lipids, bio-active compounds, etc. in addition to carbohydrate fractions.

Thus, any “improved” kelp developed through domestication of “superior” species and/or cultivars of kelp will have difficulty obtaining permits globally due to fears of invasive species, trans gene flow between natural populations, and potential impacts of escape from kelp farms into local ecosystems. The present invention relates to a solution to these issues by directly addressing concerns limiting kelp aquaculture and clearing the way for the farming of superior but infertile breeds of kelp in non-native habitats and the open ocean.

### SUMMARY

The present disclosure describes compositions and methods relating to improved strains of brown algae (Phaeophyceae class) such as sugar kelp (Saccharina latissima), giant kelp (Macrocystis pyrifera), among other species of seaweeds in the order Laminariales with the capabilities to control its reproduction. Specifically, this disclosure includes kelp gametophytes comprising one or more mutations, e.g., as detailed in Tables 3-7, which when crossed produce infertile or sterile (e.g, non-reproductive) sporophytes. When these sporophytes are seeded, the resulting mature kelp is unable to further create gametophytes, e.g, viable gametophytes, via meiosis.

Although the methods used herein may be achieved by known breeding techniques, the use of modern genomic tools including, but not limited to, targeted gene editing, ploidy manipulation, mutation generation, UV irradiation, etc. is also applicable.

This invention discloses (a) kelp germplasm (parental gametophytes) that are at least one generation from the wild (F1); (b) a matching set of genetic sequences of this F1 collection so as to create genetic markers that can facilitate further breeding improvement via algorithms that can predict performance; and (c) the identification of parental germplasm (male and female gametophytes pairs) that produce infertile (e.g., non-reproductive) sporophytes. As used here, “F1” refers to the haploid gametophyte stage (e.g., as kept in a seed bank) and continues to be the F1 without regard to vegetative propagation of said haploids, and “F2” refers to the diploid sporophyte stage resulting from the crossing of the F1 gametophytes. This invention discloses (a) kelp germplasm (parental gametophytes); (b) a matching set of genetic sequences of this collection so as to create genetic markers that can facilitate further breeding improvement via algorithms that can predict performance; and (c) the identification of parental germplasm (male and female gametophytes pairs) that produce infertile (e.g., non-reproductive) sporophytes.

### BRIEF DESCRIPTION OF THE DRAWINGS

The drawings described herein constitute part of this specification and includes exemplary embodiments of the present invention which may be embodied in various forms. It is to be understood that in some instances, various aspects of the technology described herein may be shown exaggerated or enlarged to facilitate an understanding of the technology described herein. Therefore, drawings may not be to scale.

FIG. 1 depicts the biphasic life cycle of kelp. Diploid kelp, the sporophyte, grows to be a large producer of biomass. Its reproductive tissue (the sorus) generates meiospores which germinate to become haploid male and female gametophytes. These are easily stored, cloned, maintained, and sequenced. Gametophytes in turn produce eggs and sperm which unite to form diploid zygotes that develop into SPs. Image is from Redmond et al. 2014.

FIG. 2 depicts the haplodiplontic life cycle of a brown macroalgae. The oogonium (female) and the antheridia (male) are the gendered name of gametophytes.

FIG. 3 depicts an embodiment of the procedural plan to generate sterile (e.g., non-reproductive) gametophytes using natural allele selection.

FIG. 4 depicts one embodiment of creating infertile or sporeless kelp strains wherein female and male gametophytes are selected which comprise one or more mutations in recessive sterile (e.g., non-reproductive) alleles in genes required for meiosis that prevent the developing sporophyte from producing meiospores, e.g., viable meiospores.

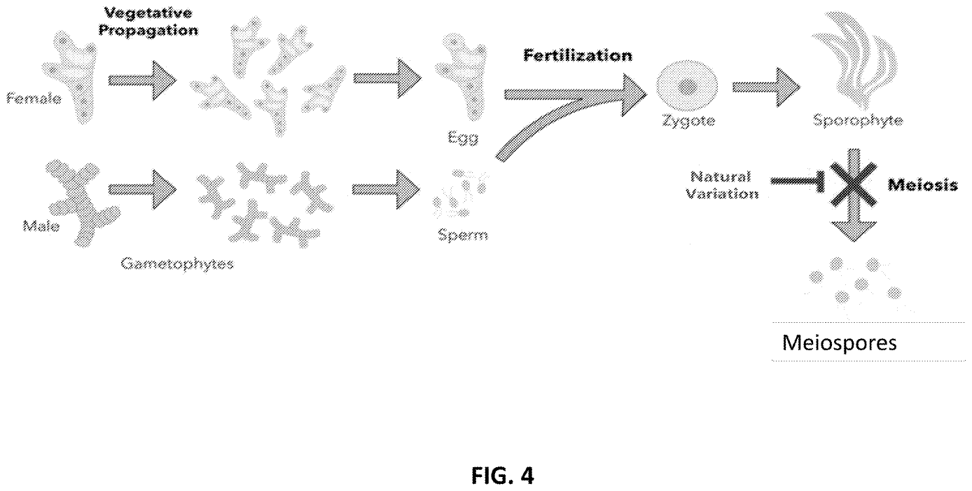

### DETAILED DESCRIPTION

The technology described seeks to address a primary challenge in the aquaculture industry of how to expand kelp cultivation greatly and responsibly for biofuel, food, pharmaceutical, carbon sequestration, and cosmeceutical applications by developing breeds of sterile (e.g., non-reproductive) kelp. Sterile (e.g., non-reproductive) kelp would address major concerns regarding permitting the farming of non-native species and/or domesticated cultivars of kelp and potential trans gene flow into native populations of kelp. Without sterile (e.g., non-reproductive) kelp, improved breeds of kelp currently being developed might not be grown at all, or could only be grown in their native habitats, which are crowded coastal areas, preventing the mass cultivation of kelp required to disrupt the energy market. Giant kelp (Macrocystis pyrifera) and sugar kelp (Saccharina latissima), two species of brown macroalgae with great farming potential in the U.S., have been selected as targets for the development of sterile (e.g., non-reproductive) kelp breeds for out-planting in the open ocean. However, as the genetic targets discussed herein are highly conserved, other species of seaweeds and kelp may also be adaptable to the methods of the present disclosure.

Haploid kelp gametophytes with mutations in genes specific and essential for reproduction can still vegetatively propagate, and can be produced by leveraging the two-stage (biphasic) life cycle of brown macroalgae. The technique involves using genetics and/or genomics-based selection to find these gametophytes that are produced from wild kelp. These gametophytes can be vegetatively propagated en masse in culture and reliably fused to generate viable, sterile (e.g., non-reproductive) adults. Targeting genes that are only expressed in the adult sporophyte reproductive tissue will ensure that mutations in these genes will have no effect on the growth and development of a healthy kelp from gametophyte to sporophyte but will prevent the sporophytes from producing a next generation of spores (e.g., meiospores, sometimes called zoospores (see, e.g., Redmond, S. et al. 2014. “New England Seaweed Culture Handbook” Seaweed Cultivation. Paper 1, pg. 20)) rendering them completely sterile (e.g., non-reproductive). Since gametophytes can be vegetatively propagated in culture, gametophytes with ideal mutations can be maintained indefinitely, serving as a germplasm bank to match gametophytes of the opposite sex with sterility mutations in same genes. Thus, healthy gametophytes will produce sterile (e.g., non-reproductive) sporophytes, with identical genotypes able to be produced year after year. Phenotypic consistency is advantageous for farmers because it eases the logistical challenge regarding planting, harvesting, and maintenance of farms and maximizes yields. Sterile (e.g., non-reproductive) kelps will then be evaluated for growth in ocean conditions. Another potential benefit of sterile (e.g., non-reproductive) kelp breeding to be investigated is additional biomass production, as resources normally spent on reproduction could instead be channeled towards growth.

The proposed development of sterile (e.g., non-reproductive) kelp breeds for two key energy-production species in the US would provide for the foundation of a “blue revolution” and is key for the expansion of kelp cultivation to un- or under-utilized open ocean space. By generating a seed bank of gametophytes with sporophyte sterility (e.g., non-reproductive) knockout mutations, e.g., through bioinformatic analysis, this invention addresses fundamental challenges in aquaculture ranging from invasive species, transgene flow, aquaculture permitting, cultivation scale, and phenotypic consistency.

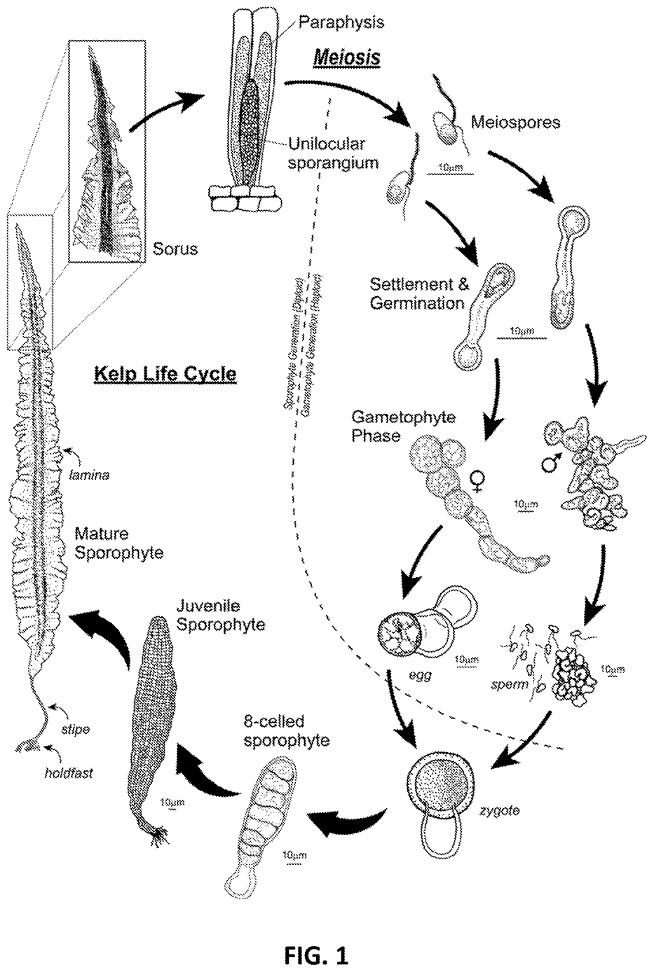

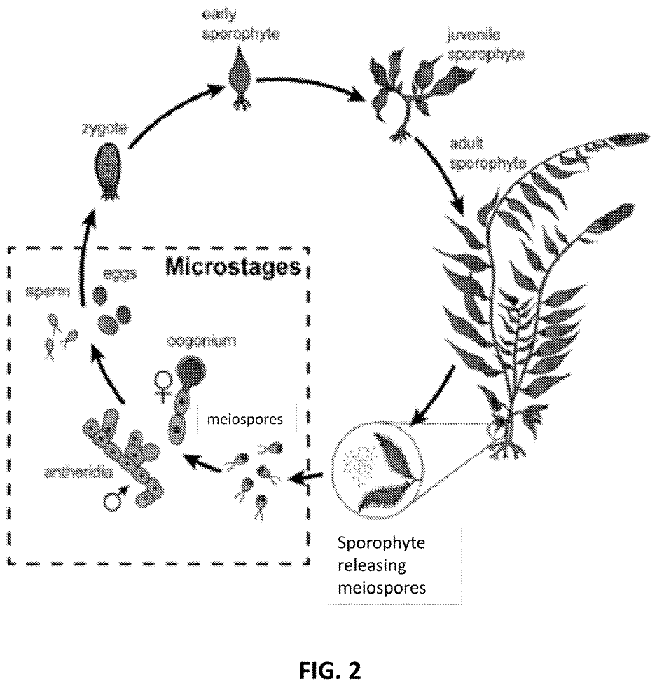

Brown macroalgae, or kelp, have a haplo-diplontic life cycle that can be manipulated for aquacultural purposes (examples of the sugar kelp and giant kelp life cycles are shown in FIGS. 1 and 2). The adult kelp, or sporophyte, is a diploid macroalga that grows quickly and can reach over a hundred feet in length, e.g., in some species. After the kelp has reached maturity, sporophylls, or leaves containing reproductive tissue full of haploid meiospores, develop on the blades of the sporophytes. The haploid meiospores are then subsequently released and settle, developing into male and female haploid gametophytes. Male gametophytes then produce sperm, while female gametophytes produce eggs and/or hormones that stimulate the males. The males then release the sperm, which find and fertilize the female eggs, e.g., via hormonal sex attractants, creating a diploid young or juvenile sporophyte. Gametophytes can be continuously vegetatively propagated for extended periods of time under laboratory conditions.

One embodiment described herein aims to generate brown macroalgae gametophytes with loss-of-function, e.g, knockout, mutations in the genes required for sporophyte fertility to reliably produce sterile (e.g., non-reproductive) sporophytes (adults) for applications in aquaculture. In particular, this embodiment described herein seeks to leverage the haplodiplontic life cycle of brown macroalgae in two ways: 1) exploit the differential expression between the gametophyte and sporophyte life cycles to identify genes essential and specific for reproduction that do not affect normal development of gametophytes and sporophytes 2) utilize gametophyte vegetative propagation in culture to mass produce identical genotypes with the necessary mutations in fertility genes to attain sterile (e.g., non-reproductive) adults. This embodiment is applicable to the expansion of brown macroalgae aquaculture into non-native habitats such as the open ocean, addresses ecological concerns relating to invasive species and trans gene flow, eases the permitting process for aquaculture, and increases the repeatability of preferred, consistent phenotypes.

Another embodiment described herein relates to a novel technique of generating sterile (e.g., non-reproductive) kelp through genomic-based selection, genetics-based selection, and/or mutagenesis methods. Another embodiment described herein generates a bank of “immortal” haploid gametophytes that have mutations in genes essential for successful reproduction that can be reliably vegetatively propagated in culture and fused with a matching mutant gametophyte to generate sterile (e.g., non-reproductive), diploid sporophytes. The mutant genes should have no effect on the propagation of the gametophytes or the development of the adult, as the candidate mutant genes should have their expression restricted to the maturation of reproductive tissues, e.g., in the diploid sporophyte and/or the development of viable, haploid spores, e.g., meiospores. Verified “sterile” gametophytes can be vegetatively propagated in mass, producing a plethora of identical “sterile” gametophytes to satisfy the predicted demand of kelp farmers.

A further embodiment described herein is the establishment of a sterility (e.g., non-reproductive) candidate gene bank. The candidate genes expression should be essential and specific for the formation of viable spores, e.g., meiospores and thus should not affect the viability, fertilization, development, or vegetative propagation of the gametophytes, or the development of the sporophyte. One cellular process for knockout targeting is meiosis. Unlike mitosis, which is essential for the development of both the gametophyte and the sporophyte, the meiotic pathway is only active in reproductive tissues, as the kelp life history shifts from diploid to haploid phases. However, the ideal mutations would prevent the formation of the reproductive tissues in the sporophyte altogether. Differential tissue expression analysis on the kelp transcriptome can be performed in order to find more candidate sterility genes. The different tissues to focus on would be gametophytes, young sporophytes, sporophyte reproductive tissue, sporophyte non-reproductive tissue, and spores, e.g., meiospores. Genes that are only expressed in the reproductive tissues would be ideal sterility candidates. A sterility gene is a gene that, when completely knocked out or knocked down, in a sporophyte, causes sterility in that sporophyte.

Another embodiment described herein involves methods of sterilizing gametophytes. Gametophytes with sterility mutations that can be vegetatively propagated en masse in culture, and when fertilized, produce sterile (e.g., non-reproductive) kelp adults. The following methods are embodiments described herein. The different routes to sterile (e.g., non-reproductive) kelp detailed here include using combinations of naturally occurring alleles and mutation breeding. Once male and female gametophytes with sterility (e.g., non-reproductive) mutations in the same genes have been experimentally identified, the gametophytes will be fused, and the adult will be carefully monitored for the development of viable spores, e.g., meiospores to test the validity of the sterile (e.g., non-reproductive) sporophytes. An advantage of the technology described herein is that once a gametophyte with a mutation in a sterility gene is identified, clones of that gametophyte (and/or clones thereof) can then be vegetatively propagated indefinitely in the laboratory, providing a stable supply of identical “sterile” gametophytes than can be used as a base for expanding kelp aquaculture for years without needing to make new “sterile” gametophytes.

Once the candidate sterility (e.g., non-reproductive) gene bank has been established, specific mutations found as naturally occurring alleles or mutagenized using ionizing radiation. One embodiment described herein is a frame shift mutation at the beginning of an essential sterility gene. Frame shift mutations are more difficult for organisms to repair in comparison with point mutations or nonsense mutations because they shift the codon sequence, thus causing mistakes that are translated downstream of the mutation.

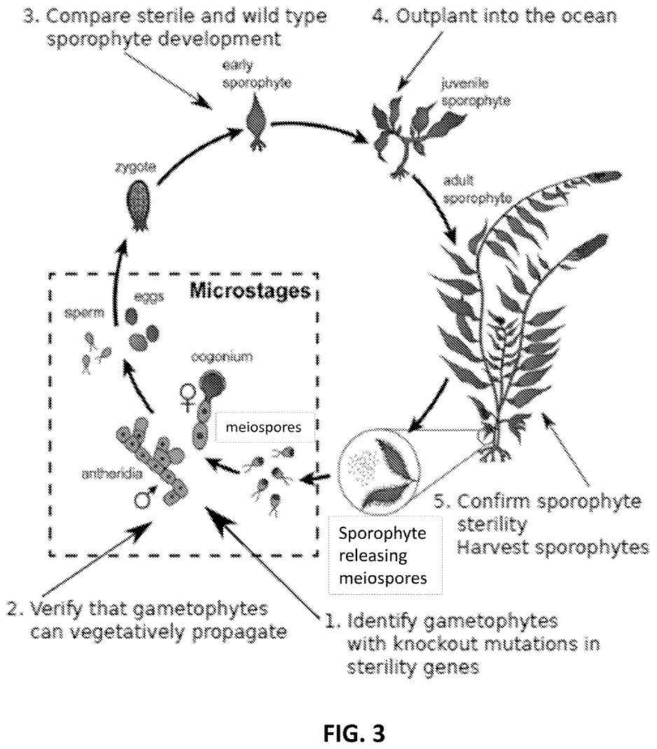

Another embodiment described herein involves identifying gametophytes that carry natural mutations in sterility (e.g., non-reproductive) genes (FIG. 3). This population genetics-based approach takes advantage of the haplodiplontic life cycle of brown macroalgae. Genes that are essential for haploid gametophyte viability and development are under intense purifying selection; since gametophytes only have one copy of a gene, any mutation that disrupts gene expression and activity essential in gametophytes will be selected against and removed from the population. However, genes that are only essential in the diploid sporophyte stage do not undergo this intense purifying selection.

Gametophytes can have mutations that disrupt gene function in the sporophyte stage can produce viable, fertile sporophytes by fusing with a gametophyte of the opposite sex that carries a wild-type version of the specific sporophyte gene. Population genetics dictates that there is a standing variation in sporophyte genes in natural populations of kelp, including in genes that are essential for sporophyte fertility. By surveying gametophytes from natural populations of kelp, gametophytes with frame shift or knockout mutations in genes previously identified as candidate sterility genes can be identified. These candidate sterile (e.g., non-reproductive) gametophytes can then be vegetatively propagated in culture and crossed with other candidate sterile (e.g., non-reproductive) gametophytes to assess effects on sporophyte fertility. Sterile (e.g., non-reproductive) adult kelps can be produced without genetic engineering by pairing sterile (e.g., non-reproductive) gametophytes that carry mutations found in natural kelp populations in matching sterility (e.g., non-reproductive) genes. Some of these candidates have been found in the genes Spo11, Mre11, HFM1, and Rad50.

In a specific embodiment, gametophytes are identified through a bioinformatic selection process. After the sequencing of the sugar kelp gametophyte cultures, raw reads were trimmed and aligned to the draft sugar kelp genome. Polymorphisms were then called and filtered for quality control. Mutations were then classified, and potential candidate mutations were selected based on high effect (i.e., nonsense and frameshift classification). Genes were predicted to be candidate seedless genes based on subsequent filtering: Blastp of annotated Ectocarpus siliculosus and Macrocystis pyrifera meiotic genes, JGI annotated meiotic genes, and genes differentially expressed in sugar kelp sorus tissue versus other sugar kelp tissue types. Candidate crosses were then designed with complementary high effect mutations to confer the sporeless phenotype.

Another embodiment described herein is mutation breeding. Mutation breeding has been used in plants to produce phenotypes that are either rare or nonexistent in natural populations. Mutation breeding for sterile (e.g., non-reproductive) kelp would involve exposing gametophytes to mutagenizing chemicals or ionizing radiation to produce mutants in the sterility genes. Gametophytes that still could vegetatively reproduce in culture could then be checked for mutations in sterility genes.

Another embodiment described herein is to generate a seed bank of “sterile” gametophytes that produce sterile (e.g., non-reproductive) sporophytes can then be readily sold to kelp farmers. Seed banks are one of several breeding systems used in current agricultural breeding programs to generate hybrid plants that outperform homozygous or autozygous lines. One principle of modern plant breeding is to produce plants with sterile male inflorescences as the F0 generation. Sterile male inflorescences eliminate the possibility of selfing and thus requires external fertilization, ensuring production of hybrid F1 seeds that are then sold to farmers. Current seed companies are using these elaborate but overly complex practices to produce hybrid seeds because these stocks cannot be propagated vegetatively at the haploid state and thus must be generated year after year. Our innovation takes advantage of the ability of brown macroalgae to vegetatively propagate in their gametophyte, or seed, stage, and thus eliminates many of the problems associated with generating hybrid seeds seen in current agriculture.

An additional benefit to a sterile (e.g., non-reproductive) kelp seed bank is phenotypic consistency. In agriculture, many generations of inbreeding and double haploid production aided in creating phenotypically consistent crops by reducing the heterozygosity of the crop genomes. Phenotypic consistency can be achieved much faster in kelp, as gametophytes can be vegetatively propagated in laboratory conditions, producing identical, immortal, gametophytes that can then be fused en masse to produce adults with identical genotypes. Additionally, various crosses that optimize phenotypes for specific markets, such as increasing the biomass produced by an individual plant, can be easily reproduced due to kelp gametophytes' vegetative propagation capability. While there are other methods to make the sterility mutations, the main procedures to generate the invention have been mentioned above. It is important to note that the invention covers generating gametophytes that can be constantly vegetatively propagated, contains knockout mutations in genes essential for fertility, and reliably produces sterile (e.g., non-reproductive) sporophytes when the gametophytes are fused. While the paths to achieve kelp sterility are important, the novel invention and end goal is producing a bank of “sterile” gametophytes that establishes a foundation to greatly expand kelp aquaculture.

Deep sequencing of the sugar kelps strains has identified a plurality of mutations that when gametophytes comprising one or more of these mutations are combined, the resulting sporophyte is unable to create its own gametophytes and therefore unable to reproduce (Tables 1-7). As such, these mutated strains may be seeded without restrictions and have no effect on wild strains.

Table 1 provides a listing of multiple sugar kelp strains of which have been engaged to create F2 generation kelp with the inability to reproduce. As used here, “F1” refers to the haploid gametophyte stage (e.g., as kept in a seed bank), and “F2” refers to the diploid sporophyte stage resulting from the crossing of the F1 gametophytes.

Described in Table 2 is a summary of strain crosses which have resulted in infertile sporophytes.

As detailed in Tables 3-7, a plurality of mutations has been identified. Such mutations alone or in combination may result in mutated gene products that are involved in sporophyte generation.

In one aspect of any of the embodiments, described herein is an algal gametophyte comprising one or more mutations of Spo11, Mre11, Rad50, HFM1, SAMSARA, OUROBOROS, and Tables 3-7.

In some embodiments of any of the aspects, the algal gametophyte is an isolated algal gametophyte. In some embodiments of any of the aspects, the brown algal gametophyte is an isolated algal gametophyte. In some embodiments of any of the aspects, the isolated algal gametophyte is isolated from algal gametophytes of the opposite gender. In some embodiments of any of the aspects, the isolated algal gametophyte is isolated from non-syngenic algal gametophytes of the same gender. In some embodiments of any of the aspects, the isolated algal gametophyte is isolated from algal gametophytes of the opposite gender and is isolated from non-syngenic algal gametophytes of the same gender.

In one aspect of the embodiments, described herein is a combination of algal gametophytes comprising: 1) a first algal gametophyte comprising at least a first mutation selected from: a mutation of Spo11, Mre11, Rad50, HFM1, SAMSARA, OUROBOROS, and Tables 3-7; and 2) a second algal gametophyte comprising at least a second mutation selected from: a mutation of Spo11, Mre11, Rad50, HFM1, SAMSARA, OUROBOROS, and/or Tables 3-7.

In one aspect of the embodiments, described herein is a combination of algal gametophytes comprising: 1) a first algal gametophyte comprising at least a first mutation selected from: a mutation of Spo11, Mre11, Rad50, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof; and 2) a second algal gametophyte comprising at least a second mutation selected from: a mutation of Spo11, Mre11, Rad50, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof.

In one aspect of any of the embodiments, described herein is an algal sporophyte comprising: at least a first mutation selected from: a mutation of Spo11, Mre11, Rad50, HFM1, SAMSARA, OUROBOROS, and/or Tables 3-7; and at least a second mutation selected from: a mutation of Spo11, Mre11, Rad50, HFM1, SAMSARA, OUROBOROS, and/or Tables 3-7. In one aspect of any of the embodiments, described herein is an algal sporophyte comprising: at least a first mutation selected from: a mutation of Spo11, Mre11, Rad50, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof; and at least a second mutation selected from: a mutation of Spo11, Mre11, Rad50, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof. In some embodiments of any of the aspects, the at least a first mutation is found on a first chromosome or first set of chromosomes, and the at least a second mutation is found on a second chromosome or second set of chromosomes, e.g., the at least a first mutation was inherited from one gametophyte ancestor and the at least a second mutation was inherited from the second gametophyte ancestor. In some embodiments of any of the aspects, the at least a first mutation is found on a first chromosome and the at least a second mutation is found on a second chromosome. In some embodiments of any of the aspects, the at least a first mutation and the at least a second mutation are not found on the same chromosome.

In one aspect of any of the embodiments, described herein is an algal sporophyte comprising: on a first chromosome, at least a first mutation in a gene selected from: Spo11, Mre11, Rad50, HFM1, SAMSARA, and OUROBOROS; and in a second chromosome, at least a second mutation in the selected gene. In one aspect of any of the embodiments, described herein is an algal sporophyte comprising: on a first chromosome, at least a first mutation in a gene selected from: Spo11, Mre11, Rad50, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof; and in a second chromosome, at least a second mutation in the selected gene. In one aspect of any of the embodiments, described herein is an algal sporophyte comprising: on a first chromosome (or first set of chromosomes) at least a first mutation in each of two genes selected from: Spo11, Mre11, Rad50, HFM1, SAMSARA, and OUROBOROS; and in a second chromosome (or second set of chromosomes), at least a second mutation in each of the at least two selected genes. In one aspect of any of the embodiments, described herein is an algal sporophyte comprising: on a first chromosome (or first set of chromosomes) at least a first mutation in each of two genes selected from: Spo11, Mre11, Rad50, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof; and in a second chromosome (or second set of chromosomes), at least a second mutation in each of the at least two selected genes. In some embodiments of any of the aspects, the at least two selected genes are Rad50 and Spo11. In some embodiments of any of the aspects, the at least a first mutation and the at least a second mutation are different mutations.

In some embodiments of any of the aspects, the algal sporophyte is an isolated algal sporophyte. In some embodiments of any of the aspects, the isolated algal gametophyte is isolated from non-syngenic algal sporophytes.

In one aspect of any of the embodiments, described herein is a composition or combination comprising: a) a first algal gametophyte comprising at least a first mutation in a gene selected from: Spo11, Mre11, Rad50, HFM1, SAMSARA, and OUROBOROS; and b) a second algal gametophyte comprising at least a second mutation in the selected gene. In one aspect of any of the embodiments, described herein is a composition or combination comprising: a) a first algal gametophyte comprising at least a first mutation in each of two genes selected from: Spo11, Mre11, Rad50, HFM1, SAMSARA, and OUROBOROS; and b) a second algal gametophyte comprising at least a second mutation in each of the at least two selected genes. In one aspect of any of the embodiments, described herein is a composition or combination comprising: a) a first algal gametophyte comprising at least a first mutation in a gene selected from: Spo11, Mre11, Rad50, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof; and b) a second algal gametophyte comprising at least a second mutation in the selected gene. In one aspect of any of the embodiments, described herein is a composition or combination comprising: a) a first algal gametophyte comprising at least a first mutation in each of two genes selected from: Spo11, Mre11, Rad50, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof; and b) a second algal gametophyte comprising at least a second mutation in each of the at least two selected genes. In some embodiments of any of the aspects, the at least two selected genes are Rad50 and Spo11. In some embodiments of any of the aspects, the at least a first mutation and the at least a second mutation are different mutations.

As used herein, “gametophyte” refers to a haploid filamentous thallus (either male or female) that is capable of fusing with a second gametophyte of a second gender to form a diploid cell, or to or a form of the organism comprising only haploid cells. Algal gametophytes are also capable of vegetative propagation. Algal gametophytes can be male gametophytes or female gametophytes.

As used herein “sporophyte” refers to diploid multicellular thalli (or a form of the organism comprising such diploid cells). A wild-type sporophyte is also sometimes distinguished in the art by the ability to form spores (e.g., meiospores), e.g., flagellated meiospores, which develop into gametophytes. Some embodiments of algal sporophytes described herein are not able to form meiospores, e.g, they are infertile or sterile (e.g., non-reproductive). Accordingly, “sporophyte” as used herein refers to a lifecycle stage and not an intact ability to give rise to meiospores.

In some embodiments of any of the aspects, an algal sporophyte described herein is infertile. In some embodiments of any of the aspects, an algal sporophyte described herein is sterile (e.g., non-reproductive). In some embodiments of any of the aspects, an algal sporophyte described herein is meiosis defective. In some embodiments of any of the aspects, an algal sporophyte described herein does not form meiospores. In some embodiments of any of the aspects, an algal sporophyte described herein forms meiospores that cannot develop into gametophytes. In some embodiments of any of the aspects, an algal sporophyte described herein cannot release functional meiospores. As used herein, “Spo11” or “Sporulation-Deficient 11” (also known as TOPVIA) is the catalytic subunit of the meiotic double strand break transesterase, which forms DNA breaks during meiotic recombination. Sequences for algal Spo11 are known in the art, e.g., the Macrocystis pyrifera Spo11 protein, transcript, and genome sequences are available, respectively, under Protein Id: 1162841; Transcript Id: 1163879 and Location: scaffold_10: 9460228-9465753 (−) in the JGI PhycoCosm database.

In some embodiments of any of the aspects, Spo11 is a gene encoding a transcript at least 80% identical to SEQ ID NO: 4. In some embodiments of any of the aspects, Spo11 is a gene encoding a transcript at least 85% identical to SEQ ID NO: 4. In some embodiments of any of the aspects, Spo11 is a gene encoding a transcript at least 90% identical to SEQ ID NO: 4. In some embodiments of any of the aspects, Spo11 is a gene encoding a transcript at least 95% identical to SEQ ID NO: 4. In some embodiments of any of the aspects, Spo11 is a naturally-occuring gene encoding a transcript at least 95% identical to SEQ ID NO: 4. In some embodiments of any of the aspects, Spo11 is a gene encoding a transcript with the sequence of SEQ ID NO: 4.

>jgi|SlaSLCT1FG3_1|2189955|estExt_Genewise1Plus.
C_9160004 proteinId=2189955 transcriptId=2191211
(Spo11)
SEQ ID NO: 4
ATGCCGGTGACCGTGCTCTCTTCGCCCCTGGACGTGCGATTGCATGACCG
AGAACGCGTTCTCGACAAGATCCAAGCGATCGTGGTAGACTGGCTGGACC
AGCTGGCCGGCGGGGATCTCCCAGTCTTCGAAGCAGCGGACGAGGATCAA
GAAGACGATGATATCGTGGGGGAGGAAGCTGACAACCTCCGAGAGGGGGC
AACTTTTCGGGCACGCATCCTCTGCGTATTGAGCTTCTCAACGAGACTCC
TGGAGACTCGTAAAACTGCCACGCAAAGAGAGATTTACTACACCTACGTC
AAGCACTTTGCGAACCAGACCAAATGCAACGAGGCCATACTGGACTGCTG
CGCGTTGCTGGGTGAACCCCGGCATTGCCTCGGGATAGTGGCAAGCCCAA
GGGGGTACCTTGCAGGGCGCATCTCTCTTCGGGGGCAGGGATTCGAAGAC
TGGCAAGATCCCAGGTCGACGGGAGGAACATCAGGCCTCGCCATCACGGC
AGACTGGCTGACTCGCCCCCTCGAGTTGAAGTCCGACGCCCGCTACGTGG
TGGTCGTCGAGAAGGATGGCGTGTTCAATCGCATGGTGGAGGACGGATTT
TTCGACAGGCAAGCATATACAAAAACACACCCCCTCGGTCGATTGATACC
TTCGATTCTCGTGACCGGTCAGGGCTTTCCTCCGCTGGCCGTACGCGCCT
GCGTTCACAGGCTGGCTGAGGCCTTCGATCTGCCCGTTCTTGGCCTCGTC
GATTGCAACCCATTTGGACTGAGCGTTTTTCTTCTGTACAAGGAAGGCTC
GTCTCGGCAGGGCCGGGGTTCTGTGAAGTATGCAGTTCCAGGAATGAAGT
GGGTAGGCCTCCGCCCGTCCCAGCTCGAAGACTTGGACCTCCCCCACCAG
GTACACCAGGAACTTTCGTCTCTGGATATTGCCAGGGCGCGGTCGATCGC
AAATCTGCCAATCGTTTCCGAGAACCCCGACACATACGGGCAGGAGGTCG
CGTACTGGGCCGACGAAAACAACCCAGGCAAGCAAGTCAACAGGTCCGCG
TGCCCAGTCGGTGACTCGATGGCCGCTACTGCCCGAGCTTCAACATATCT
GCGATTCGTGGTTGAACAGGCAACGATCATGTGTTTTAGTGGCGGGCGAT
ACTCGTCTCGTTGGCAGGAAAAAATCTAG

As used herein, “Mre11” or “Meiotic Recombination 11” is an exonuclease that functions during meiotic recombination. Sequences for algal Mre11 are known in the art, e.g., the Macrocystis pyrifera Mre11 protein, transcript, and genome sequences are available, respectively, under Protein Id: 75886; Transcript Id: 75886; and Location: contig_45: 2218399-2226452 (+) in the JGI PhycoCosm database.

In some embodiments of any of the aspects, Mre11 is a gene encoding a transcript at least 80% identical to SEQ ID NO: 6. In some embodiments of any of the aspects, Mre11 is a gene encoding a transcript at least 85% identical to SEQ ID NO: 6. In some embodiments of any of the aspects, Mre11 is a gene encoding a transcript at least 90% identical to SEQ ID NO: 6. In some embodiments of any of the aspects, Mre11 is a gene encoding a transcript at least 95% identical to SEQ ID NO: 6. In some embodiments of any of the aspects, Mre11 is a naturally-occuring gene encoding a transcript at least 95% identical to SEQ ID NO: 6. In some embodiments of any of the aspects, Mre11 is a gene encoding a transcript with the sequence of SEQ ID NO: 6.

>jgi|Macpyr2|75886|CE75885_420 (MRE11)
SEQ ID NO: 6
ATGTCCGAGAATGAAAGCCTCAGTATCTTGGTGGCAACGGATAGCCATCT
GGGGTACATGGAGCGAGACCCCGTGAGAGGCAAGGACTCGTTCGCTGCTT
TCGAGGAGATGCTCCTCATCGCCAAGAAGAAGAAGGTGGACTTCGTTCTC
CTCGGAGGAGACCTGTTTCACGAGAACAAGCCCTCCCGGCGCACCCTGTA
CCGCACGATGGACATACTGCGTCGCCACTGCATGAGCGACGACCCTGTCG
GGTTCCAGATCATCAGCGAGCAGTCGCAGAACTTCAAGGACAGGTTCGGG
CACGTGAACTACGAGGACCCTTACTTCTCTGTGGGGCTGCCTGTCTTCAG
CATCCATGGCAACCATGACGATCCTACCCGGGAGGGAGGGGTGGAGGCGC
TCGCGGCGCTTGACCTGCTGCACGTGGCAAACCTCGTTAACTATTTCGGC
AAGAGTGACAAGGTAGATGATGTGGAAGTGAACCCCATCTTGATACAAAA
GGGGCTGACGAAGCTAGCTCTTTACGGAATGGGCAGCATGAGGGACGAGA
GGCTCAACCGAATGTGGCAGCAGAAGAAGGTGAGGTTTCTCCGCCCCCTG
GAGGATGACGGGGGCAAGGACTTCTTCAACGTCTTTGTGATTCACCAG

As used herein, “Rad50” or “Radiation Sensitive 50” forms a complex with Mre11 which binds to DNA breaks and catalyzes double strand break repair. Sequences for algal Rad50 are known in the art, e.g., the Macrocystis pyrifera Rad50 protein, transcript, and genome sequences are available, respectively, under the name fgenesh2_kg.13_#_6343_#_TRINITY_DN21354_c0_g1_i1; Protein ID: 9541934; and Location: scaffold_13: 6066130-6084723 in the JGI PhycoCosm database. Rad50 is conserved in the Ectocarpus genome.

In some embodiments of any of the aspects, Rad50 is a gene encoding a transcript at least 80% identical to SEQ ID NO: 3. In some embodiments of any of the aspects, Rad50 is a gene encoding a transcript at least 85% identical to SEQ ID NO: 3. In some embodiments of any of the aspects, Rad50 is a gene encoding a transcript at least 90% identical to SEQ ID NO: 3. In some embodiments of any of the aspects, Rad50 is a gene encoding a transcript at least 95% identical to SEQ ID NO: 3. In some embodiments of any of the aspects, Rad50 is a naturally-occuring gene encoding a transcript at least 95% identical to SEQ ID NO: 3. In some embodiments of any of the aspects, Rad50 is a gene encoding a transcript with the sequence of SEQ ID NO: 3.

>jgi|SlaSLCT1FG3_1|3391912|estExt_fgenesh1_pg.
C_4350012 proteinId=3391912 transcriptId=
3393168 (Rad50)
SEQ ID NO: 3
ATGATCGTCGGGGCAAATGGGTGCGGCAAGACGACCGTGATCGAATGCCT
CAAGTACGCCTGCACCGGGGCGCTCCCGCCCGGCGCGCGCAACGGCCATT
CCTTCGTCCACGACCCAAAGGTGTCGGGCACGAACGAGGTGAAGGCCAGC
ATCAAGCTCCGCTTCGCCGGCCGGGACGGCCGAGTGAGCGTGGTCATCCG
GTCGCTCCAGCTCACTCAGAAGCGGGTGTCTCTCGAGTTTAAGGCGCTCG
ACGGCGTAATCAGGACGAAGGATGACAACGGTGTGAGCGTGAGCATCAAC
CACAAGTGCACGGAGATGGACAAGCACGTGGATCTGCTGCAGGGAAAGAA
GCGATCCCTCGAGCTAACGTGCCGAGACCTTGGCGTGCGCATGGCAGAGA
AGAAAGAGAACATCGGCGATGCTCTAAGCACGGAGTCGGACGAGGCCCTC
GACAGGAACCTCAGGGAGTTCGACGCCAGGCTGGACACGTGGTCGGTCAG
ACGGTACACGTCAGAGGACATAGCGACCTTCGAGGAGGCGCTCCGCGGCG
CCCACGACAAGGCCCAGGCGGTGCTCAACGAGACTCGGGCCAGCACTCGG
CTGGAGGACGAGTCGATGAACGCGGAGATGAACACTCTTAAGCTGGAGCA
GAGGAGCAGCTCGGCCGCCGTGCACGCAAAGGAGCGCGAGCTGCAGAAGA
TTCAGGGTGAAGCCATGTCGTTGGCGCAGGGCGGAGGGTCTGTGGGTGCC
GGGAGCCAAAGCCAGCTGGGTACGGCGACAGTGTCCAAGCTGGAGCGGGA
GCTGAAGGCTGCGGAGAAGGCTAAGAAGCAGTGA

As used herein, “HFM1” or “Helicase for Meiosis 1” (also known as MER3 or RCK) is a DNA helicase. Sequences for algal HFM1 are known in the art, e.g., the Macrocystis pyrifera HFM1 protein, transcript, and genome sequences are available, respectively, under Protein ID: 3387767; and Location: scaffold_142: 164464-255094 in the JGI PhycoCosm database.

In some embodiments of any of the aspects, HFM1 is a gene encoding a transcript at least 80% identical to SEQ ID NO: 5. In some embodiments of any of the aspects, HFM1 is a gene encoding a transcript at least 85% identical to SEQ ID NO: 5. In some embodiments of any of the aspects, HFM1 is a gene encoding a transcript at least 90% identical to SEQ ID NO: 5. In some embodiments of any of the aspects, HFM1 is a gene encoding a transcript at least 95% identical to SEQ ID NO: 5. In some embodiments of any of the aspects, HFM1 is a naturally-occuring gene encoding a transcript at least 95% identical to SEQ ID NO: 5. In some embodiments of any of the aspects, HFM1 is a gene encoding a transcript with the sequence of SEQ ID NO: 5.

>jgi|SlaSLCT1FG3_1|3387767|estExt_fgenesh1_pg.
C_1420009 proteinId=3387767 transcriptId=
3389023 (HFM1)
SEQ ID NO: 5
ATGTTGTTTTTTTACCGCTTCCGCCAAGTGTCGTTACCGCCGAAAAATGAAAAACCGCCAACC
GCCAACCGCCAAAAAAAGACGCCGTACGTATTGCTTTACCGCCGAAAATATACCACCATATTT
TGGTTTTCCGCTTCCGCCAAAGTCGTTAACGCCGAAAACGAGAAAACCGCAAACCACCTAAAA
ATTACCACCGTATGGAAAAATACCGCCCCAACTGTGTCCGCTCGAAAAACCGTTACAGACGAC
CACGCTGGACGAAAAGGATACCGCCCAGCAGTCACGACCATCACGACGTCGCAGCCGCGATTG
AGTTCGGTACTGCGGTTGACCAATGCTCCAAACGCGCGTGCTCTGTTCGACCTTGACGCTGAG
ATACTCACGAGAGAGAGAGCTGAAGCGCAATGGAAGAGCCAAGCTGACGCGAAATATTCGGCG
CCCTTCACATGGAAGGCGTTTTCCGAGAAGGTCCCCGCGAGCGAGGCGCAGAGAGCTACGGTG
GAGCAAGACAAGTTTCGCAGGTACTCTAGACTGGCGGGGAAGCTAGCGGGAGGCGACGACGGG
GCGGCGTTTCCCAACGCGGACCGGGCGCTCTTCCAGGCGATCAACTCGGCGAAGGGTGCGGCG
GAACAGGCCAAGAGGGACCTTGAGCTCGTGGTGGGAAAAGTGCCTGCCAAAGACTGGGCCGAG
GCCACATCCCTCGCCAAGGAGCTCCACGCGTGGAGGCGTGAACGCGTGCGGGGACCGCCACAA
CGACAACCGTCGCAGCAGCCCCAGCTCAAGGCCCAGCCGAGGCCGCGCGCAGGAACGCCACCC
AGGGGAGGGGGGGGGGGAGGCGACCTCCTGTTTGGCAGTGGTATCGTGTTCAACTTCGACCTT
GACTGTCCGGAGGAGGAGGATGACCTTGACCGAGCTTTCCCTGGGGGGCTCGTACCTGGCCGC
TCTGTTGGTGTTGGTGGTAGTGGTGGTGGCGATCGAGAAGGTCGAAGCGGGGGTTTCGCTGCG
GAAGGAGGCGTTACGGGCCTAGGGTTTCCGGGGGGGGCTCCTGGCCTGGAGGGGGTAGGGGGT
GGTAGCGCGGCGGAGGTGGCGGAGGGGGGGGTGGAGGCGAGGGCTGTCGATGCCCACGAACCT
TGGAAGGGAGAGGAGAGGGGGGAGGGGGGAGGGGGGGGGGGGTGGAAGGGGGGCGCGGAGGAG
GAGAAGGGGGAGTGGAAAGATGCATCACGCCCCGCTCTGCCGCCTGCCTTGGGTGGAAAGAAG
GGGGCTGTGGCGGCCGAGGATGAGTTTGATGCCGGCTGGCTGTACTCCAGGCAGCCGCGCGAT
AGGAAGAGCGTTGTCGCGGAAGCAAAGCAGTGTAGCAAAGGGACTACAACGCCGCACACGCGG
GGCTTCCCGGAGGCGTACGATGTGCGGGTCGAGGGGGCGCTGCCGTCGGGCTCTCGGGCCTTC
GCTTCCTTTACTCCGCCCTTTGCCTTTACTATTGAAGTGTTGTTGGGGTCCCTGTTCGACCTC
CTCGGGGAGGCGGGCCTGGAGCTGATGATGGACCTCGTAGCCAAGACGGCGTCGCTCAGACGG
ATCGATGCTCGCGACCTGTTTGCCATAGCCGACGCCCATGGTGGTGGTGGTGGTGGTGGCGGC
GCAGGGGGCGTAGGGGGTGGTGCCGGGGGCGCGGCGGGTATGGGGGGTGTGGGAGGGTTCGGG
GGAGCGCCGGAGGAGGCGCAAGGAAGATCGGGGCCGAGCATCTCCCATCAAATCAAGATTTGG
TCCACTTCGGAGAAAGAAGCGGAGAAGGCTCGCCGGAAAAATCTCAAGAAGGCCGCGAAGCGG
GGGCAGAGGGGGCCCGGGGGGGAGGAAGGCGGTTCGGCGGCGGGGGGCGCAGGGTTGGACTGG
CTGCAGTCCGTGGGATTCGAGGACGAATACCTGAAACAGGAGCGCCTGCTGGGTTTGCAAGGA
GGCGGGACGCGAGCGGGAGGAGGGGACAACGAGGAAATGATGCTGGCGTCCCTTGCTCCGGAG
GGGACGCGGGAGTGGCATGACAGGCGCTCGGGGCTGCCTGCGGGAGCGACGAAAACAGTGGTG
GCCGGCCAGTATGAGCAGGTCCACATCCCCCCTCCGAAACTGCCGGGCCGAAAAGACGGGGAT
GACCTGGTGCCCATCACTGAGCTCGAGCAGTGGGCTCAGCTGGCCTTCAAGGGGACCAAGCGG
CTCAACCCTATGCAGAGCAAGGCGAGCCAACTGCGCGGCTCGAGGCTTGCGGCTCGCGTATAC
CATGCCGCGTTCAGAACTTCGGAAAACCTTCTCGTGTGCGCCCCAACAGGAGCGGGAAAGACG
AACGTGGCTATGCTCTCGCTTCTCCAGCTGGTGAGGCAGCACATCCACGGGGGTGTTCTGGAG
CGGAGCGGTATCAAGGCTATCTACGTGGCCCCGATGAAGGCGCTGGCTCAGGAGGTTGTCGCC
AAGTTCTCCCAGAGACTGAAGCCGCTGGGATTGGTGGTACGGGAGTACACCGGTGACATGCAG
CTGTCCAAGCAAGAGGTGGAAGGGTCGCAGGTGATTGTGACGACTCCCGAGAAGTGGGACGTG
GTGACCCGCAAGGGGGGCGACGGTTCGCTCGTTTCCTCCGTCGGGCTGATCATGATCGACGAG
GTGCACCTCCTTGCCGACGATCGCGGTGCTGTCATCGAGAGTATCGTGGCCAGGACGCAGCGG
TACATGGAGACCACGCAGACGCTCATCCGGCTAGTCGGGCTGTCGGCTACGCTGCCCAATTAC
CAGGACGTTGCGTCCTTCCTCAGGGTCAACCCGAGCAAAGGGCTGTTTTACTTCGGGCCGGAG
CACAGACCAGTCCCGCTGGAGCAGACTTTCATCGGGGTTACCGAGAAGCAGAGGGTTCGGCAG
CAGGCGATGATGAACCGAATCGCCTATCAGAGAGCGAGGGAATCTCTTCAGCGCGGTCATCAG
GTCATGGTGTTCGTGCATGCGCGGAAGGATACCGTCAGAACGGCTGAGGCTATTTTGGAGCTA
GCTCAGCGGGACAACGCCTTCGATGAATTTTCCTGCACGGGCTCGGAGCACTGGGGGCGGCAT
GCCCGCCAGGTTGAAAAATCTCGCAACAAGGAGCTGCGGGACTTGTTTCAGGCGGGGCTGGGC
TGCCACCACGCCGGGATGCTGCGGTCCGACCGGGGGCTGACGGAGCGATCGTTCGAAGATGGA
GCCATCAAGGTTCTCGTGTGCACGGCTACTCTGGCGTGGGGCGTCAACTTGCCGGCGCACACG
GTGATCATCAAGGGCACGGAGGTGTACAACCCGGAGAAGGGAGGCTTGCAGGACATCAGCATG
CTCGACGTGCTGCAGGTGTTCGGGCGAGCCGGACGCCCCCAGTACGACACGAGCGGGGAGGCC
ATCATGATCACCACGCACAAGTCGCTTGACAAATACCTGGCCCTGCTGGCCAAGCAAACGCCG
ATCGAGAGCTCCTTCATCAAGGCCTTGCCCGATCATCTCAATGCGGAAGTGTCTTCGGGAACT
GTCACCACTGTCGACGAGGGCGTGACGTGGCTGTCCTACACGTACCTTCATGTCAGAATGCGG
CGGAACCCGATGGCGTACGGCGTGCCTCTGTTGGACCGCGAGGCAGACCCTCAGCTGCTTGAG
AGGCGGAGGCAGCTCATCACCCAGGCGGCAGAAACGCTAGACGACCACAAGATGTTGCGGTTC
GACCGCCGATCGGGTAATCTGGCGGTTACGGACTTGGGACGCGCCGCCTCCCATTTCTACATC
AGCCACGAGAGCGTGTTCCGCTTCAACGGCGCGATGATGCCAACTCTCTCCGATGCCGCGGCC
CTCAACACGGTCTGCCTTGCGAGCGAGTTCGACCAGGTCAAGGTTCGTCCCGAGGAGCTGAAG
GACATGGACAACATGCGGAAACGCTGCCCCCTCGAGGTGAAGGCTCCGCTGGAGGAGTCTGCC
GGGAAGGTCAACGTGCTCCTGCAGTCCTACATCGGCGGGGGGAGGCCCCTCAGCTTCACGCTT
GCGTCGGACACCAACTATGTCGCTCAAAACGCCGGACGCGTGTCCCGTGCGATATTCGAGATC
GCCCTGCGTAAGGGGTGGTGCGGACTTGCGCTCACCATGCTGGAGTTGAGCAAGGCCATCGAC
CGCCGTGTTTGGTGGTTTCAGTCACCGCTCCGACAGTTCGGCGTGCTCCCGGGCACGGTCCTC
ATGAACCTCGAAGGCAAGGGGGGGGGGGGATCGGAAGGGATCGGGAGGCTGCTCGACATGGAT
GCCGGGGAGGTCGGGGCGCTTTGCCACAACCACCGCATGGGGGACACGGTGCTGCGGCTGGCA
AGATCTTTACCGGCTCTGCACATCGAAACGGCCGTGCAGCCGGTTACTCGCGGCATCCTCAGG
CTGACACTCAGGGTTCACGCAAGCTTCAAATGGCAGGACAAGTTCAACGGTTCCACGGAGTCG
TTTTGGATCTGGGTGGAGGACGGGGAAAACGAGCACGTCTATCACTCCGAGACCTTCCTCCTG
AAGAAAAAAAAAAGGAACGAAGCGCAAGAACTCTCCTTCAACATCCCGGTCTTCGAGCCCCTC
CCCGCCCAGTACTGGGTGAGGTGGTGCAGCGACCGCTGGGTGGGCTGCGAGGATGTGCAGCCC
GTGTCCTTCCAGCACCTTGTCTTGCCGGAGAGGTACCCCGCACACACAACCCTCCTGGACCTG
CGTCCTTTGCCCATCACAGCGCTGCAGAACCCCAAGTTTGAGAGCTTGTACCGGTACGAACAC
TTCAACCCTATCCAGACGCAGCTGTTCCATGTGCTCTACCACAGCGACGAAAGCGTCTTGTTG
GGGGCGCCGACAGGGTCCGGGAAGACTGCCGTCGCGGAGATCGCCATCATGCGCATGCTCAAC
GAGCACCCGGGGGCGAAGGCGGTGTACGTGGCACCTCTCAAGGCCCTCGCCCGAGAGCGCCTC
AAAGATTGGCGGGAGAAGTTCGAGAAGAAGATGGGCATGGGCGTTCTGGAGCTCACCGGCGAC
AATACCCCGGACGTAGACGCGCTCAAGCGGTCGACTATCATCGTCACCACCCCCGAGAAGTGG
GATGGCGTTACGAGGAGCTGGAAGACTCGGGACTACGTCAAGGACACCGGCTTAGTGATCATG
GATGAGATCCACTTGCTCGGGGAGGACCGAGGGCCAGTCCTCGAGGTGATCGTCAGTCGGATG
CGGTACATCGCGGCGTCCGCCGCTCGCGGCAACGGCTCCTCCGGCCAACGACAGGTGCGGTTC
GTGGGCTTGAGCACGGCGCTCGCCAACCCGAGGGACCTGTCGGACTGGCTCGGAGTGAAGGAC
ACCGGCCTCTACAACTTCAGGCCTAGCGTCCGGCCCATCCCCTGCGAAGTTCACATCCAGGGA
TACCCCGGCAAGCATTACTGCCCGCGCATGGCGAGCATGAACAAGCCTACGTACGCGGCCATT
TTGGAGCACAGCCCGGAGAAACCGGTGCTGGTGTTCGTCGCTTCTCGGCGGCAGACGCGGCTG
ACAGCCTTGGACCTGATCAGCCTTTGCGCAAGGGCGGACAACCCGAGGCGGTTCGTGCGGATG
CCGGAGGATGAGGCGCAGAACGCCGCGGATTCTGTCAGAGATCAAGCGCTGCAGCACACTCTC
GCGTTCGGAATCGGCATCCACCACGCCGGTTTGGCCGAGGGTGACCGGAGCCTCGTGGAGGCG
TTGTTTGAACAGGGGAAGATTCAGGTACTGGTGTGCACGTCTACCCTAGCGTGGGGCGTGAAC
TTCCCCGCCCACCTCGTCGTGGTGAAGGGAACCGAGTTTTTCGACGGCAAGAGTCAACGATAC
GTCGACTTCCCCATCACGGATCTCTTGCAGATGATTGGGCGCGCCGGACGTCCCCAGTTCGAC
GACCACGCCGTCGCCTGCATCCTCGTCCACGAGCCCAAGAAAAACTTCTTCAAGAAGTTTCTG
TACGAGCCCTTCCCGGTGGAGTCTAAGCTGCCCGGGTCGCTGCACAACCACTTGAGCGCCGAG
TGCGCGGGAGGGGCGATCAAGTCCCGCAAGGATGCGGTGGACTACCTGACGTGGACCTTCTAC
TTTGTGAGGCTGCTGGCGAACCCGTCCTTCTATGGTCTGACGGAGACGTCCACGGATGGCGTT
CAGGAGGTGCGGGCTACCCCGTTGGCCATGGTGGCGAGCCGGTACTACCTGGACTACCGCACC
ATGAAGCTGTTCCAGGGATGCTTCGGCGGGAAAGGAGGGGATGGGGCTACTTTGGAAGACCTC
TGTCGCCTGCTCGCTGATGCCCAGGAGTATGCTGAGCTCCCCGTGCGACACAACGAAGACGTT
CTGAACGGAGAACTTTCCAAGAAGCTTCCGTGGGCTGTGGAAACCGAGGAGCTCGACAGCCCG
CACGCCAAGGCTCACCTTCTGCTGCAGGCCCACTTCGACCGCTGCACTCTGCCGATATCGGAC
TACGTCACAGACACGAGGAGCGTGTTGGATCAGGCCGTGCGCGTGCTAAACGCCATGCTAGAC
ATCGCCGCTGGGTTTGGGTTGCTGGAGACCACCCTGGGGTTGCTGCGGCTGCACCAGATGTTG
GTCCAGGAGTTGGTGCAGGCGGGTGACTGCGAAGCTACCGTGCGCGTTTCGGTGGCCGCGCCG
CACGGCCGGAGAGGCTCGCTCTGGTCTCCGCGATTCCCGAGGGCTCAGAACGTGGGCTGGTGG
CTGGTGCTGGGCACGGAAGAAGGGGAGCTGCTGGCTCTCAAGAGGGGTACAACAACAGCAGCA
GCGGTAGTCCACGAATTAAAAGGCAAACGTTCGACCCCCGTCGTGATACGTAGTCGTGATAGG
CGTGGGGCGCAAACGGTGCCGGCAAAAAAATGCGGCCAGGGTCACAGCACCTCCTGCAACCAT
GGCAGCATCAGAATCGCCGCCAGTCACAACGTCAGGCATGACGAGCGAACACCAACCGCACGG
CGCAAAGGACATAATTTCAACGCACACAACGTCTACCGTGAAAAAAAAATGCCGACATAAGGC
AACGCCCGGGACCACCACGGAGGGACAGCGGCGGAAACGTTAAACTAAAGCATACTGGTAAAA
GTGGTAATAAGTGGTTGATCGTTATGCTACGTGA

A mutation for use in an algal gametophyte or method described herein can be, alternatively or additionally, a mutation in SAMSARA and/or OUROBOROS.

As used herein, “SAMSARA” or “SAM” is a three amino acid loop extension homeodomain transcription factor (TALE HD TF). Sequences for algal SAMSARA are known in the art, e.g., the Macrocystis pyrifera SAMSARA protein, transcript, and genome sequences are available, respectively, under the name CE883644, Protein ID: 883645, Location ID: scaffold 34:3864692-3879075 in the JGI PhycoCosm database. The SAMSARA protein for Ectocarpus is available under GenBank ID ANN44523 or GI 1040348337.

In some embodiments of any of the aspects, SAMSARA is a gene encoding a transcript at least 80% identical to SEQ ID NO: 1. In some embodiments of any of the aspects, SAMSARA is a gene encoding a transcript at least 85% identical to SEQ ID NO: 1. In some embodiments of any of the aspects, SAMSARA is a gene encoding a transcript at least 90% identical to SEQ ID NO: 1. In some embodiments of any of the aspects, SAMSARA is a gene encoding a transcript at least 95% identical to SEQ ID NO: 1. In some embodiments of any of the aspects, SAMSARA is a naturally-occuring gene encoding a transcript at least 95% identical to SEQ ID NO: 1. In some embodiments of any of the aspects, SAMSARA is a gene encoding a transcript with the sequence of SEQ ID NO: 1.

>jgi|SlaSLCT1FG3_1|3385191|estExt_fgenesh1_pg.
C_690044 proteinId=3385191 transcriptId=3386447
(Samsara)
SEQ ID NO: 1
ATGCTCCTCCGAACACGTCTCGTCGCAGAGAGCGACAAGGGGGGGTCTACGATCGAAGTCGAG
ATAGCGACCCTCGAGCGCCTGAATCAGGAGGACACAAGGCTCTGCGACGATCTGCGCGCGTCA
TGCCGCATAGGGAACAACACCCGCGGGGGGGGCGTGCAGCAGGGCAAGCTCTCCTGCTGGGCC
CTGGTCCAAGGCGGTGGGGTTTCCAAAAGGGCGTTTTCGTCGTCGACTGCTAAAGCACTCATC
GATGGGGCCGTTTCCGGTAGACGCCAGAGCACCGGAACAGGGAGCGGCGACGACGACAGCGGG
GGAAGCAATCCAGGGTCTCGGAAACGTGTTTTCTCAAAGTACGATGAGGAGACGACGAGCATC
TTGACGGAATGGTTTCTCTCGCACAAGAGGTGGCCCTACCCAGCTGCCAAGGAGAAGGATGCC
CTGGCTGAGGCTACCAACCTCACCACCTTGCAGATTTCAAACTGGTTCACCAACAAGCGGAAA
AGGCACTGGACTCCGGTCATCAAGAAGCGCACGAGGTCACCCCGGGACTTCTTCGAGTTCTGC
GTCATGAACCCGAAAGACCACGCGCTAAACAACGTGCGGCCGCACAGCCACCAGCCCCCCCAG
CGGGCTGGCGTAGCTGTGGGCGCGGACAGCAACCCTTACACCCTTGCGGCCATGGCGCAGCGG
GGCGGCATCAACGGCCTTCATGTGCCGGTGGTGGGCGACATGCGCCAGATGGCGGGAGCGGTC
CATCCTGGAGCCGCGCGGGGCTTTACGACGCCAGGTCATAGTGGCCTGCCCGGCATAAGCTTC
GACGGCCTGCCCACTGCCCCCGCGGATGCAGCACACAACAGCGTTGTTATGCCGGGGGGGGGG
GGCGGCGGCGGAGGGTGGGTTCGCACAGCCGCCGGGATGGAGAAGAAGGGCGTCGACAACGCC
GTTGCATGGCTGGACCATTCGCGGCAGCAGCAGCAGCAGCAGGTACAGCAGAGTCAGCAGCAG
CAGCCGCAGCAGCAGCAGCAACAAGGCCAGCACCCTGGCTCGCTTGCTCCCGGCCTGATGTAC
GCTTCCAAGGCCGCCCTGCATGCCGCTAACATGGAAAACTCCACCCCCCATCACCCCGTGCCT
CTGTGGCTCGCGCCGCCCACGGGCATGGACCAGCGGAGGCCGCACCCCATGGGTTATCCTGAC
GCCAACCACAGACCCTCTCCTGCCGGAGCTTCTTCCATCGCGATACCTCAGCACGCGGACGAG
CACAGGGATGCGGCGCGAATGAGGCTGCACGCCCCGCAGTACGCCGATGTCCGCGGGTTTGAT
GCAAGCAAATCACAGCAGAGGTTTATCGAACATCCCGCCTCGTTTTACTCCAAAGGACCGGCC
GGTCCCGACGTGATGCACTATCCCGCCGCACCTTCCAACGCCAACACGGGAGGCGCGATTTCC
GGCGGCGGCGGCGGCGACGAGTACGCTTGGCGCGATGCTGCCGTGAGGCCTGAGACTTTTCCT
CCCATTGCTGCGTTCGCCGCTAGCGGGGGCAAAACCACCAGCAACGAAAAGTGTGACCATGTC
ACTGCCTATGCCAGCACCGGGGACCAAAACCCACTGCCAGAGAGAAAGGGATCACGAGGGTAT
GGGCGTCTTGCCAGCTCTGGCGACAGGCTGGAGGCGGGTGGAGAGGGCATCAGCTTGCAGGAG
ACGCAGGCGAGGATCCAGGAACTGGAGAGGAGCGAGTTCGATCTCAAGATGAGGCTGTTTTAC
GCCGAGGAACAGCTTGAGAACGCTGCCGGGCGAGCGGATGCGGTACAACTTCATCGGGAGGTC
GCAGACGCAAAGCGGCGGAGAGCCCACGCCGAGGACCGCACACGAGTGGCGGGCGAGGCCCTT
GCCGGTTTGGAGGCCGACTGCAGGAAGCTTCAAGCTCAGGTGCGAGATGTGGAAGCGAGGAGG
AAGAAGGACCAGGAGACAAGGGCCGAGGTCGAAGACTCAGCCACGTTCAGGCTAAAGAAACAC
ATAATGCTTCTCGAGGATACCCTCCGTGCAGAGCGTGTCGAGAACGCTCGCCTTCGTGGCGCC
AGCACCGGCTGCCGAGGCAGTGACGACGACCTACCCGGGGAGGCTGGACTGAGACGCTCCAGA
ATACGCCGAAGAAGCACTGGAGACGACGGGCTTGGAAACCCGAACCCCTGGAGTTCTGCCCGT
GGCAACGTCGGCGGCTGTTTTGAGCCCAAGCAGCGAGAGCAAGAGAGGCAGCTGGTGGCCAAG
GTCGGGGCGCAAGATTCGTCAATGCGGTTGCTCCGCGCCGACGTAACCACCTTGAGGAAACGC
TTACGGAGACAGGGGGAGGTCCTGGCGAAACAGATAGAGGAGAACGAGGTGGTGCGCCATTCG
GCAGAACAGGTGGCCTGTGTTGAGGCGGAGGAAATCTCGAGGCTAACGGAGGCACTCGAGCAA
GCTGTAGCGGCAGCCAGTCGAGAGACCGCGAAGCGACGGACGGCGGAAAAACGACTGGCGGCG
CTAGATACACGCCTGATAGCGTTGAAAACCAACGGCACACGCACGCCGCGATCGGTCTCCCGG
ACACCGTCCCCCCACTCTAACACGCGAAGCAATGACGGGTCTACGATGGATACAGGATGGGCG
GCTCACAGGAAGCCTAGGTCTCCCGCCATGGGTGCCTCCTCCCCCTCTTCAGCGTGGACGGGT
AGCGGGAGCCGCGAGCCGAACCGCGGGAGGGAAACCAAGTCGCTCCCGCCGCCCAAGAGGGGG
GCCACCGGCAGCACTGCCGCCTCCAGGGGGGGGCGGCTTGCCCGGAACAGCACAGGTGCGCGC
CGGGAGGGGGGTCGCGTTTGCGAAAAAGGACGGTCCTCATCTTCCCGGGGGGGGAGTCGAGAC
TCCGAGAAGGAACATTGGAGGCCGCCAGGATGTTCTCCTCCGCCAAGCCCTGTTGGTCGACGG
GGCGGGCGAAGGGCCAGCTTCTCGGGGGGGGGGGGCGCTGCTCATGGCCGCCACCACCCCCGC
GGCCGTGGACGGGCAGGAGAGGTCGGGCCCGCCCCTGACCTTAAAACCTCCTCGCTTGCTCTT
TCGAACGAATCGGTCAAAACTGGCGCTAGCGGCGGTGGTGGCGGTGGACGAGGGCGAGAAGCT
GTATCTCCTTTTGATACAGGGCGGTTGGGGGCGAGCGAGAGATGTGCCAGCCCGCTGCGAAAC
GGAGAGGGGCCGTCGGATCGCTCCTCCGTAGGGAGGCGCAAGCGTGGGGATTGGAAGGGGGGT
GTTGACGGTGGCTTGGGAGGCTCTTGCCGGCCCAGGGTGAGCGGCGGTGGTAGCTGCGGCGTC
GCAGGCGCAGGCAGGTTATCCCGCAATTCTGTGGCGAGCCGCGCCTCTACTTCCACGGGGGGG
GCGACTGAGGAAGATGTACACAAACATGTTGCTGCTCGTGGTACGAGTTCTGGGCTGCCACCA
GAAAGACAATCAGACGTTGCCCTGAAAGGGCAGGGCAGCGGTGCCGGTGACGCGCGTGTAATA
AGCACGCCTGCCGACGATGTTTGGTCCGGCAGGATGTCGGAGGTGGGTGCCATGGCGGCCGCT
ATCCGCGGCGTGGATTTGTCCGAAACCGTCGCCCGGGAGCTCCGCTTGGCCGTGGCTACCACC
GCCGCCCCCGCTACCGCTGCCGCACTGCCTGGTTACCGCGCTACTCGCGGCGCGAGGGGGGGG
ATCGCGGAGGGCAGGGCGAGGAAAAGCACGCCGGCTTACGGTGCCGGTAACGAAGGCGAGAAG
ACTTCCCCGCGAAGCGATTGCGGGGACGAGCGGCGGAACGGCGCGCCCGAGTCCTGGGAGACT
AGTGAGGCGCGGAGCCGGCACCGGCGCGGCGGAAGCGAGTACACGAGTGGGGGTTTCAAGATG
AATGGCAGAGTCTTAGGGGGGTCGCCGGGGGATGGGGGGCGGGCGCTCAAGAGCAGAGAAGTT
GTGGAACTAGAGAGTGATGTTCAGCACATATTCCAGTTTTTTGAGGCACGGGATCGAGTGGAT
ACAGTTGCCCGGGAACGCTCGGGGTTGGCCGGGGGAAGAGGAGGAGGGGATAGTGAGGAGGGA
GAGGACGAGGATTTTTTCTCCGCCATGGCTGGGGCACTTGCTTGA

As used herein, “OUROBOROS” or “ORO” is a three amino acid loop extension homeodomain transcription factor (TALE HD TF). Sequences for algal OUROBOROS are known in the art, e.g., the Macrocystis pyrifera OUROBOROS protein, transcript, and genome sequences are available, respectively, under Protein ID: 9464632, Transcript ID: 9465670, and Location: scaffold_7: 8251745-8257249 in the JGI PhycoCosm database. The sequence for OUROBOROS in, e.g, giant kelp is fgenesh2_kg.7_#_8680_#_Contig6917.

In some embodiments of any of the aspects, OUROBOROS is a gene encoding a transcript at least 80% identical to SEQ ID NO: 2. In some embodiments of any of the aspects, OUROBOROS is a gene encoding a transcript at least 85% identical to SEQ ID NO: 2. In some embodiments of any of the aspects, OUROBOROS is a gene encoding a transcript at least 90% identical to SEQ ID NO: 2. In some embodiments of any of the aspects, OUROBOROS is a gene encoding a transcript at least 95% identical to SEQ ID NO: 2. In some embodiments of any of the aspects, OUROBOROS is a naturally-occuring gene encoding a transcript at least 95% identical to SEQ ID NO: 2. In some embodiments of any of the aspects, OUROBOROS is a gene encoding a transcript with the sequence of SEQ ID NO: 2.

>jgi|SlaSLCT1FG3_1|2994255|fgenesh1_kg.287_#_
1269_#_TRINITY_DN9288_c1_g1_i6 proteinId =
2994255 transcriptId = 2995511(Ouroboros)
SEQ ID NO: 2
ATGGACGTAGACGTGCCACCGGGATCAAGAGCCGCCTTGGGAGCCCTGCC
CCCGGGGAACACGATGGTGCCGACTGGCGATCGCGGCACTGGTGTTGACA
TCCGACCTGCTGATTcGGCACCGACTCTCGCGAAAGGACTCCTGCCAGCT
GCCCCTCCAGCGGCAACACAAACAGGCGAAATCATGGTGCCTCCCGGCCC
CTCCACTTCGGACTCGATGGCCACCAAGGATCAGCTTGTCCAGGCCCAGG
AGTACCTGGGAACGCTCCGCTCGGACCGCGCGGAATCGTGCCGGGAGCTG
GAGGAGGAGAGGAAGGTTTTTCTGGACTCCCCCTCCACCCCTGAGCAGGA
ACGGCGCTTCCGCCTCATCTACCAGCTTCTGCGATACCAGGTCCACACCC
ACACCCACAGCATGTATTCTATCCTGCAGGAGAAGCTGGCGGCGATGGCG
AGGGTACAGGCTATGGTTGGCGGCGGCGAGGACGACCCGGATGTCAAAGC
CCTCACAATCGCACTAGTCAACGGGAGAATTGTCGACGCAACGATCAGGG
TGGTAGAGCGCGCCCTCGAGGCGATGCGCGAGCTTAACGGCACGGGCGCG
GTGATGAAGGAACCTTGTCCGCCGTCCGCCTGGGAGCAATCGAAGAAGGA
GCAAGAGCCCACTCTGCCCTGCGTGCCCACTGACCCGTCTGGGCAGAAGC
CGGCCAAGCGATCCCGCTTGCCCGCGGCGGCAGTGAAAGAGATGAGGACG
TGGCTTGACCAGCACTGGGACGACCCTGTGCCCACCCACGAGGAAAAGGC
CGGCTTTGCTCAAAGGCACGACATCACAACCAAGCAGGTCGAAAACTGGT
TCATCAACATCCGCATGAGAGAGTGGCGGCCCGCCATGCGCCGGGCCCTG
GATCACGCCGAGGAGACCGGCTCGTATGCGGAATTCTCGAGGCTCCTCCA
GCACACCGCAGACGGCAACGTCTTCAAGAAGTTCCTCGAGGAGCGCGAGA
TGGGCCACGGCTCCGCCTTGGTGCCCCCACCCAGCCTCCAGCGCTTCATC
GGTGCGGAAGGCTTCGCAGCATTCGTGGGAGAAAGGTCCAACAAGCGCAT
GCGCTGA

Genomic sequences for algae are publicly available, e.g, the JGI PhycoCosm database (available on the world wide web at phycocosm.jgi.doe/gov) provides genome, transcript, and protein sequences for algae such as Macrocystis pyrifera, Ectocarpus siliculosus, Saccharina latissima, Undaria pinnatifida, and others. Further details of algae genomic information and sequences is described, e.g, in Ye et al. Nature Communications 6:6986 (2015); Gonzalez et al. Evolution 77:1354-1369 (2023); Diesel et al. BMC Genomics 24:543 (2023), each of which is incorporated by reference herein in its entirety.

The foregoing gene locations and sequences in the Physocosm sugar kelp genome (except for MRE11, which is provided in the giant kelp genome) are also as detailed in the following table:

| | | Gene Length | # of | Protein | Species used |
|---|---|---|---|---|---|
| Gene Name | Gene Location | (AA) | Exons | ID | to blast |
| Samsara | scaffold_69:1006214-1048925 | 1380 | 13 | 3385191 | ectocarpus |
| Ouroboros | scaffold_287:457933-469458 | 369 | 11 | 2994255 | ectocarpus |
| Rad50 | scaffold_435:151246-170472 | 278 | 8 | 3391912 | yeast |
| Spo11 (TOPVIA) | scaffold_916:64639-78783 | 393 | 10 | 2189955 | ectocarpus |
| HFM1 (MER3, RCK) | scaffold_142:164464-255094 | 2385 | 47 | 3387767 | yeast |
| MRE11 (in giant kelp) | contig_45:2218399-2226452 | 216 | 7 | 75886 | ectocarpus |

In some embodiments of any of the aspects, the mutation of Tables 3-7 is mutation 2. In some embodiments of any of the aspects, the mutation of Tables 3-7 is mutation 2 of Table 3. In some embodiments of any of the aspects, the mutation of Tables 3-7 is mutation 2 of Table 5. Mutation 2 is a frame-shift mutation in scaffold 16 present in several non-reproductive kelps. This frame shift occurs early in the gene (starts at 3336806, ends at 3327359), at position 3336742. This is in the first exon and 61 nucleotides downstream of the start codon. The gene in question is designated SlaSLCT1FG3_1 or gene ID 7616. In some embodiments of any of the aspects, the mutation of Tables 3-7 is a mutation of Macrocystis pyrifera gene 7616 or a homolog or ortholog thereof. In some embodiments of any of the aspects, the mutation of Tables 3-7 is a mutation of Macrocystis pyrifera gene 7616 or a gene with at least 80% sequence identity thereto. In some embodiments of any of the aspects, the mutation of Tables 3-7 is a mutation of Macrocystis pyrifera gene 7616 or a gene with at least 90% sequence identity thereto. In some embodiments of any of the aspects, the mutation of Tables 3-7 is a mutation of Macrocystis pyrifera gene 7616 or a gene with at least 95% sequence identity thereto. In some embodiments of any of the aspects, the mutation of Tables 3-7 is a mutation of Macrocystis pyrifera gene 7616 or a naturally-occurring gene with at least 95% sequence identity thereto. The gene is required for meiotic chromosome segregation. Sequences for algal SlaSLCT1FG3_1 are known in the art, e.g., the Macrocystis pyrifera SlaSLCT1FG3_1 protein, transcript, and genome sequences are available, respectively, under Protein ID: 23333918, Transcript ID: 2335174, and Location: scaffold_16: 3327359-3336806 in the JGI PhycoCosm database.

In some embodiments of any of the aspects, the mutation of Tables 3-7 is mutation 39. In some embodiments of any of the aspects, the mutation of Tables 3-7 is mutation 39, e.g., mutation 39 of Table 3. In some embodiments of any of the aspects, the mutation of Tables 3-7 is mutation 39, e.g., mutation 39 of Table 5. Mutation 39 is an early stop codon mutation in the third exon of the gene. In some embodiments of any of the aspects, the mutation of Tables 3-7 is a mutation of Macrocystis pyrifera gene 23147 or a homolog or ortholog thereof. In some embodiments of any of the aspects, the mutation of Tables 3-7 is a mutation of Macrocystis pyrifera gene 23147 or a gene with at least 80% sequence identity thereto. In some embodiments of any of the aspects, the mutation of Tables 3-7 is a mutation of Macrocystis pyrifera gene 23147 or a gene with at least 90% sequence identity thereto. In some embodiments of any of the aspects, the mutation of Tables 3-7 is a mutation of Macrocystis pyrifera gene 23147 or a gene with at least 95% sequence identity thereto. In some embodiments of any of the aspects, the mutation of Tables 3-7 is a mutation of Macrocystis pyrifera gene 23147 or a naturally-occurring gene with at least 95% sequence identity thereto. The gene is required for meiotic chromosome segregation. E.g.:

| | | | | | | | |
|---|---|---|---|---|---|---|---|
| stop_{—} | scaffold_{—} | 74169 | G | C | gene_{—} | 73680 | 75728 |
| gained | 671 | | | | 23147 | | |

In some embodiments of any of the aspects, the mutation of Tables 3-7 is mutation 5. In some embodiments of any of the aspects, the mutation of Tables 3-7 is mutation 5 of Table 5. In some embodiments of any of the aspects, the mutation of Tables 3-7 is mutation 5 of Table 3. Mutation 5 is a point mutation that changes the start codon of the gene to ATT, thereby knocking out the gene. In some embodiments of any of the aspects, the mutation of Tables 3-7 is a mutation of Macrocystis pyrifera gene 22011 or a homolog or ortholog thereof. In some embodiments of any of the aspects, the mutation of Tables 3-7 is a mutation of Macrocystis pyrifera gene 22011 or a gene with at least 80% sequence identity thereto. In some embodiments of any of the aspects, the mutation of Tables 3-7 is a mutation of Macrocystis pyrifera gene 22011 or a gene with at least 90% sequence identity thereto. In some embodiments of any of the aspects, the mutation of Tables 3-7 is a mutation of Macrocystis pyrifera gene 22011 or a gene with at least 95% sequence identity thereto. In some embodiments of any of the aspects, the mutation of Tables 3-7 is a mutation of Macrocystis pyrifera gene 22011 or a naturally-occurring gene with at least 95% sequence identity thereto. The gene is a multicopper oxidase. E.g.,:

| | | | | | | | |
|---|---|---|---|---|---|---|---|
| start_{—} | scaffold_{—} | 278180 | C | A | gene_{—} | 260188 | 278182 |
| lost | 333 | | | | 22011 | | |

A mutation described herein, e.g., a mutation of OUROBOROS and/or SAMSARA can be a loss-of-function mutation.

As used herein “loss-of-function” refers to partial or complete reduction of the expression or activity of an RNA and/or protein encoded by an endogenous DNA sequence in a cell such that the protein can no longer accomplish its function. In some embodiments of any of the aspects, a loss-of-function allele comprises a modification, e.g., as compared to the wild-type sequence. In some embodiments of any of the aspects, a loss-of-function allele comprises an engineered modification. A “modification” in a nucleic acid sequence refers to any detectable change in the genetic material, e.g., a change or alteration relative to a reference sequence, e.g, the wild-type sequence. Modifications can be insertions, deletions, replacements, indels, SNPs, mutations, substitutions, or the like. A modification is usually a change of one or more deoxyribonucleotides, the modification being obtained by, for example, adding, deleting, inverting, or substituting nucleotides.

In some embodiments of any of the aspects, a loss-of-function mutation comprises, consists of, or consists essentially of an excision (e.g., an engineered excision) of at least part of a coding or regulatory sequence. In some embodiments of any of the aspects, a loss-of-function mutation comprises, consists of, or consists essentially of an excision of a gene's promoter (e.g, an engineered excision). In some embodiments of any of the aspects, a loss-of-function mutation comprises, consists of, or consists essentially of an excision (e.g., an engineered excision) of at least 5%, at least 10%, at least 20%, at least 30% or more of a gene's coding sequence. In some embodiments of any of the aspects, a loss-of-function mutation comprises, consists of, or consists essentially of an excision (e.g., an engineered excision) of at least 90%, at least 95%, or 100% of a gene's coding sequence.

In some embodiments of any of the aspects, a loss-of-function mutation comprises, consists of, or consists essentially of a missense or nonsense mutation (e.g., an engineered missense or nonsense mutation) within the first 70% of the coding sequence of a gene. In some embodiments of any of the aspects, a loss-of-function mutation comprises, consists of, or consists essentially of a missense or nonsense mutation (e.g., an engineered missense or nonsense mutation) within the first 50% of the coding sequence of a gene. In some embodiments of any of the aspects, a loss-of-function mutation comprises, consists of, or consists essentially of a missense or nonsense mutation (e.g., an engineered missense or nonsense mutation) within the first 40% of the coding sequence of a gene. In some embodiments of any of the aspects, a loss-of-function mutation comprises, consists of, or consists essentially of a missense or nonsense mutation (e.g., an engineered missense or nonsense mutation) within the first 30% of the coding sequence of a gene. In some embodiments of any of the aspects, a loss-of-function mutation comprises, consists of, or consists essentially of a missense or nonsense mutation (e.g., an engineered missense or nonsense mutation) within the first 20% of the coding sequence of a gene. In some embodiments of any of the aspects, a loss-of-function mutation comprises, consists of, or consists essentially of a missense or nonsense mutation (e.g., an engineered missense or nonsense mutation) within the first 10% of the coding sequence of a gene.

In some embodiments of any of the aspects, a loss-of-function mutation comprises, consists of, or consists essentially of a knock-out modification, e.g, an engineered knock-out modification. As used herein, “knock-out” refers to partial or complete reduction of the expression of a RNA and/or protein encoded by an endogenous DNA sequence such that the RNA and/or protein can no longer accomplish its function. In some embodiments, the “knock-out” can be produced by targeted deletion of the whole or part of a gene. In some embodiments, the deletion may prevent or reduce the expression of the functional RNA and/or protein in a cell in which it is normally expressed.

In some embodiments of any of the aspects, a knock-out mutation comprises a deletion of the whole or part of a gene. In some embodiments of any of the aspects, a knock-out mutation comprises deletion of the entire coding sequence of the relevant gene. In some embodiments of any of the aspects, a knock-out mutation does not comprise any of the coding sequence of the relevant gene. In some embodiments of any of the aspects, a knock-out mutation comprises deletion of a part of the coding sequence of the relevant gene, e.g, 10%, 20%, 30%, 40%, 50%, 60%, 70%, 80%, 90% or more of the coding sequence of the relevant gene. In some embodiments of any of the aspects, a knock-out mutation comprises a nonsense mutation of the relevant gene, e.g, in the first 10%, first 20%, first 30%, first 40%, first 50%, first 60%, or first 70% of the coding sequence of the relevant gene. In some embodiments of any of the aspects, a knock-out mutation comprises a missense mutation of the relevant gene, e.g, in the first 10%, first 20%, first 30%, first 40%, first 50%, first 60%, or first 70% of the coding sequence of the relevant gene. In some embodiments of any of the aspects, a knock-out mutation comprises the introduction of a stop codon in the relevant gene, e.g, in the first 10%, first 20%, first 30%, first 40%, first 50%, first 60%, or first 70% of the coding sequence of the relevant gene. In some embodiments of any of the aspects, a knock-out mutation comprises deletion of the promoter of the relevant gene, e.g, deletion of at least 40%, at least 50%, at least 60%, at least 70%, at least 80%, at least 90% or more of the promoter of the relevant gene.

A missense mutation can comprise, e.g., a frameshift mutation or an alteration in splicing or intron use.

In some embodiments of any of the aspects, a mutation is an exon loss mutation. In some embodiments of any of the aspects, a mutation is a frameshift variant. In some embodiments of any of the aspects, a mutation is an rare amino acid substitution mutation. In some embodiments of any of the aspects, a mutation is a splice acceptor mutation. In some embodiments of any of the aspects, a mutation is a splice donor mutation. In some embodiments of any of the aspects, a mutation is a start codon loss mutation. In some embodiments of any of the aspects, a mutation is a stop codon gained mutation. In some embodiments of any of the aspects, a mutation is an stop codong loss mutation. In some embodiments of any of the aspects, a mutation is an transcript ablation mutation.

In some embodiments of any of the aspects, the at least a first mutation and the at least a second mutation are not the same mutation. In some embodiments of any of the aspects, the at least a first mutation and the at least a second mutation are mutations in the same gene, but are not the same mutation. In some embodiments of any of the aspects, the at least a first mutation and the at least a second mutation are not mutations in the same gene. In some embodiments of any of the aspects, the at least a first mutation and the at least a second mutation are mutations in different genes.

In some embodiments of any of the aspects, the mutation, the at least a first mutation, or the at least a second mutation comprises a mutation in a gene selected from the group consisting of: Spo11, Mre11, Rad50, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof. In some embodiments of any of the aspects, the mutation, the at least a first mutation, or the at least a second mutation comprises a mutation in a gene selected from the group consisting of: Spo11, Mre11, Rad50, HFM1, SAMSARA, and OUROBOROS. In some embodiments of any of the aspects, the mutation, the at least a first mutation, or the at least a second mutation comprises a mutation in a gene selected from the group consisting of: Spo11, Mre11, Rad50, SAMSARA, and OUROBOROS. In some embodiments of any of the aspects, the mutation, the at least a first mutation, or the at least a second mutation comprises a mutation in a gene selected from the group consisting of: Spo11, Mre11, and Rad50.

In some embodiments of any of the aspects, the at least a first mutation comprises a mutation in Spo11 and the at least a second mutation comprises a mutation in a gene selected from the group consisting of: Mre11, Rad50, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof. In some embodiments of any of the aspects, the at least a first mutation comprises a mutation in Spo11 and the at least a second mutation comprises a mutation in a gene selected from the group consisting of: Mre11, Rad50, HFM1, SAMSARA, and OUROBOROS. In some embodiments of any of the aspects, the at least a first mutation comprises a mutation in Spo11 and the at least a second mutation comprises a mutation in a gene selected from the group consisting of: Mre11, Rad50, SAMSARA, and OUROBOROS.

In some embodiments of any of the aspects, the at least a first mutation comprises a mutation in Mre11 and the at least a second mutation comprises a mutation in a gene selected from the group consisting of: Spo11, Rad50, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof. In some embodiments of any of the aspects, the at least a first mutation comprises a mutation in Mre11 and the at least a second mutation comprises a mutation in a gene selected from the group consisting of: Spo11, Rad50, HFM1, SAMSARA, and OUROBOROS. In some embodiments of any of the aspects, the at least a first mutation comprises a mutation in Mre11 and the at least a second mutation comprises a mutation in a gene selected from the group consisting of: Spo11, Rad50, SAMSARA, and OUROBOROS.

In some embodiments of any of the aspects, the at least a first mutation comprises a mutation in Rad50 and the at least a second mutation comprises a mutation in a gene selected from the group consisting of: Spo11, Mre11, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof. In some embodiments of any of the aspects, the at least a first mutation comprises a mutation in Rad50 and the at least a second mutation comprises a mutation in a gene selected from the group consisting of: Spo11, Mre11, HFM1, SAMSARA, and OUROBOROS. In some embodiments of any of the aspects, the at least a first mutation comprises a mutation in Rad50 and the at least a second mutation comprises a mutation in a gene selected from the group consisting of: Spo11, Mre11, SAMSARA, and OUROBOROS.

In some embodiments of any of the aspects, the at least a first mutation comprises a mutation in HFM1 and the at least a second mutation comprises a mutation in a gene selected from the group consisting of: Spo11, Mre11, Rad50, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof. In some embodiments of any of the aspects, the at least a first mutation comprises a mutation in HFM1 and the at least a second mutation comprises a mutation in a gene selected from the group consisting of: Spo11, Mre11, Rad50, SAMSARA, and OUROBOROS.

In some embodiments of any of the aspects, the at least a first mutation comprises a mutation in SAMSARA and the at least a second mutation comprises a mutation in a gene selected from the group consisting of: Spo11, Mre11, Rad50, HFM1, OUROBOROS, SlaSLCT1FG3_1, Macrocystis pyrifera gene 23147 or a homolog or ortholog thereof, and/or Macrocystis pyrifera gene 22011 or a homolog or ortholog thereof. In some embodiments of any of the aspects, the at least a first mutation comprises a mutation in SAMSARA and the at least a second mutation comprises a mutation in a gene selected from the group consisting of: Spo11, Mre11, Rad50, HFM1, and OUROBOROS. In some embodiments of any of the aspects, the at least a first mutation comprises a mutation in SAMSARA and the at least a second mutation comprises a mutation in a gene selected from the group consisting of: Spo11, Mre11, Rad50, and OUROBOROS.

In some embodiments of any of the aspects, the at least a first mutation comprises a mutation in OUROBOROS and the at least a second mutation comprises a mutation in a gene selected from the group consisting of: Spo11, Mre11, Rad50, HFM1, SAMSARA, SlaSLCT1FG3_1, Macrocystis pyrifera gene 23147 or a homolog or ortholog thereof, and/or Macrocystis pyrifera gene 22011 or a homolog or ortholog thereof. In some embodiments of any of the aspects, the at least a first mutation comprises a mutation in OUROBOROS and the at least a second mutation comprises a mutation in a gene selected from the group consisting of: Spo11, Mre11, Rad50, HFM1, and SAMSARA. In some embodiments of any of the aspects, the at least a first mutation comprises a mutation in OUROBOROS and the at least a second mutation comprises a mutation in a gene selected from the group consisting of: Spo11, Mre11, Rad50, and SAMSARA.

In some embodiments of any of the aspects, the at least a first mutation comprises a mutation in SlaSLCT1FG3_1, and the at least a second mutation comprises a mutation in a gene selected from the group consisting of: Macrocystis pyrifera gene 23147 or a homolog or ortholog thereof, and/or Macrocystis pyrifera gene 22011 or a homolog or ortholog thereof.

In some embodiments of any of the aspects, the at least a first mutation comprises a mutation in Macrocystis pyrifera gene 23147 or a homolog or ortholog thereof, and the at least a second mutation comprises a mutation in Macrocystis pyrifera gene 22011 or a homolog or ortholog thereof.

In some embodiments of any of the aspects, the at least a first mutation consists of a mutation in Spo11 and the at least a second mutation consists of a mutation in a gene selected from the group consisting of: Mre11, Rad50, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof. In some embodiments of any of the aspects, the at least a first mutation consists of a mutation in Spo11 and the at least a second mutation consists of a mutation in a gene selected from the group consisting of: Mre11, Rad50, HFM1, SAMSARA, and OUROBOROS. In some embodiments of any of the aspects, the at least a first mutation consists of a mutation in Spo11 and the at least a second mutation consists of a mutation in a gene selected from the group consisting of: Mre11, Rad50, SAMSARA, and OUROBOROS.

In some embodiments of any of the aspects, the at least a first mutation consists of a mutation in Mre11 and the at least a second mutation consists of a mutation in a gene selected from the group consisting of: Spo11, Rad50, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof. In some embodiments of any of the aspects, the at least a first mutation consists of a mutation in Mre11 and the at least a second mutation consists of a mutation in a gene selected from the group consisting of: Spo11, Rad50, HFM1, SAMSARA, and OUROBOROS. In some embodiments of any of the aspects, the at least a first mutation consists of a mutation in Mre11 and the at least a second mutation consists of a mutation in a gene selected from the group consisting of: Spo11, Rad50, SAMSARA, and OUROBOROS.

In some embodiments of any of the aspects, the at least a first mutation consists of a mutation in Rad50 and the at least a second mutation consists of a mutation in a gene selected from the group consisting of: Spo11, Mre11, HFM1, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof. In some embodiments of any of the aspects, the at least a first mutation consists of a mutation in Rad50 and the at least a second mutation consists of a mutation in a gene selected from the group consisting of: Spo11, Mre11, HFM1, SAMSARA, and OUROBOROS. In some embodiments of any of the aspects, the at least a first mutation consists of a mutation in Rad50 and the at least a second mutation consists of a mutation in a gene selected from the group consisting of: Spo11, Mre11, SAMSARA, and OUROBOROS.

In some embodiments of any of the aspects, the at least a first mutation consists of a mutation in HFM1 and the at least a second mutation consists of a mutation in a gene selected from the group consisting of: Spo11, Mre11, Rad50, SAMSARA, OUROBOROS, SlaSLCT1FG3_1, Macrocystis pyrifera gene 23147 or a homolog or ortholog thereof, and/or Macrocystis pyrifera gene 22011 or a homolog or ortholog thereof. In some embodiments of any of the aspects, the at least a first mutation consists of a mutation in HFM1 and the at least a second mutation consists of a mutation in a gene selected from the group consisting of: Spo11, Mre11, Rad50, SAMSARA, and OUROBOROS.

In some embodiments of any of the aspects, the at least a first mutation consists of a mutation in SAMSARA and the at least a second mutation consists of a mutation in a gene selected from the group consisting of: Spo11, Mre11, Rad50,HFM1, OUROBOROS, SlaSLCT1FG3_1, Macrocystis pyrifera gene 23147 or a homolog or ortholog thereof, and/or Macrocystis pyrifera gene 22011 or a homolog or ortholog thereof. In some embodiments of any of the aspects, the at least a first mutation consists of a mutation in SAMSARA and the at least a second mutation consists of a mutation in a gene selected from the group consisting of: Spo11, Mre11, Rad50,HFM1, and OUROBOROS. In some embodiments of any of the aspects, the at least a first mutation consists of a mutation in SAMSARA and the at least a second mutation consists of a mutation in a gene selected from the group consisting of: Spo11, Mre11, Rad50, and OUROBOROS.

In some embodiments of any of the aspects, the at least a first mutation consists of a mutation in OUROBOROS and the at least a second mutation consists of a mutation in a gene selected from the group consisting of: Spo11, Mre11, Rad50, HFM1, SAMSARA, SlaSLCT1FG3_1, Macrocystis pyrifera gene 23147 or a homolog or ortholog thereof, and/or Macrocystis pyrifera gene 22011 or a homolog or ortholog thereof. In some embodiments of any of the aspects, the at least a first mutation consists of a mutation in OUROBOROS and the at least a second mutation consists of a mutation in a gene selected from the group consisting of: Spo11, Mre11, Rad50, HFM1, and SAMSARA. In some embodiments of any of the aspects, the at least a first mutation consists of a mutation in OUROBOROS and the at least a second mutation consists of a mutation in a gene selected from the group consisting of: Spo11, Mre11, Rad50, and SAMSARA.

In some embodiments of any of the aspects, the at least a first mutation consists of a mutation in SlaSLCT1FG3_1, and the at least a second mutation consists of a mutation in a gene selected from the group consisting of: Spo11, Mre11, Rad50, HFM1, SAMSARA, OUROBOROS, Macrocystis pyrifera gene 23147 or a homolog or ortholog thereof, and/or Macrocystis pyrifera gene 22011 or a homolog or ortholog thereof.

In some embodiments of any of the aspects, the at least a first mutation consists of a mutation in Macrocystis pyrifera gene 23147 or a homolog or ortholog thereof, and the at least a second mutation consists of a mutation in a gene selected from the group consisting of: Spo11, Mre11, Rad50, HFM1, SAMSARA, OUROBOROS, SlaSLCT1FG3_1, and/or Macrocystis pyrifera gene 22011 or a homolog or ortholog thereof.

In some embodiments of any of the aspects, the at least a first mutation consists of a mutation in Macrocystis pyrifera gene 22011 or a homolog or ortholog thereof, and the at least a second mutation consists of a mutation in a gene selected from the group consisting of: Spo11, Mre11, Rad50, HFM1, SAMSARA, OUROBOROS, SlaSLCT1FG3_1 and/or Macrocystis pyrifera gene 23147 or a homolog or ortholog thereof.

In some embodiments of any of the aspects, the at least a first mutation comprises a mutation in Spo11 and the at least a second mutation comprises a mutation in Mre11. In some embodiments of any of the aspects, the at least a first mutation comprises a mutation in Spo11 and the at least a second mutation comprises a mutation Rad50. In some embodiments of any of the aspects, the at least a first mutation comprises a mutation in Spo11 and the at least a second mutation comprises a mutation HFM1. In some embodiments of any of the aspects, the at least a first mutation comprises a mutation in Spo11 and the at least a second mutation comprises a mutation SAMSARA. In some embodiments of any of the aspects, the at least a first mutation comprises a mutation in Spo11 and the at least a second mutation comprises a mutation OUROBOROS.

In some embodiments of any of the aspects, the at least a first mutation comprises a mutation in Mre11 and the at least a second mutation comprises a mutation in Rad50. In some embodiments of any of the aspects, the at least a first mutation comprises a mutation in Mre11 and the at least a second mutation comprises a mutation in HFM1. In some embodiments of any of the aspects, the at least a first mutation comprises a mutation in Mre11 and the at least a second mutation comprises a mutation in SAMSARA. In some embodiments of any of the aspects, the at least a first mutation comprises a mutation in Mre11 and the at least a second mutation comprises a mutation in OUROBOROS. In some embodiments of any of the aspects, the at least a first mutation comprises a mutation in Mre11 and the at least a second mutation comprises a mutation in SlaSLCT1FG3_1. In some embodiments of any of the aspects, the at least a first mutation comprises a mutation in Mre11 and the at least a second mutation comprises a mutation in Macrocystis pyrifera gene 23147 or a homolog or ortholog thereof. In some embodiments of any of the aspects, the at least a first mutation comprises a mutation in Mre11 and the at least a second mutation comprises a mutation in Macrocystis pyrifera gene 22011 or a homolog or ortholog thereof.

In some embodiments of any of the aspects, the at least a first mutation comprises a mutation in Rad50 and the at least a second mutation comprises a mutation in HFM1. In some embodiments of any of the aspects, the at least a first mutation comprises a mutation in Rad50 and the at least a second mutation comprises a mutation in SAMSARA. In some embodiments of any of the aspects, the at least a first mutation comprises a mutation in Rad50 and the at least a second mutation comprises a mutation in OUROBOROS. In some embodiments of any of the aspects, the at least a first mutation comprises a mutation in Rad50 and the at least a second mutation comprises a mutation in SlaSLCT1FG3_1. In some embodiments of any of the aspects, the at least a first mutation comprises a mutation in Rad50 and the at least a second mutation comprises a mutation in Macrocystis pyrifera gene 23147 or a homolog or ortholog thereof. In some embodiments of any of the aspects, the at least a first mutation comprises a mutation in Rad50 and the at least a second mutation comprises a mutation in Macrocystis pyrifera gene 22011 or a homolog or ortholog thereof.

In some embodiments of any of the aspects, the at least a first mutation comprises a mutation in HFM1 and the at least a second mutation comprises a mutation in SAMSARA. In some embodiments of any of the aspects, the at least a first mutation comprises a mutation in HFM1 and the at least a second mutation comprises a mutation in OUROBOROS. In some embodiments of any of the aspects, the at least a first mutation comprises a mutation in HFM1 and the at least a second mutation comprises a mutation in SlaSLCT1FG3_1. In some embodiments of any of the aspects, the at least a first mutation comprises a mutation in HFM1 and the at least a second mutation comprises a mutation in Macrocystis pyrifera gene 23147 or a homolog or ortholog thereof. In some embodiments of any of the aspects, the at least a first mutation comprises a mutation in HFM1 and the at least a second mutation comprises a mutation in Macrocystis pyrifera gene 22011 or a homolog or ortholog thereof.

In some embodiments of any of the aspects, the at least a first mutation comprises a mutation in SAMSARA and the at least a second mutation comprises a mutation in OUROBOROS. In some embodiments of any of the aspects, the at least a first mutation comprises a mutation in SAMSARA and the at least a second mutation comprises a mutation in SlaSLCT1FG3_1. In some embodiments of any of the aspects, the at least a first mutation comprises a mutation in SAMSARA and the at least a second mutation comprises a mutation in Macrocystis pyrifera gene 23147 or a homolog or ortholog thereof. In some embodiments of any of the aspects, the at least a first mutation comprises a mutation in SAMSARA and the at least a second mutation comprises a mutation in Macrocystis pyrifera gene 22011 or a homolog or ortholog thereof.

In some embodiments of any of the aspects, the at least a first mutation comprises a first mutation in Spo11 and the at least a second mutation comprises a second mutation in Spo11. In some embodiments of any of the aspects, the at least a first mutation comprises a first mutation in Mre11 and the at least a second mutation comprises a second mutation in Mre11. In some embodiments of any of the aspects, the at least a first mutation comprises a first mutation in RAD50 and the at least a second mutation comprises a second mutation in RAD50. In some embodiments of any of the aspects, the at least a first mutation comprises a first mutation in HFM1 and the at least a second mutation comprises a second mutation in HFM1. In some embodiments of any of the aspects, the at least a first mutation comprises a first mutation in SAMSARA and the at least a second mutation comprises a second mutation in SAMSARA. In some embodiments of any of the aspects, the at least a first mutation comprises a first mutation in OUROBOROS and the at least a second mutation comprises a second mutation in OUROBOROS. In some embodiments of any of the aspects, the at least a first mutation comprises a mutation in SlaSLCT1FG3_1 and the at least a second mutation comprises a mutation in SlaSLCT1FG3_1. In some embodiments of any of the aspects, the at least a first mutation comprises a mutation in Macrocystis pyrifera gene 23147 or a homolog or ortholog thereof and the at least a second mutation comprises a mutation in Macrocystis pyrifera gene 23147 or a homolog or ortholog thereof. In some embodiments of any of the aspects, the at least a first mutation comprises a mutation in Macrocystis pyrifera gene 22011 or a homolog or ortholog thereof and the at least a second mutation comprises a mutation in Macrocystis pyrifera gene 22011 or a homolog or ortholog thereof.

In some embodiments of any of the aspects, the at least a first mutation comprises a first mutation in a first gene described herein and a first mutation in a second gene described herein, and the at least a second mutation comprises a second mutation in the first gene and a second mutation in the second gene, e.g, wherein the first mutation and second mutation in a gene are different mutations. In some embodiments of any of the aspects, the at least a first mutation comprises a first mutation in RAD50 and a first mutation in Spo11, and the at least a second mutation comprises a second mutation in RAD50 and a second mutation in Spo11.

As used herein, “algae” refers to photosynthetic eukaryotes lacking the structures that characterize land plants, e.g, phyllids, sterile cells around their reproductive cells, rhizoids, roots, and leaves. In some embodiments of any of the aspects, the algae are defined as having chlorophyll and lacking phyllids, sterile cells around their reproductive cells, rhizoids, roots, and leaves. In some embodiments of any of the aspects, the algae are multicellular algae, e.g., except for their reproductive cells.

Exemplary algae include green algae, red algae, and brown algae (including kelp).

In some embodiments of any of the aspects, the alga is a brown alga. In some embodiments of any of the aspects, the brown alga is a member of the Stramenopiles clade. In some embodiments of any of the aspects, the brown alga is a member of the Gyrista phylum. In some embodiments of any of the aspects, the brown alga is a member of the Ochrophytina subphylum. In some embodiments of any of the aspects, the brown alga is a member of the Chrysista infraphylum.

In some embodiments of any of the aspects, the alga is a brown alga. In some embodiments of any of the aspects, the brown alga is a member of the Stramenopiles clade. In some embodiments of any of the aspects, the brown alga is a member of the Gyrista phylum. In some embodiments of any of the aspects, the brown alga is a member of the Ochrophytina subphylum. In some embodiments of any of the aspects, the brown alga is a member of the Chrysista infraphylum. In some embodiments of any of the aspects, the brown alga is a member of the Phaeophyceae class.

In some embodiments of any of the aspects, the brown alga is a member of the order Laminariales. In some embodiments of any of the aspects, the Laminariales includes the sugar kelp, Saccharina latissima. In some embodiments of any of the aspects, the alga is Saccharina latissima. In some embodiments of any of the aspects, the Laminariales include a unique subspecies called Saccharina latissima forma angustissima or Saccharina angustissima. In some embodiments of any of the aspects, the alga is Saccharina latissima forma angustissima. In some embodiments of any of the aspects, the alga is Saccharina angustissima. In some embodiments of any of the aspects, the brown alga is the giant kelp. In some embodiments of any of the aspects, the brown alga is the giant kelp, Macrocystis pyrifera.

In some embodiments of any of the aspects, the alga is a green alga (e.g., a member of the Chlorophyta). In some embodiments of any of the aspects, the green alga is an Ulva spp. In some embodiments of any of the aspects, the green alga is a Caulerpa spp. In some embodiments of any of the aspects, the green alga is a Monostroma spp. In some embodiments of any of the aspects, the green alga is a Cladophora spp.

In one aspect of any of the embodiments, described herein is a population of infertile or sterile algal sporophytes. The algal sporophytes can comprise an algal sporophyte(s) as described herein, e.g., comprising mutation(s) as described herein. In some embodiments of any of the aspects, the population comprises at least 100 sporophytes. In some embodiments of any of the aspects, the population comprises at least 1,000 sporophytes. In some embodiments of any of the aspects, the population comprises at least 10,000 sporophytes. In some embodiments of any of the aspects, the population comprises at least 100,000 sporophytes. In some embodiments of any of the aspects, the population consists of infertile or sterile algal sporophytes. In some embodiments of any of the aspects, the population does not comprise fertile algal sporophytes. Such populations of infertile or sterile algal sporophytes cannot occur in nature, as they are incapable of reproduction and would not be able to outcompete fertile algal sporophytes to achieve such numbers and/or concentrations in a population of sporophytes.

In some embodiments of any of the aspects, described herein is a composition of algal gametophytes and/or algal sporophytes as described herein. In some embodiments of any of the aspects, described herein is a combination of algal gametophytes and/or algal sporophytes as described herein.

As used herein, “combination” refers to a group of two or more substances (e.g., cells, organisms, sporophytes, or gametophytes as described herein) for use together, e.g., for cultivation or propagation of the algae. The two or more substances can be present in the same formulation in any molecular or physical arrangement, e.g, in an admixture, in a solution, in a mixture, in a suspension, in a colloid, in an emulsion. The formulation can be a homogeneous or heterogenous mixture. Alternatively, the two or more substances can be present in two or more separate formulations, e.g., in a kit or package comprising multiple formulations in separate containers, to be mixed or brought into contact with each other when, e.g, fertilization is to be performed.

A kit is an assemblage of materials or components, including at least one substance (e.g., cells, organisms, sporophytes, or gametophytes as described herein) described herein. The exact nature of the components configured in the kit depends on its intended purpose. In some embodiments of any of the aspects, a kit includes instructions for use. “Instructions for use” typically include a tangible expression describing the technique to be employed in using the components of the kit, e.g., to perform fertilization to provide a sterile or infertile sporophyte. Still in accordance with the present invention, “instructions for use” may include a tangible expression describing the preparation of at least one element described herein, such as dilution, mixing, or dosing instructions, and the like, typically for an intended purpose. Optionally, the kit also contains other useful components, such as, measuring tools, diluents, buffers, syringes, pharmaceutically acceptable carriers, or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a contaminant-free environment. As used herein, the term “package” refers to a suitable solid matrix or material such as glass, plastic, paper, foil, polyester (such as polyethylene terephthalate, or Mylar) and the like, capable of holding the individual kit components. Thus, for example, a package can be a glass vial used to contain suitable quantities of a composition containing a volume of at least one substance described herein. The packaging material generally has an external label which indicates the contents and/or purpose of the kit and/or its components.

In some aspects of any of the embodiments, provided herein is a method of cultivating or producing algae or algal material, the method comprising contacting an isolated male gametophyte comprising at least one mutation as described herein, with an isolated female gametophyte with at least one mutation as described herein and cultivating or growing the resulting sporophyte. In some aspects of any of the embodiments, provided herein is a method of cultivating or producing algae or algal material, the method comprising cultivating or growing a sporophyte resulting from an isolated male gametophyte comprising at least one mutation as described herein with an isolated female gametophyte comprising at least one mutation as described herein. In some aspects of any of the embodiments, provided herein is a method of cultivating or producing algae or algal material, the method comprising cultivating or growing a population of sporophytes resulting from a population of male gametophytes consisting of one or more isolated male gametophytes comprising at least one mutation as described herein with a population of female gametophytes consisting of one or more isolated female gametophytes comprising at least one mutation as described herein.

In some aspects of any of the embodiments, provided herein is a method of cultivating or producing algae or algal material, the method comprising isolating a male gametophyte comprising at least one mutation as described herein, isolating a female gametophyte with at least one mutation as described herein, contacting the isolated male gametophyte and isolated female gametophyte, and cultivating or growing the resulting sporophyte. In some aspects of any of the embodiments, provided herein is a method of cultivating or producing algae or algal material, the method comprising isolating a male gametophyte comprising at least one mutation as described herein, isolating a female gametophyte with at least one mutation as described herein, and contacting the isolated male gametophyte and isolated female gametophyte. In some aspects of any of the embodiments, provided herein is a method of cultivating or producing algae or algal material, the method comprising isolating a clonal population of male gametophytes comprising at least one mutation as described herein, isolating a clonal population of female gametophytes with at least one mutation as described herein, and contacting the isolated clonal population of male gametophytes with the isolated clonal population of female gametophytes.

In some aspects of any of the embodiments, provided herein is a method of producing, cultivating, or producing sterile or infertile algal sporophytes, the method comprising contacting an isolated male gametophyte comprising at least one mutation as described herein, with an isolated female gametophyte with at least one mutation as described herein. In some aspects of any of the embodiments, provided herein is a method of producing, cultivating, or producing sterile or infertile algal sporophytes, the method comprising contacting an isolated male gametophyte comprising at least one mutation as described herein, with an isolated female gametophyte with at least one mutation as described herein and cultivating or growing the resulting sporophyte. In some aspects of any of the embodiments, provided herein is a method of producing, cultivating, or producing sterile or infertile algal sporophytes, method comprising contacting an isolated clonal population of male gametophytes with an isolated clonal population of female gametophytes and cultivating or growing the resulting sporophyte.

The instant methods are not limited by whether the male or female gametophyte comprises a “first” mutation, e.g., in some embodiments of any of the aspects, the male gametophyte can comprise the at least a first mutation and the female gametophyte can comprise the at least a second mutation and in other embodiments of any of the aspects, the male gametophyte can comprise the at least a second mutation and the female gametophyte can comprise the at least a first mutation.

In some embodiments of any of the aspects, cultivating or growing algae and/or algal material can comprise maintaining the sporophytes(s) in suitable conditions until an adult sporophyte forms. In some embodiments of any of the aspects, cultivating or growing algae and/or algal material can comprise maintaining the sporophytes(s) in suitable conditions until the time or growth stage in which spores (e.g., meiospores) would be released in a wild-type sporophyte has been reached.

In some embodiments of any of the aspects, suitable conditions can comprise maintaining the sporophytes(s) in sea water. In some embodiments of any of the aspects, suitable conditions can comprise maintaining the sporophytes(s) in open sea water.

In some embodiments of any of the aspects, cultivating or growing algae and/or algal material can comprise harvesting the algae or algal material. In some embodiments of any of the aspects, cultivating or growing algae and/or algal material can comprise harvesting the algae or algal material after an adult sporophyte has formed. In some embodiments of any of the aspects, cultivating or growing algae and/or algal material can comprise harvesting the algae or algal material after the sporophyte has reached a size or development stage such that the sporophyte is suitable for use as a commodity or cash crop, or a crop for consumption, or a crop for industrial use.

As used herein, “isolated”, as used with respect to gametophytes or sporophytes means that the gametophyte(s) or sporophyte(s) is not in the presence of a wild-type gametophyte and/or sporophyte. In some embodiments of any of the aspects, an isolated gametophyte is not in contact with, or not present in a composition with, a wild-type gametophyte. In some embodiments of any of the aspects, an isolated gametophyte is in contact with, or present in a composition with, only syngenic gametophytes, e.g, a clonal population of gametophytes. In some embodiments of any of the aspects, an isolated gametophyte is not in contact with, or present in a composition with, gametophytes of the opposite gender.

In one aspect of any of the embodiments, described herein is a biofuel made by maintaining the sporophytes(s) described herein in suitable conditions until an adult sporophyte forms and then producing a biofuel. In some aspects of any of the embodiments, provided herein is a method of producing biofuel, the method comprising cultivating or growing a sporophyte(s) as described herein in suitable conditions; and then producing a biofuel. In some embodiments of any of the aspects, method or process comprises maintaining the sporophytes(s) in suitable conditions until the time or growth stage in which spores (e.g., meiospores) would be released in a wild-type sporophyte has been reached. In some embodiments of any of the aspects, the process or method comprises a first step of contacting an isolated male gametophyte comprising at least one mutation as described herein, with an isolated female gametophyte with at least one mutation as described herein to obtain the sporophyte.

In one aspect of any of the embodiments, described herein is an animal feed, phycocolloid, plant biostimulant, food, cosmeceutical, or nutraceutical made by maintaining the sporophytes(s) described herein in suitable conditions until an adult sporophyte forms and then producing a biofuel. In some aspects of any of the embodiments, provided herein is a method of producing an animal feed, phycocolloid, plant biostimulant, food, cosmeceutical, or nutraceutical, the method comprising cultivating or growing a sporophyte(s) as described herein in suitable conditions; and then producing an animal feed, phycocolloid, plant biostimulant, food, cosmeceutical, or nutraceutical. In some embodiments of any of the aspects, method or process comprises maintaining the sporophytes(s) in suitable conditions until the time or growth stage in which spores (e.g., meiospores) would be released in a wild-type sporophyte has been reached. In some embodiments of any of the aspects, the process or method comprises a first step of contacting an isolated male gametophyte comprising at least one mutation as described herein, with an isolated female gametophyte with at least one mutation as described herein to obtain the sporophyte.

In some aspects of any of the embodiments, provided herein is a method of producing alginate, the method comprising cultivating or growing a sporophyte(s) as described herein in suitable conditions; and then isolating alginate from the sporophyte(s). In some aspects of any of the embodiments, provided herein is a method of producing an algal pigment, the method comprising cultivating or growing a sporophyte(s) as described herein in suitable conditions; and then isolating the algal pigment from the sporophyte(s). In some aspects of any of the embodiments, provided herein is a method of producing fucoxanthin, the method comprising cultivating or growing a sporophyte(s) as described herein in suitable conditions; and then isolating the omega-3 fatty acids from the sporophyte(s). In some aspects of any of the embodiments, provided herein is a method of producing omega-3 fatty acids, the method comprising cultivating or growing a sporophyte(s) as described herein in suitable conditions; and then isolating the omega-3 fatty acids from the sporophyte(s). In some embodiments of any of the aspects, method or process comprises maintaining the sporophytes(s) in suitable conditions until the time or growth stage in which spores (e.g., meiospores) would be released in a wild-type sporophyte has been reached. In some embodiments of any of the aspects, the process or method comprises a first step of contacting an isolated male gametophyte comprising at least one mutation as described herein, with an isolated female gametophyte with at least one mutation as described herein to obtain the sporophyte.

In some embodiments of any of the aspects, the sporophytes or gametophytes present in a composition, or combination, of the disclosure exhibit an increased utility that is not exhibited when said sporophytes or gametophytes occur alone or when said sporophytes or gametophytes are present at a naturally occurring concentration. In some embodiments of any of the aspects, the compositions of the disclosure—comprising isolated gametophytes and combinations thereof as taught herein—exhibit markedly different characteristics/properties compared to their closest naturally occurring counterpart, e.g, they can produce a population of sterile or infertile sporophytes, e.g, not comprising fertile sporophytes. In some embodiments of any of the aspects, the compositions of the disclosure—comprising sporophytes and populations of sporophytes as taught herein—exhibit markedly different characteristics/properties compared to their closest naturally occurring counterpart, e.g, they can be sterile or infertile sporophytes, e.g, not comprising fertile sporophytes. That is, the compositions of the disclosure exhibit markedly different functional and/or structural characteristics/properties, as compared to their closest naturally occurring counterpart. Further, the combinations of mutations described herein are not naturally occurring mutations. Each mutation described herein is exceedingly rare and the likelihood that a gametophyte comprising such a mutation would encounter a gametophyte comprising a different mutation described herein, in nature, is overwhelming statistically unlikely and certain has not been demonstrated to occur. Further, the gametophytes and sporophytes of the disclosure are functionally different from gametophytes and sporophytes as they naturally exists in the ocean, for at least the following reasons: said gametophytes and sporophytes, when utilized in an isolated form can lead to populations or growth of sterile or infertile sporophytes, such that the algae (e.g. the brown algae such as kelp) now have a new utility as a being able to be cultivated without escape events, where the gametophytes and/or sporophytes could not have such a utility in their natural states in the wild, as the appropriate gametophytes would not come in contact with each other without the intervention of the hand of man to formulate the combinations described herein and impart this new utility that has the aforementioned functional characteristics not possessed by the alage in it's natural state of existence in the wild.

The described features, advantages, and characteristics may be combined in any suitable manner in one or more embodiments. One skilled in the relevant art will recognize that the circuit may be practiced without one or more of the specific features or advantages of a particular embodiment. In other instances, additional features and advantages may be recognized in certain embodiments that may not be present in all embodiments.

Reference throughout this specification to “one embodiment,” “an embodiment,” or similar language means that a particular feature, structure, or characteristic described in connection with the embodiment is included in at least one embodiment. Thus, appearances of the phrase “in one embodiment,” “in an embodiment,” and similar language throughout this specification may, but do not necessarily, all refer to the same embodiment.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given mutation or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or level, a “increase” is a statistically significant increase in such level.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification, and processing. Expression can refer to the transcription and stable accumulation of sense (mRNA) or antisense RNA derived from a nucleic acid fragment or fragments of the invention and/or to the translation of mRNA into a polypeptide.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific, and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:
1. An algal gametophyte comprising one or more mutations of Spo11, Mre11, Rad50, HFM1, SAMSARA, OUROBOROS, and Tables 3-7.
2. A combination of algal gametophytes comprising:
   a first algal gametophyte comprising at least a first mutation selected from:
      a mutation of Spo11, Mre11, Rad50, HFM1, SAMSARA, OUROBOROS, and Tables 3-7; and
   a second algal gametophyte comprising at least a second mutation selected from:
      a mutation of Spo11, Mre11, Rad50, HFM1, SAMSARA, OUROBOROS, and/or Tables 3-7.
3. An algal sporophyte comprising:
   at least a first mutation selected from:
      a mutation of Spo11, Mre11, Rad50, HFM1, SAMSARA, OUROBOROS, and/or Tables 3-7; and
   at least a second mutation selected from:
      a mutation of Spo11, Mre11, Rad50, HFM1, SAMSARA, OUROBOROS, and/or Tables 3-7.
4. The algal sporophyte of paragraph 3, wherein the algal sporophyte is infertile or sterile.
5. The algal gametophyte or algal sporophyte of any one of the preceding paragraphs, wherein the at least a first mutation and the at least a second mutation are not the same mutation.
6. The algal gametophyte or algal sporophyte of any one of the preceding paragraphs, wherein the at least a first mutation and the at least a second mutation are not in the same gene.
7. The algal gametophyte or algal sporophyte of any one of the preceding paragraphs, wherein the mutation, the at least a first mutation, or the at least a second mutation comprises a mutation in a gene selected from the group consisting of: Spo11, Mre11, Rad50, HFM1, SAMSARA, and OUROBOROS.
8. The algal gametophyte or algal sporophyte of any one of the preceding paragraphs, wherein the mutation, the at least a first mutation, or the at least a second mutation comprises a mutation in a gene selected from the group consisting of: Spo11, Mre11, HFM1, and Rad50.
9. The algal gametophyte or algal sporophyte of any one of the preceding paragraphs, wherein the at least a first mutation and the at least a second mutation each comprise a mutation in a gene selected from the group consisting of: Spo11, Mre11, Rad50, HFM1, SAMSARA, and OUROBOROS.
10. The algal gametophyte or algal sporophyte of any one of the preceding paragraphs, wherein the at least a first mutation and the at least a second mutation each comprise a mutation in a gene selected from the group consisting of: Spo11, Mre11, HFM1, and Rad50.
11. The algal gametophyte or algal sporophyte of any one of the preceding paragraphs, wherein the at least a first mutation and the at least a second mutation collectively comprise a mutation in each of: Rad50 and Spo11.
12. The algal gametophyte or algal sporophyte of any one of the preceding paragraphs, wherein the at least a first mutation and the at least a second mutation each comprise a different mutation in Rad50.
13. The algal gametophyte or algal sporophyte of any one of the preceding paragraphs, wherein the mutation, the at least a first mutation, or the at least a second mutation comprises a loss-of-function mutation.
14. The algal gametophyte or algal sporophyte of any one of the preceding paragraphs, wherein the mutation, the at least a first mutation, or the at least a second mutation comprises a nonsense mutation.
15. The algal gametophyte or algal sporophyte of any one of the preceding paragraphs, wherein the mutation, the at least a first mutation, or the at least a second mutation comprises a missense mutation.
16. The algal gametophyte or algal sporophyte of any one of the preceding paragraphs, wherein the at least a first mutation and the at least a second mutation are a combination of mutations shown in Tables 3-7.
17. The algal gametophyte or algal sporophyte of any one of the preceding paragraphs, wherein the at least a first mutation and the at least a second mutation are a combination of two mutations shown in Tables 3-7.
18. The algal gametophyte or algal sporophyte of any one of the preceding paragraphs, wherein the at least a first mutation and the at least a second mutation are selected from mutations marked as yes in column SL.OI.11.MG.2, SL-CC-1-GF-2, SA.CB.4.FG.3, or SL-JS-19-MG-1.
19. The algal gametophyte or algal sporophyte of any one of the preceding paragraphs, wherein the at least a first mutation and the at least a second mutation are, collectively, mutations 2 and 9 of Tables 3-7.
20. The algal gametophyte or algal sporophyte of any one of the preceding paragraphs, wherein the algal gametophyte or algal sporophyte is a brown alga (Phaeophyceae class).
21. The algal gametophyte or algal sporophyte of any one of the preceding paragraphs, wherein the algal gametophyte or algal sporophyte is a Laminariales.
22. The algal gametophyte or algal sporophyte of any one of the preceding paragraphs, wherein the algal gametophyte or algal sporophyte is sugar kelp (Saccharina latissima).
23. The algal gametophyte or algal sporophyte of any one of the preceding paragraphs, wherein the algal gametophyte or algal sporophyte is Saccharina latissima forma angustissima.
24. The algal gametophyte or algal sporophyte of any one of the preceding paragraphs, wherein the algal gametophyte or algal sporophyte is giant kelp (Macrocystis pyrifera).
25. The algal gametophyte of any one of the preceding paragraphs, wherein the algal gametophyte is a male gametophyte.
26. The algal gametophyte of any one of the preceding paragraphs, wherein the algal gametophyte is a female gametophyte.
27. A population of infertile, sterile, or non-reproductive algal sporophytes.
28. The population of paragraph 27, wherein the infertile, sterile, or non-reproductive algal spororphytes are the algal sporophytes of any one of the preceding paragraphs.
29. The population of any of paragraphs 27-28, wherein the population does not comprise fertile algal sporophytes.
30. A method of cultivating or producing algae or algal material, comprising:
   a) contacting an isolated male gametophyte of any of paragraphs 1-16 with an isolated female gametophyte of any of paragraphs 1-26; and
   b) cultivating or growing the the resulting sporophyte.
31. A method of cultivating or producing algae or algal material, comprising:
   a) cultivating or growing a sporophyte resulting from an isolated male gametophyte of any of paragraphs 1-26 with an isolated female gametophyte of any of paragraphs 1-26.
32. A method of providing, cultivating, or producing a sterile, infertile, or non-reproductive algal sporophyte, comprising:
   a) contacting an isolated male gametophyte of any of paragraphs 1-26 with an isolated female gametophyte of any of paragraphs 1-26.
33. A method of cultivating or producing alge or algal material, comprising:
   a) cultivating or growing a population of sporophyte resulting from a population of male gametophytes consisting of one or mroe isolated male gametophytes of any of paragraphs 1-26 with a population of female gametophytes consisting of one or more isolated female gametophytes of any of paragraphs 1-26.
34. A composition of algal gametophytes comprising one or more mutations.
35. The composition of paragraph 34, wherein the mutations are selected from genes Spo11, Mre11, HFM1, and Rad50.
36. The composition of any one of paragraphs 34-35, wherein the resulting sporophyte of the gametophytes results in an algal strain unable to reproduce.
37. The composition of any one of paragraphs 34-36, wherein the algal strain is a brown alga.
38. The composition of any one of paragraphs 34-37, wherein the algal strain is Saccharina latissima and/or Saccharina latissima forma angustissima.
39. A plant that is unable to reproduce, wherein the plant comprises one or more mutations or mutated gene products that result in infertility.
40. The plant of paragraph 39, wherein the plant is an alga, e.g., brown alga.
41. The plant of paragraph 40, wherein the alga is Saccharina latissima and/or Saccharina latissima forma angustissima.
42. The plant of any one of paragraphs 39-41, wherein the mutations are selected from genes Spo11, Mre11, HFM1, and Rad50.

The technology described herein is further illustrated by the following examples which in no way should be construed as being further limiting.

### EXAMPLES

TABLE 1
List of Exemplary Strains.

SA18-CB-S2-FG3
SA18-CB-S2-MG3
SA18-CB-S4-FG6
SA18-CB-S5-FG4
SA18-CB-S7-MG2
SA18-CB-S7-MG3
SA18-CB-S9-FG1
SL18-CB-S4-FG6
SL18-CC-S11-FG3
SL18-CC-S12-FG2
SL18-CC-S12-FG3
SL18-CC-S1-FG1
SL18-CC-S1-FG2
SL18-CC-S1-MG3
SL18-CC-S3-FG1
SL18-CC-S3-FG2
SL18-CC-S3-FG3
SL18-CC-S4-MG11
SL18-CC-S5-FG1
SL18-CC-S6-FG3
SL18-CC-S7-MG1
SL18-CC-S8-FG2
SL18-CC-S9-FG1
SL18-FI-S1-FG11
SL18-JS-S11-MG3
SL18-JS-S19-MG1
SL18-JS-S5-MG2
SL18-JS-S6-MG2
SL18-JS-S6-MG3
SL18-JS-S9-FG2
SL18-JS-S9-MG2
SL18-LD-S2-MG2
SL18-LD-S9-FG4
SL18-LD-S9-MG1
SL18-LL-S9-MG4
SL18-NC-S15-MG3
SL18-NC-S2-MG3
SL18-NC-S9-MG2
SL18-NL-S3-FG3
SL18-NL-S7-MG1
SL18-OD-S6-MG1
SL18-OD-S6-MG2
SL18-OD-S6-MG3
SL18-OI-S11-MG2
SL18-OI-S14-MG1
SL18-OI-S15-FG2
SL18-OI-S15-MG2
SL18-OI-S17-FG4
SL18-OI-S18-MG1
SL18-OI-S4-FG2
SL18-OI-S6-FG2
SL18-PI-S1-MG2
SL18-SF-S13-FG2
SL18-SF-S17-FG2

TABLE
Strain Crosses.
Crossed Gametophytes

| Female Gametophyte | Female Mutations | Male Gametophyte | Male Mutations |
|---|---|---|---|
| SL-SF-17-FG-2 | 13 | SA-CB-7-MG-3 | 4, 7, 8, 12, 20, 21, 47 |
| SL-CC-5-FG-3 | 24, 27 | SL-OI-14-MG-1 | 3, 4, 6, 7, 12, 21, 28, 47 |
| SL-OI-4-FG-2 | not_genotyped | SA-CB-7-MG-2 | none |
| SL-OI-17-FG-4 | 13, 24 | SL-JS-6-MG-2 | 9, 13, 43 |
| SL-SF-13-FG-2 | 9 | SA-CB-2-MG-3 | 8, 20, 28 |
| SL-CC-3-FG-1 | 6, 9, 12, 28 | SL-JS-5-MG-2 | 1, 4, 9, 21, 43 |
| SL-CC-12-FG-2 | 9, 47 | SL-LL-9-MG-4 | 8, 13, 19, 21, 23, 38, 40 |
| SA-CB-4-FG-6 | 8, 13 | SL-JS-6-MG-3 | 13, 43 |
| SL-CC-11-FG-3 | not_genotyped | SL-NC-2-MG-3 | not_genotyped |
| SL-OI-6-FG-2 | 4, 13, 17, 24, 28, 40, 47 | SL-LD-2-MG-2 | 3, 4, 7, 8, 13, 21, 23, 31, |
| | | | 40 |
| SL-OI-17-FG-4 | 13, 24 | SL-CC-7-MG-1 | 3, 4, 8, 9, 13, 22, 27, 28, |
| | | | 47 |
| SL-CC-3-FG-2 | 3, 4, 9, 12, 13, 43 | SL-OD-6-MG-1 | 3, 13, 21 |
| SL-SF-13-FG-2 | 9 | SL-JS-11-MG-3 | 3, 5, 21 |
| SA-CB-9-FG-1 | not_genotyped | SL-JS-19-MG-1 | 1, 3, 4, 8, 9, 10, 26, 11, |
| | | | 13 |
| SL-CC-1-FG-1 | 8, 9, 12, 27, 30, 32, 41, 42 | SL-NC-15-MG-3 | not_genotyped |
| SL-NL-3-FG-3 | 8, 13 | SL-NC-9-MG-2 | 1, 4, 13 |
| SL-SF-17-FG-2 | 13 | SA-CB-7-MG-2 | none |
| SL-CC-3-FG-3 | 3, 9, 38, 43 | SL-OD-6-MG-3 | 3, 13, 21, 40, 42 |
| SL-OI-15-FG-2 | not_genotyped | SA-CB-2-MG-3 | 8, 20, 28 |
| SL-SF-13-FG-2 | 9 | SL-JS-9-MG-2 | 7, 8, 9, 43 |
| SA-CB-5-FG-4 | not_genotyped | SL-CC-1-MG-3 | 4, 8, 9, 30, 32, 43 |
| SL-LD-9-FG-4 | not_genotyped | SL-OI-6-MG-2 | 8, 12, 13, 21, 24 |
| SL-CC-8-FG-2 | not_genotyped | SL-LD-9-MG-1 | 3, 4, 8, 9, 12, 13 |
| SL-CC-1-FG-1 | 8, 9, 12, 27, 30, 32, 41, 42 | SL-CC-4-MG-11 | 3, 4, 6, 7, 12, 13, 28, 38, |
| | | | 47 |
| SL18-CC-9-FG1 | not_genotyped | SL18-OI-18-MG1 | not_genotyped |
| SA18-CB-2-FG3 | not_genotyped | SL18-JS-19-MG1 | not_genotyped |
| SL18-CC-12-FG3 | not_genotyped | SL18-OI-15-MG2 | not_genotyped |
| SL18-CC-5-FG1 | not_genotyped | SL18-OI-15-MG2 | not_genotyped |
| SL18-CC-1-FG2 | not_genotyped | SL18-OI-11-MG2 | not_genotyped |
| SL18-CC-6-FG3 | not_genotyped | SL18-CC-7-MG1 | not_genotyped |
| SA18-CB-4-FG6 | not_genotyped | SA18-CB-1-MG13 | not_genotyped |
| SA18-CB-4-FG6 | not_genotyped | SL18-LD-2-MG2 | not_genotyped |
| SA18-CB-7-FG1 | not_genotyped | SL18-UCONN-4-MG3 | not_genotyped |
| SL18-CC-1-FG1 | not_genotyped | SL19-DB-9-MG3 | not_genotyped |
| SL18-CC-1-FG2 | not_genotyped | SL18-OI-11-MG2 | not_genotyped |
| SL18-CC-12-FG2 | not_genotyped | SA18-CB-7-MG1 | not_genotyped |
| SL18-CC-5-FG3 | not_genotyped | SA18-CB-4-MG2 | not_genotyped |
| SL18-CC-S5-FG3 | not_genotyped | SL18-CC-7-MG1 | not_genotyped |
| SL18-CC-5-FG3 | not_genotyped | SL18-O1-6-MG2 | not_genotyped |
| SL18-FI-1-FG6 | not_genotyped | SA18-CB-10-MG1 | not_genotyped |
| SL18-JS-5-FG2 | not_genotyped | SL18-CC-2-MG6 | not_genotyped |
| SL18-LD-13-FG1 | not_genotyped | SL18-LD-2-MG2 | not_genotyped |
| SL18-SF-13-FG2 | not_genotyped | SL18-LL-9-MG4 | not_genotyped |
| SL18-SF-13-FG2 | not_genotyped | SL18-NC-2-MG3 | not_genotyped |
| SL18-UCONN-142-FG2 | not_genotyped | SL18-UCONN-143-MG1 | not_genotyped |
| SL18-UCONN-142-FG2 | not_genotyped | SL19-UCONN-142-MG3 | not_genotyped |
| SL18-UCONN-142-FG3 | not_genotyped | SL20-UCONN-3-MG1 | not_genotyped |
| SL18-UCONN-94-FG3 | not_genotyped | SL18-UCONN-5-MG1 | not_genotyped |

TABLE 3

| Muta- | | protein_ | | | | | gene_ | | |
|---|---|---|---|---|---|---|---|---|---|
| tion | effect | product | CHROM | POS | REF | ALT | ID | Start | End |
| 1 | splice_ | hypothetical | scaffold | 504066 | A | G | gene_ | 503138 | 505839 |
| | acceptor_ | protein | 365 | | | | 20310 | | |
| | variant& | | | | | | | | |
| | intron_ | | | | | | | | |
| | variant | | | | | | | | |
| 2 | frameshift_ | Metallo- | scaffold | 33376166742 | T | TAG | gene_ | 3327359 | 3336806 |
| | variant | dependent | 16 | | | | 3898 | | |
| | | phosphatase | | | | | | | |
| | | like protein | | | | | | | |
| 3 | frameshift_ | hypothetical | scaffold | 507788 | G | GT | gene_ | 506902 | 508472 |
| | variant | protein | 266 | | | | 13011 | | |
| 4 | frameshift_ | hypothetical | scaffold | 10472 | TA | T | gene_ | 8700 | 15670 |
| | variant | protein | 146 | | | | 12208 | | |
| 5 | start_lost | putative | scaffold | 278180 | C | A | gene_ | 260188 | 278182 |
| | | multicopper | 333 | | | | 22011 | | |
| | | oxidase | | | | | | | |
| 6 | frameshift_ | hypothetical | scaffold | 391027 | TGGT | T | gene_ | 390758 | 392133 |
| | variant | protein | 407 | | C | | 19271 | | |
| 7 | frameshift_ | hypothetical | scaffold | 3680820 | GC | G | gene_ | 3680815 | 3708784 |
| | variant | protein | 42 | | | | 4148 | | |
| 8 | frameshift_ | expressed | scaffold | 226297 | C | CTACA | gene_ | 226065 | 226533 |
| | variant | protein | 560 | | | G | 18515 | | |
| 9 | splice_ | hypothetical | scaffold | 249752 | T | C | gene_ | 243769 | 263384 |
| | acceptor_ | protein | 296 | | | | 19506 | | |
| | variant& | | | | | | | | |
| | intron_ | | | | | | | | |
| | variant | | | | | | | | |
| 10 | frameshift_ | hypothetical | scaffold | 2152457 | TGAC | T | gene_ | 2133413 | 2156252 |
| | variant | protein | 39 | | C | | 4387 | | |
| 11 | frameshift_ | hypothetical | scaffold | 2151916 | GT | G | gene_ | 2133413 | 2156252 |
| | variant | protein | 39 | | | | 4387 | | |
| 12 | frameshift_ | acetyltrans- | scaffold | 283178 | GC | G | gene_ | 280424 | 314025 |
| | variant | ferase 1-like | 490 | | | | 20370 | | |
| 13 | frameshift_ | expressed | scaffold | 163030 | C | CGGTA | gene_ | 162909 | 163927 |
| | variant | protein | 424 | | | T | 18887 | | |
| 14 | frameshift_ | Topoisomerase | scaffold | 825179 | AG | A | gene_ | 811520 | 830548 |
| | variant | l-binding | 207 | | | | 14738 | | |
| | | arginine- | | | | | | | |
| | | serine-rich | | | | | | | |
| | | protein | | | | | | | |
| 15 | splice_ | Protein | scaffold | 75723 | C | T | gene_ | 73680 | 75728 |
| | donor_ | required for | 671 | | | | 23147 | | |
| | variant& | meiotic | | | | | | | |
| | intron_ | chromosome | | | | | | | |
| | variant | segregation | | | | | | | |
| 16 | frameshift_ | Meiotic | scaffold | 757237 | C | CG | gene_ | 752975 | 757641 |
| | variant | chromosome | 119 | | | | 8000 | | |
| | | segregation | | | | | | | |
| | | protein | | | | | | | |
| 17 | stop_gained | expressed | scaffold | 226307 | T | A | gene_ | 226065 | 226533 |
| | | protein | 560 | | | | 18515 | | |
| 18 | 3_prime | hypothetical | scaffold | 507350 | G | C | gene_ | 506902 | 508472 |
| | UTR_variant | protein | 266 | | | | 13011 | | |
| 19 | frameshift_ | hypothetical | scaffold | 507788 | G | GTT | gene_ | 506902 | 508472 |
| | variant | protein | 266 | | | | 13011 | | |
| 20 | frameshift_ | hypothetical | scaffold | 507791 | T | TG | gene_ | 506902 | 508472 |
| | variant | protein | 266 | | | | 13011 | | |
| 21 | frameshift_ | Formate- | scaffold | 797 | A | AC | gene_ | 357 | 14690 |
| | variant | tetrahydro- | 302 | | | | 18640 | | |
| | | folate ligase | | | | | | | |
| 22 | stop_gained | Formate- | scaffold | 473 | G | A | gene_ | 357 | 14690 |
| | | tetrahydro- | 302 | | | | 18640 | | |
| | | folate ligase | | | | | | | |
| 23 | splice_ | hypothetical | scaffold | 249751 | C | G | gene_ | 243769 | 263384 |
| | acceptor_ | protein | 296 | | | | 19506 | | |
| | variant& | | | | | | | | |
| | intron_ | | | | | | | | |
| | variant | | | | | | | | |
| 24 | splice_ | hypothetical | scaffold | 249751 | C | T | gene_ | 243769 | 263384 |
| | acceptor_ | protein | 296 | | | | 19506 | | |
| | variant& | | | | | | | | |
| | intron_ | | | | | | | | |
| | variant | | | | | | | | |
| 25 | frameshift_ | Metallo- | scaffold | 3330517 | GC | G | gene_ | 3327359 | 3336806 |
| | variant | dependent | 16 | | | | 3898 | | |
| | | phosphatase- | | | | | | | |
| | | like protein | | | | | | | |
| 26 | frameshift_ | hypothetical | scaffold | 2152457 | TGAC | T | gene_ | 2133413 | 2156252 |
| | variant | protein | 39 | | C | | 4387 | | |
| 27 | frameshift_ | hypothetical | scaffold | 10784 | AG | A | gene_ | 8700 | 15670 |
| | variant | protein | 146 | | | | 12208 | | |
| 28 | frameshift_ | hypothetical | scaffold | 10515 | GTA | G | gene_ | 8700 | 15670 |
| | variant | protein | 146 | | | | 12208 | | |
| 29 | frameshift_ | hypothetical | scaffold | 10732 | ATGC | A | gene_ | 8700 | 15670 |
| | variant | protein | 146 | | TACT | | 12208 | | |
| | | | | | ATTA | | | | |
| | | | | | ATAC | | | | |
| | | | | | CAGG | | | | |
| | | | | | TACG | | | | |
| 30 | frameshift_ | expressed | scaffold | 163030 | C | CGGTA | gene_ | 162909 | 163927 |
| | variant | protein | 424 | | | TGGTA | 18887 | | |
| | | | | | | T | | | |
| 31 | 3_prime_ | expressed | scaffold | 163862 | A | ACTCC | gene_ | 162909 | 163927 |
| | UTR_variant | protein | 424 | | | | 18887 | | |
| 32 | frameshift_ | expressed | scaffold | 163212 | G | GT | gene_ | 162909 | 163927 |
| | variant | protein | 424 | | | | 18887 | | |
| 33 | frameshift_ | expressed | scaffold | 163030 | CGGT | C | gene_ | 162909 | 163927 |
| | variant | protein | 424 | | AT | | 18887 | | |
| 34 | frameshift_ | Topoisomerase | scaffold | 825603 | GGAG | G | gene_ | 811520 | 830548 |
| | variant | l-binding | 207 | | GA | | 14738 | | |
| | | arginine- | | | | | | | |
| | | serine-rich | | | | | | | |
| | | protein | | | | | | | |
| 35 | frameshift_ | Topoisomerase | scaffold | 825601 | GA | G | gene_ | 811520 | 830548 |
| | variant | l-binding | 207 | | | | 14738 | | |
| | | arginine- | | | | | | | |
| | | serine-rich | | | | | | | |
| | | protein | | | | | | | |
| 36 | frameshift_ | Topoisomerase | scaffold | 820414 | C | CAGCG | gene_ | 811520 | 830548 |
| | variant | l-binding | 207 | | | T | 14738 | | |
| | | arginine- | | | | | | | |
| | | serine-rich | | | | | | | |
| | | protein | | | | | | | |
| 37 | frameshift_ | hypothetical | scaffold | 504025 | GGCC | G | gene_ | 503138 | 505839 |
| | variant& | protein | 365 | | TGCT | | 20310 | | |
| | splice_ | | | | CACG | | | | |
| | acceptor_ | | | | GGTC | | | | |
| | variant& | | | | GGGC | | | | |
| | splice | | | | AAAC | | | | |
| | region_ | | | | CTCA | | | | |
| | variant& | | | | CGGG | | | | |
| | intron_ | | | | GCTC | | | | |
| | variant | | | | GGTC | | | | |
| | | | | | CAGT | | | | |
| | | | | | CCGG | | | | |
| | | | | | TCCG | | | | |
| | | | | | A | | | | |
| 38 | frameshift_ | hypothetical | scaffold | 410188 | TAGA | T | gene_ | 406170 | 415376 |
| | variant& | protein | 432 | | CCCT | | 21578 | | |
| | splice_ | | | | CGCA | | | | |
| | acceptor_ | | | | TCCC | | | | |
| | variant& | | | | TACA | | | | |
| | splice | | | | ATGC | | | | |
| | region_ | | | | CGGG | | | | |
| | variant& | | | | ACGG | | | | |
| | intron_ | | | | AG | | | | |
| | variant | | | | | | | | |
| 39 | stop_gained | Protein | scaffold | 74169 | G | C | gene_ | 73680 | 75728 |
| | | required for | 671 | | | | 23147 | | |
| | | meiotic | | | | | | | |
| | | chromosome | | | | | | | |
| | | segregation | | | | | | | |
| 40 | frameshift_ | hypothetical | scaffold | 4348 | AACC | A | gene_ | 3019 | 4600 |
| | variant | protein | 877 | | G | | 24927 | | |
| 41 | frameshift_ | hypothetical | scaffold | 3694421 | T | TC | gene_ | 3680815 | 3708784 |
| | variant | protein | 42 | | | | 4148 | | |
| 42 | frameshift_ | hypothetical | scaffold | 3694454 | T | TC | gene_ | 3680815 | 3708784 |
| | variant | protein | 42 | | | | 4148 | | |
| 43 | frameshift_ | hypothetical | scaffold | 2459722 | C | CA | gene_ | 2454377 | 2460388 |
| | variant | protein | 17 | | | | 5208 | | |
| 44 | frameshift_ | hypothetical | scaffold | 2459381 | CT | C | gene_ | 2454377 | 2460388 |
| | variant | protein | 17 | | | | 5208 | | |
| 45 | frameshift_ | hypothetical | scaffold | 2459713 | CCCT | C | gene_ | 2454377 | 2460388 |
| | variant | protein | 17 | | A | | 5208 | | |
| 46 | frameshift_ | Meiotic | scaffold | 757424 | TG | T | gene_ | 752975 | |
| | variant | chromosome | 119 | | | | 8000 | | 757641 |
| | | segregation | | | | | | | |
| | | protein | | | | | | | |
| 47 | frameshift_ | Protein- | scaffold | 225780 | AC | A | gene_ | 225771 | 226651 |
| | variant | tyrosine | 161 | | | | 9676 | | |
| | | phosphatase- | | | | | | | |
| | | like protein | | | | | | | |

TABLE 4

| Mutation | transcript_ID | protein_ID | protein_product | BLAST or annotation information evidencing relevant phenotype potential |
|---|---|---|---|---|
| 1 | 3526679 | 3525423 | hypothetical | Rad50_PGA_scaffold16_19_contigs_length_23964549:15841555- |
| | | | protein | 15866490.p12; |
| | | | | Rad50_PGA_scaffold16_19_contigs_length_23964549:15841555- |
| | | | | 15866490.p12; |
| | | | | Rad50_PGA_scaffold16_19_contigs_length_23964549:15841555- |
| | | | | 15866490.p12; |
| | | | | Rad50_PGA_scaffold16_19_contigs_length_23964549:15841555- |
| | | | | 15866490.p12 |
| 2 | 2335174 | 2333918 | Metallo- | Rad50_PGA_scaffold16_19_contigs_length_23964549:15841555- |
| | | | dependent | 15866490.p16; |
| | | | phosphatase- | Rad50_PGA_scaffold16_19_contigs_length_23964549:15841555- |
| | | | like protein | 15866490.p10; |
| | | | | Rad50_PGA_scaffold16_19_contigs_length_23964549:15841555- |
| | | | | 15866490.p8; |
| | | | | Mre11_PGA_scaffold23_18_contigs_length_14937655:8345170- |
| | | | | 8354106.p11 |
| 3 | 2972194 | 2970938 | hypothetical | Rad50_PGA_scaffold16_19_contigs_length_23964549:15841555- |
| | | | protein | 15866490.p8; |
| | | | | Rad50_PGA_scaffold16_19_contigs_length_23964549:15841555- |
| | | | | 15866490.p16; |
| | | | | Rad50_PGA_scaffold16_19_contigs_length_23964549:15841555- |
| | | | | 15866490.p10 |
| 4 | 2780261 | 2779005 | hypothetical | Rad50_PGA_scaffold16_19_contigs_length_23964549:15841555- |
| | | | protein | 15866490.p10; |
| | | | | Rad50_PGA_scaffold16_19_contigs_length_23964549:15841555- |
| | | | | 15866490.p8; |
| | | | | Rad50_PGA_scaffold16_19_contigs_length_23964549:15841555- |
| | | | | 15866490.p16 |
| 5 | 3461005 | 3459749 | putative | Rad50_PGA_scaffold16_19_contigs_length_23964549:15841555- |
| | | | multicopper | 15866490.p4; |
| | | | oxidase | Rad50_PGA_scaffold16_19_contigs_length_23964549:15841555- |
| | | | | 15866490.p16 |
| 6 | 3530267 | 3529011 | hypothetical | TOPVIA_PGA_scaffold25_21_contigs_length_13377422:4338542- |
| | | | protein | 4348738.p3; |
| | | | | TOPVIA_PGA_Scaffold25_21_contigs_length_13377422:4338542- |
| | | | | 4348738.p3 |
| 7 | 3385351 | 3384095 | hypothetical | Rck_PGA_scaffold8_83_contigs_length_51047780:43857782- |
| | | | protein | 43863559.p6 |
| 8 | 3176610 | 3175354 | expressed | Rad50_PGA_scaffold16_19_contigs_length_23964549:15841555- |
| | | | protein | 15866490.p12 |
| 9 | 3480387 | 3479131 | hypothetical | Rad50_PGA_scaffold16_19_contigs_length_23964549:15841555- |
| | | | protein | 15866490.p28 |
| 10 | 2203251 | 2201995 | hypothetical | TOPVIA_PGA_scaffold25_21_contigs_length_13377422:4338542- |
| | | | protein | 4348738.p3 |
| 11 | 2203251 | 2201995 | hypothetical | TOPVIA_PGA_scaffold25_21_contigs_length_13377422:4338542- |
| | | | protein | 4348738.p3 |
| 12 | 3393522 | 3392266 | acetyltransferase | Rad50_PGA_scaffold16_19_contigs_length_23964549:15841555- |
| | | | 1-like | 15866490.p4 |
| 13 | 3106376 | 3105120 | expressed | Rad50_PGA_scaffold16_19_contigs_length_23964549:15841555- |
| | | | protein | 15866490.p12 |
| 14 | 3381176 | 3379920 | Topoisomerase | JGI_annot |
| | | | I-binding | |
| | | | arginine- | |
| | | | serine-rich | |
| | | | protein | |
| 15 | 3417497 | 3416241 | Protein | JGI_annot |
| | | | required for | |
| | | | meiotic | |
| | | | chromosome | |
| | | | segregation | |
| 16 | 3475393 | 3474137 | Meiotic | JGI_annot |
| | | | chromosome | |
| | | | segregation | |
| | | | protein | |
| 17 | 3176610 | 3175354 | expressed | Rad50_PGA_scaffold16_19_contigs_length_23964549:15841555- |
| | | | protein | 15866490.p12 |
| 18 | 2972194 | 2970938 | hypothetical | Rad50_PGA_scaffold16_19_contigs_length_23964549:15841555- |
| | | | protein | 15866490.p8; |
| | | | | Rad50_PGA_scaffold16_19_contigs_length_23964549:15841555- |
| | | | | 15866490.p16; |
| | | | | Rad50_PGA_scaffold16_19_contigs_length_23964549:15841555- |
| | | | | 15866490.p10 |
| 19 | 2972194 | 2970938 | hypothetical | Rad50_PGA_scaffold16_19_contigs_length_23964549:15841555- |
| | | | protein | 15866490.p8; |
| | | | | Rad50_PGA_scaffold16_19_contigs_length_23964549:15841555- |
| | | | | 15866490.p16; |
| | | | | Rad50_PGA_scaffold16_19_contigs_length_23964549:15841555- |
| | | | | 15866490.p10 |
| 20 | 2972194 | 2970938 | hypothetical | Rad50_PGA_scaffold16_19_contigs_length_23964549:15841555- |
| | | | protein | 15866490.p8; |
| | | | | Rad50_PGA_scaffold16_19_contigs_length_23964549:15841555- |
| | | | | 15866490.p16; |
| | | | | Rad50_PGA_scaffold16_19_contigs_length_23964549:15841555- |
| | | | | 15866490.p10 |
| 21 | 3381660 | 3380404 | Formate- | Rad50_PGA_scaffold16_19_contigs_length_23964549:15841555- |
| | | | tetrahydrofolate | 15866490.p23 |
| | | | ligase | |
| 22 | 3381660 | 3380404 | Formate- | Rad50_PGA_scaffold16_19_contigs_length_23964549:15841555- |
| | | | tetrahydrofolate | 15866490.p23 |
| | | | ligase | |
| 23 | 3480387 | 3479131 | hypothetical | Rad50_PGA_scaffold16_19_contigs_length_23964549:15841555- |
| | | | protein | 15866490.p28 |
| 24 | 3480387 | 3479131 | hypothetical | Rad50_PGA_scaffold16_19_contigs_length_23964549:15841555- |
| | | | protein | 15866490.p28 |
| 25 | 2335174 | 2333918 | Metallo- | Rad50_PGA_scaffold16_19_contigs_length_23964549:15841555- |
| | | | dependent | 15866490.p16; |
| | | | phosphatase- | Rad50_PGA_scaffold16_19_contigs_length_23964549:15841555- |
| | | | like protein | 15866490.p10; |
| | | | | Rad50_PGA_scaffold16_19_contigs_ length_23964549:15841555- |
| | | | | 15866490.p8; |
| | | | | Mre11_PGA_scaffold23_18_contigs_length_14937655:8345170- |
| | | | | 8354106.p11 |
| 26 | 2203251 | 2201995 | hypothetical | TOPVIA_PGA_scaffold25_21_contigs_length_13377422:4338542- |
| | | | protein | 4348738.p3 |
| 27 | 2780261 | 2779005 | hypothetical | Rad50_PGA_scaffold16_19_contigs_length_23964549:15841555- |
| | | | protein | 15866490.p10; |
| | | | | Rad50_PGA_scaffold16_19_contigs_length_23964549:15841555- |
| | | | | 15866490.p8; |
| | | | | Rad50_PGA_scaffold16_19_contigs_length_23964549:15841555- |
| | | | | 15866490.p16 |
| 28 | 2780261 | 2779005 | hypothetical | Rad50_PGA_scaffold16_19_contigs_length_23964549:15841555- |
| | | | protein | 15866490.p10; |
| | | | | Rad50_PGA_scaffold16_19_contigs_length_23964549:15841555- |
| | | | | 15866490.p8; |
| | | | | Rad50_PGA_scaffold16_19_contigs_length_23964549:15841555- |
| | | | | 15866490.p16 |
| 29 | 2780261 | 2779005 | hypothetical | Rad50_PGA_scaffold16_19_contigs_length_23964549:15841555- |
| | | | protein | 15866490.p10; |
| | | | | Rad50_PGA_scaffold16_19_contigs_length_23964549:15841555- |
| | | | | 15866490.p8; |
| | | | | Rad50_PGA_scaffold16_19_contigs_length_23964549:15841555- |
| | | | | 15866490.p16 |
| 30 | 3106376 | 3105120 | expressed | Rad50_PGA_scaffold16_19_contigs_length_23964549:15841555- |
| | | | protein | 15866490.p12 |
| 31 | 3106376 | 3105120 | expressed | Rad50_PGA_scaffold16_19_contigs_length_23964549:15841555- |
| | | | protein | 15866490.p12 |
| 32 | 3106376 | 3105120 | expressed | Rad50_PGA_scaffold16_19_contigs_length_23964549:15841555- |
| | | | protein | 15866490.p12 |
| 33 | 3106376 | 3105120 | expressed | Rad50_PGA_scaffold16_19_contigs_length_23964549:15841555- |
| | | | protein | 15866490.p12 |
| 34 | 3381176 | 3379920 | Topoisomerase | JGI_annot |
| | | | I-binding | |
| | | | arginine- | |
| | | | serine-rich | |
| | | | protein | |
| 35 | 3381176 | 3379920 | Topoisomerase | JGI_annot |
| | | | I-binding | |
| | | | arginine- | |
| | | | serine-rich | |
| | | | protein | |
| 36 | 3381176 | 3379920 | Topoisomerase | JGI_annot |
| | | | I-binding | |
| | | | arginine- | |
| | | | serine-rich | |
| | | | protein | |
| 37 | 3526679 | 3525423 | hypothetical | Rad50_PGA_scaffold16_19_contigs_length_23964549:15841555- |
| | | | protein | 15866490.p12; |
| | | | | Rad50_PGA_scaffold16_19_contigs_length_23964549:15841555- |
| | | | | 15866490.p12; |
| | | | | Rad50_PGA_scaffold16_19_contigs_length_23964549:15841555- |
| | | | | 15866490.p12; |
| | | | | Rad50_PGA_scaffold16_19_contigs_length_23964549:15841555- |
| | | | | 15866490.p12 |
| 38 | 3532687 | 3531431 | hypothetical | Rad50_PGA_scaffold16_19_contigs_length_23964549:15841555- |
| | | | protein | 15866490.p22; |
| | | | | Mre11_PGA_scaffold23_18_contigs_length_14937655:8345170- |
| | | | | 8354106.p6; |
| | | | | Rad50_PGA_scaffold16_19_contigs_length_23964549:15841555- |
| | | | | 15866490.p28 |
| 39 | 3417497 | 3416241 | Protein | JGI_annot |
| | | | required for | |
| | | | meiotic | |
| | | | chromosome | |
| | | | segregation | |
| 40 | 3552692 | 3551436 | hypothetical | TOPVIA_PGA_scaffold25_21_contigs_length_13377422:4338542- |
| | | | protein | 4348738.p3; |
| | | | | TOPVIA_PGA_Scaffold25_21_contigs_length_13377422:4338542- |
| | | | | 4348738.p3; |
| | | | | TOPVIA_PGA_scaffold25_21_contigs_length_13377422:4338542- |
| | | | | 4348738.p3; |
| | | | | TOPVIA_PGA_Scaffold25_21_contigs_length_13377422:4338542- |
| | | | | 4348738.p3 |
| 41 | 3385351 | 3384095 | hypothetical | Rck_PGA_scaffold8_83_contigs_length_51047780:43857782- |
| | | | protein | 43863559.p6 |
| 42 | 3385351 | 3384095 | hypothetical | Rck_PGA_scaffold8_83_contigs_length_51047780:43857782- |
| | | | protein | 43863559.p6 |
| 43 | 2342182 | 2340926 | hypothetical | Rad50_PGA_scaffold16_19_contigs_length_23964549:15841555- |
| | | | protein | 15866490.p22; |
| | | | | Mre11_PGA_scaffold23_18_contigs_length_14937655:8345170- |
| | | | | 8354106.p6; |
| | | | | Rad50_PGA_scaffold16_ 19_contigs_length_23964549:15841555- |
| | | | | 15866490.p28 |
| 44 | 2342182 | 2340926 | hypothetical | Rad50_PGA_scaffold16_19_contigs_length_23964549:15841555- |
| | | | protein | 15866490.p22; |
| | | | | Mre11_PGA_scaffold23_18_contigs_length_14937655:8345170- |
| | | | | 8354106.p6; |
| | | | | Rad50_PGA_scaffold16_19_contigs_length_23964549:15841555- |
| | | | | 15866490.p28 |
| 45 | 2342182 | 2340926 | hypothetical | Rad50_PGA_scaffold16_19_contigs_length_23964549:15841555- |
| | | | protein | 15866490.p22; |
| | | | | Mre11_PGA_scaffold23_18_contigs_1ength_14937655:8345170- |
| | | | | 8354106.p6; |
| | | | | Rad50_PGA_scaffold16_19_contigs_length_23964549:15841555- |
| | | | | 15866490.p28 |
| 46 | 3475393 | 3474137 | Meiotic | JGI_annot |
| | | | chromosome | |
| | | | segregation | |
| | | | protein | |
| 47 | 2814348 | 2813092 | Protein- | Rad50_PGA_scaffold16_19_contigs_length_23964549:15841555- |
| | | | tyrosine | 15866490.p8 |
| | | | phosphatase- | |
| | | | like protein | |

TABLE 5
The cross of SL.OI.11.MG.2 and SL-CC-1-GF-2 was highly effective, and the
cross of SA.CB.4.FG.3 and SL-JS-19-MG-1 was also particularly effective.

| | | | | SA-CB- | SL-LL- | SL-JS- | SL-NL- | SL-LL- | SL-SF- | SL-CC- |
|---|---|---|---|---|---|---|---|---|---|---|
| Mutation | SA.CB.4.FG.3 | SL.JS.19.MG.2 | SL.OI.11.MG.2 | 7-MG-3 | 9-MG-4 | 19-MG-1 | 2-MG-1 | 9-MG-4 | 13-FG-2 | 1-FG-2 |
| 1 | no | NA | NA | no | no | yes | no | no | no | no |
| 2 | yes | no | NA | yes | yes | no | no | no | no | yes |
| 3 | yes | no | no | yes | yes | yes | no | no | no | no |
| 4 | yes | NA | NA | yes | no | yes | no | no | no | no |
| 5 | no | yes | NA | | | no | | | | |
| 6 | yes | no | no | | | no | | | | |
| 7 | NA | NA | yes | yes | no | no | no | no | no | no |
| 8 | yes | yes | NA | yes | yes | yes | yes | no | no | no |
| 9 | yes | NA | yes | no | yes | yes | no | no | yes | no |
| 10 | no | NA | NA | no | no | yes | yes | no | no | yes |
| 11 | yes | no | no | no | no | yes | yes | no | no | yes |
| 12 | yes | NA | NA | yes | no | no | no | no | no | no |
| 13 | yes | yes | no | no | yes | yes | no | no | no | no |
| 14 | no | no | no | no | no | yes | no | no | no | no |
| 15 | no | no | yes | no | no | no | no | no | yes | no |
| 16 | yes | yes | NA | no | no | no | yes | no | no | no |
| 17 | | | | yes | yes | yes | yes | no | no | no |
| 18 | | | | yes | yes | yes | no | no | no | no |
| 19 | | | | yes | yes | yes | no | no | no | no |
| 20 | | | | yes | yes | yes | no | no | no | no |
| 21 | | | | yes | yes | no | yes | no | no | no |
| 22 | | | | yes | yes | no | yes | no | no | no |
| 23 | | | | no | yes | yes | no | no | yes | no |
| 24 | | | | no | yes | yes | no | no | yes | no |
| 25 | | | | yes | yes | no | no | no | no | yes |
| 26 | | | | no | no | yes | yes | no | no | yes |
| 27 | | | | yes | no | yes | no | no | no | no |
| 28 | | | | yes | no | yes | no | no | no | no |
| 29 | | | | yes | no | yes | no | no | no | no |
| 30 | | | | no | yes | yes | no | no | no | no |
| 31 | | | | no | yes | yes | no | no | no | no |
| 32 | | | | no | yes | yes | no | no | no | no |
| 33 | | | | no | yes | yes | no | no | no | no |
| 34 | | | | no | no | yes | no | no | no | no |
| 35 | | | | no | no | yes | no | no | no | no |
| 36 | | | | no | no | yes | no | no | no | no |
| 37 | | | | no | no | yes | no | no | no | no |
| 38 | | | | no | yes | no | no | no | no | no |
| 39 | | | | no | no | no | no | no | yes | no |
| 40 | | | | no | yes | no | no | no | no | no |
| 41 | | | | yes | no | no | no | no | no | no |
| 42 | | | | yes | no | no | no | no | no | no |
| 43 | | | | no | no | no | yes | no | no | no |
| 44 | | | | no | no | no | yes | no | no | no |
| 45 | | | | no | no | no | yes | no | no | no |
| 46 | | | | no | no | no | yes | no | no | no |
| 47 | | | | yes | no | no | no | no | no | no |

TABLE 6
Mutations 2 and 9 are particularly effective.

| Mutation | Further BLAST or annotation information evidencing relevant phenotype potential |
|---|---|
| 1 | Rad50_PGA_scaffold16_19_contigs_length_23964549:15841555-15866490.p12; |
| | Rad50_PGA_scaffold16_19_contigs_length_23964549:15841555-15866490.p12; |
| | Rad50_PGA_scaffold16_19_contigs_length_23964549:15841555-15866490.p12; |
| | Rad50_PGA_scaffold16_19_contigs_length_23964549:15841555-15866490.p12 |
| 2 | Rad50_PGA_scaffold16_19_contigs_length_23964549:15841555-15866490.p16; |
| | Rad50_PGA_scaffold16_19_contigs_length_23964549:15841555-15866490.p10; |
| | Rad50_PGA_scaffold16_19_contigs_length_23964549:15841555-15866490.p8; |
| | Mre11_PGA_scaffold23_18_contigs_length_14937655:8345170-8354106.p11 |
| 3 | Rad50_PGA_scaffold16_19_contigs_length_23964549:15841555-15866490.p8; |
| | Rad50_PGA_scaffold16_19_contigs_length_23964549:15841555-15866490.p16; |
| | Rad50_PGA_scaffold16_19_contigs_length_23964549:15841555-15866490.p10 |
| 4 | Rad50_PGA_scaffold16_19_contigs_length_23964549:15841555-15866490.p10; |
| | Rad50_PGA_scaffold16_19_contigs_length_23964549:15841555-15866490.p8; |
| | Rad50_PGA_scaffold16_19_contigs_length_23964549:15841555-15866490.p16 |
| 5 | Rad50_PGA_scaffold16_19_contigs_length_23964549:15841555-15866490.p4; |
| | Rad50_PGA_scaffold16_19_contigs_length_23964549:15841555-15866490.p16 |
| 6 | TOPVIA_PGA_scaffold25_21_contigs_length_13377422:4338542-4348738.p3; |
| | TOPVIA_PGA_scaffold25_21_contigs_length_13377422:4338542-4348738.p3 |
| 7 | Rck_PGA_scaffold8_83_contigs_length_51047780:43857782-43863559.p6 |
| 8 | Rad50_PGA_scaffold16_19_contigs_length_23964549:15841555-15866490.p12 |
| 9 | Rad50_PGA_scaffold16_19_contigs_length_23964549:15841555-15866490.p28 |
| 10 | TOPVIA_PGA_scaffold25_21_contigs_length_13377422:4338542-4348738.p3 |
| 11 | TOPVIA_PGA_scaffold25_21_contigs_length_13377422:4338542-4348738.p3 |
| 12 | Rad50_PGA_scaffold16_19_contigs_length_23964549:15841555-15866490.p4 |
| 13 | Rad50_PGA_scaffold16_19_contigs_length_23964549:15841555-15866490.p12 |
| 14 | JGI_annot |
| 15 | JGI_annot |
| 16 | JGI_annot |
| 17 | Rad50_PGA_scaffold16_19_contigs_length_23964549:15841555-15866490.p12 |
| 18 | Rad50_PGA_scaffold16_19_contigs_length_23964549:15841555-15866490.p8; |
| | Rad50_PGA_scaffold16_19_contigs_length_23964549:15841555-15866490.p16; |
| | Rad50_PGA_scaffold16_19_contigs_length_23964549:15841555-15866490.p10 |
| 19 | Rad50_PGA_scaffold16_19_contigs_length_23964549:15841555-15866490.p8; |
| | Rad50_PGA_scaffold16_19_contigs_length_23964549:15841555-15866490.p16; |
| | Rad50_PGA_scaffold16_19_contigs_length_23964549:15841555-15866490.p10 |
| 20 | Rad50_PGA_scaffold16_19_contigs_length_23964549:15841555-15866490.p8; |
| | Rad50_PGA_scaffold16_19_contigs_length_23964549:15841555-15866490.p16; |
| | Rad50_PGA_scaffold16_19_contigs_length_23964549:15841555-15866490.p10 |
| 21 | Rad50_PGA_scaffold16_19_contigs_length_23964549:15841555-15866490.p23 |
| 22 | Rad50_PGA_scaffold16_19_contigs_length_23964549:15841555-15866490.p23 |
| 23 | Rad50_PGA_scaffold16_19_contigs_length_23964549:15841555-15866490.p28 |
| 24 | Rad50_PGA_scaffold16_19_contigs_length_23964549:15841555-15866490.p28 |
| 25 | Rad50_PGA_scaffold16_19_contigs_length_23964549:15841555-15866490.p16; |
| | Rad50_PGA_scaffold16_19_contigs_length_23964549:15841555-15866490.p10; |
| | Rad50_PGA_scaffold16_19_contigs_length_23964549:15841555-15866490.p8; |
| | Mre11_PGA_scaffold23_18_contigs_length_14937655:8345170-8354106.p11 |
| 26 | TOPVIA_PGA_scaffold25_21_contigs_length_13377422:4338542-4348738.p3 |
| 27 | Rad50_PGA_scaffold16_19_contigs_length_23964549:15841555-15866490.p10; |
| | Rad50_PGA_scaffold16_19_contigs_length_23964549:15841555-15866490.p8; |
| | Rad50_PGA_scaffold16_19_contigs_length_23964549:15841555-15866490.p16 |
| 28 | Rad50_PGA_scaffold16_19_contigs_length_23964549:15841555-15866490.p10; |
| | Rad50_PGA_scaffold16_19_contigs_length_23964549:15841555-15866490.p8; |
| | Rad50_PGA_scaffold16_19_contigs_length_23964549:15841555-15866490.p16 |
| 29 | Rad50_PGA_scaffold16_19_contigs_length_23964549:15841555-15866490.p10; |
| | Rad50_PGA_scaffold16_19_contigs_length_23964549:15841555-15866490.p8; |
| | Rad50_PGA_scaffold16_19_contigs_length_23964549:15841555-15866490.p16 |
| 30 | Rad50_PGA_scaffold16_19_contigs_length_23964549:15841555-15866490.p12 |
| 31 | Rad50_PGA_scaffold16_19_contigs_length_23964549:15841555-15866490.p12 |
| 32 | Rad50_PGA_scaffold16_19_contigs_length_23964549:15841555-15866490.p12 |
| 33 | Rad50_PGA_scaffold16_19_contigs_length_23964549:15841555-15866490.p12 |
| 34 | JGI_annot |
| 35 | JGI_annot |
| 36 | JGI_annot |
| 37 | Rad50_PGA_scaffold16_19_contigs_length_23964549:15841555-15866490.p12; |
| | Rad50_PGA_scaffold16_19_contigs_length_23964549:15841555-15866490.p12; |
| | Rad50_PGA_scaffold16_19_contigs_length_23964549:15841555-15866490.p12; |
| | Rad50_PGA_scaffold16_19_contigs_length_23964549:15841555-15866490.p12 |
| 38 | Rad50_PGA_scaffold16_19_contigs_length_23964549:15841555-15866490.p22; |
| | Mre11_PGA_scaffold23_18_contigs_length_14937655:8345170-8354106.p6; |
| | Rad50_PGA_scaffold16_19_contigs_length_23964549:15841555-15866490.p28 |
| 39 | JGI_annot |
| 40 | TOPVIA_PGA_scaffold25_21_contigs_length_13377422:4338542-4348738.p3; |
| | TOPVIA_PGA_scaffold25_21_contigs_length_13377422:4338542-4348738.p3; |
| | TOPVIA_PGA_scaffold25_21_contigs_length_13377422:4338542-4348738.p3; |
| | TOPVIA_PGA_scaffold25_21_contigs_length_13377422:4338542-4348738.p3 |
| 41 | Rck_PGA_scaffold8_83_contigs_length_51047780:43857782-43863559.p6 |
| 42 | Rck_PGA_scaffold8_83_contigs_length_51047780:43857782-43863559.p6 |
| 43 | Rad50_PGA_scaffold16_19_contigs_length_23964549:15841555-15866490.p22; |
| | Mre11_PGA_scaffold23_18_contigs_length_14937655:8345170-8354106.p6; |
| | Rad50_PGA_scaffold16_19_contigs_length_23964549:15841555-15866490.p28 |
| 44 | Rad50_PGA_scaffold16_19_contigs_length_23964549:15841555-15866490.p22; |
| | Mre11_PGA_scaffold23_18_contigs_length_14937655:8345170-8354106.p6; |
| | Rad50_PGA_scaffold16_19_contigs_length_23964549:15841555-15866490.p28 |
| 45 | Rad50_PGA_scaffold16_19_contigs_length_23964549:15841555-15866490.p22; |
| | Mre11_PGA_scaffold23_18_contigs_length_14937655:8345170-8354106.p6; |
| | Rad50_PGA_scaffold16_19_contigs_length_23964549:15841555-15866490.p28 |
| 46 | JGI_annot |
| 47 | Rad50_PGA_scaffold16_19_contigs_length_23964549:15841555-15866490.p8 |

TABLE 7

| Mutation | gene | effect | CHROM | POS | REF | ALT |
|---|---|---|---|---|---|---|
| 48 | OUROBOROS | splice_acceptor_variant | scaffold | 469438 | C | T |
| | | &intron_variant | 287 | | | |
| 49 | SAMSARA | frameshift_variant | scaffold | 1047296 | ACT | A |
| | | | 69 | | GGC | |
| | | | | | GG | |
| 50 | SAMSARA | stop_gained | scaffold | 1048797 | G | T |
| | | | 69 | | | |
| 51 | SAMSARA | splice_acceptor_variant | scaffold | 1016260 | G | A |
| | | &intron_variant | 69 | | | |

### Example 1

The feasibility, benefits, risks, and consequences of using sporeless sporophytes to aid the upscaling of kelp cultivation in, e.g., Europe, North America, Asia, Australia, New Zealand and Africa, as well as the barriers that currently exist and how these may be overcome, are reviewed here. Taking environmental, industrial, and legal factors into account, the use of domesticated sporeless sporophytes as an asset to facilitate upscaling kelp cultivation without potentially impacting wild genetic diversity is considered.

### INTRODUCTION

The human population is projected to hit roughly 10 billion by 2050 (UN 2017) and, accordingly, global food production will need to increase by 50-60%. With the additional threat of climate change, there is an urgent need for a worldwide transition from energy-, freshwater-, and arable land-intensive crops to low-carbon crops like farmed seaweed to provide, for example, food, feedstocks for animal feeds, biofuels, and bioplastics. Seaweed farmed as a crop has the potential to facilitate the sustainable, economic, and ecological transition to a circular economy in food and energy production (Aiking and de Boer 2020, van den Burg et al. 2021). With oceans covering 71% of the Earth's surface but accounting for just 2% of global food production (Van Zanten et al. 2019), there is a huge potential to use this vast space. Only a portion of this space would be needed to grow seaweed that is rich in essential nutrients, minerals, carbohydrates, and proteins and which can be used as functional foods or feedstocks for further processing. However, seaweed cultivation is in its early stages in Europe and North America with many scientific and technological hurdles yet to be surmounted (Kim et al. 2019; Hwang et al. 2022; Campbell et al. 2019). Increasing the size and scale of seaweed farming presents many challenges and opportunities. Here, we focus on the opportunity to begin selectively breeding economically important kelp species and the challenge of gaining social licence for those efforts whilst protecting the wild genetic diversity upon which selective breeding is based. Creation of sporeless sporophytes of selectively bred kelps is deemed a key enabling technology for upscaling kelp farming. The term sporeless is used herein to indicate infertile, sterile individuals not capable of reproducing. Kelp seaweed species (Laminariales) are used as an exemplar herein as they have high potential for larger scale cultivation. Sporeless is defined as the inability of sporophytes to produce reproductive sorus tissues with meiospores or produce normal meiospores that can develop into fertile gametophytes. Whilst not anatomically correct, with lay audiences this is equated to the term “seedless” which is generally understood.

### Kelp Cultivation

For seaweed cultivation to reach its socio-economic and market potential, yields need to be increased, for which research and innovation in selective breeding (Charrier et al. 2015; Kim et al. 2019; Hwang et al. 2002; and Liu et al. 2017) and biorefinery processes (Patyshakuliyeva et al. 2019; Torres et al. 2019) is urgently needed (Buschmann et al. 2017; Bak et al. 2018). In 2018, 32.4 million tonnes of aquatic algae were produced globally, of which 97.1% was cultivated seaweed. Most of the cultivated seaweed is produced in East and Southeast Asia, whilst the majority of seaweed produced in Europe and North America is still wild harvested (FAO 2020). Increasing production via wild harvesting risks overexploiting wild seaweed populations, which is neither ecologically sustainable nor financially viable. To meet predicted future demands for seaweed and seaweed-derived products, scaling up ocean farming will be necessary. However, scaling up seaweed cultivation poses logistical challenges, requiring profitable practices whilst minimising any negative impacts on marine resources (Loureiro et al. 2015, Grebe et al. 2019, Visch et al. 2020). Selective breeding techniques capable of cultivating superior strains will be required to achieveenable the quality and quantity of cultivated seaweed necessary to be competitive with other similar products, for example, high-performing kelp strains from Asia, other seafood-based products and, more broadly, with terrestrial crops which have been selectively bred for hundreds of years.

### Selective Breeding

Presently, most seaweed farms in Europe and North America are growing kelp species cultivated from wild spores (e.g., meiospores) or “seed” collected from local kelp blades. This is the traditional and simplest way and, if enough blades are sampled, it ensures that farmed kelp resembles the local wild kelp, thereby reducing the risk of any potential genetic impacts that could be caused by farmed and wild kelp interbreeding in the environment. This is often the approach preferred by regulating marine resource managers who are responsible for protecting wild kelp. Regulators have codified concerns about alien and non-local aquacultured species introductions in European environmental legislation, e.g., Council Regulation EC No. 708/2007 of 11 Jun. 2007 (Barbier et al. 2019),) and stipulated where and how many parents must contribute when breeding seaweed (State of Alaska 2022). However, restricting cultivation to local varieties inhibits the productive and profitable upscaling of kelp cultivation to the degree achieved with selectively improved kelp in Asia (Hu et al. 2021; Hwang et al. 2022). Whilst selective breeding on a research scale (e.g., one-meter plots per MARINER projects; Umanzor et al. 2021; Li et al. 2022) has not posed a risk to wild kelp, it is prudent to be proactive about ensuring that the larger selectively-bred kelp farms of the future do not overwhelm and irreversibly genetically alter wild kelp populations.

We propose herein the use of sporeless or seedless individuals in kelp to induce reproductive sterility (Goecke et al. 2020). Without wishing to be bound by theory, we contemplate at least two alternative ways of producing sporeless individuals; by producing polyploids or by selecting naturally occurring sporeless mutants from the wild population and breeding them such that sterility becomes genetically dominant. Alternatively, there are other ways of reducing or preventing reproduction between farmed and wild populations, for example, by selecting for delayed reproduction in farmed strains to occur only after the normal harvest season, or by locating farms far from natural kelp beds (offshore) or outside of their natural distribution in regions where kelp may be thermally tolerant in the winter but not in the summer and thus cannot naturally complete their lifecycle. It is contemplated herein that using sporeless individuals is the most promising route towards upscaling sustainable kelp cultivation for reasons further described below.

### Lifecycle of Kelps

The kelp life cycle differs from that of higher plants in that the microscopic gametophytic life stage of kelp is physically independent from the macroscopic sporophytic stage. This adaptation can be very useful when domesticating and cultivating kelp since gametophytes can be propagated and maintained as parental material in labs or nurseries for years. If desirable traits can be identified and selectively bred, clonal lines of a favoured genotype can be produced and maintained in a vegetative state to be retrieved and fertilized as required (Ebbing et al. 2021). Selective breeding can be used to produce higher yields or any other desirable trait beneficial for farming and the end product. Results from the MARINER program have demonstrated improvements in selectively bred varieties that amount to twice the typical commercial yields and up to 28 kg m⁻¹ wet weight and 4 kg m⁻¹ dry weight (Li et al. 2022). The ability to safely cultivate selectively bred, non-local and potentially non-native kelp without genetic risk to the environment would transform kelp aquaculture in, e.g., Europe, North America, South America, Asia, Australia, New Zealand, and Africa, by allowing expansion of and increasing the efficiency and reliability of the industry, producing knock-on effects on other aquaculture industries, economies, diets, and innovation using seaweed as a resource.

### Sporeless Kelp Sporophytes

There is potential for the infertility to be achieved in kelp through the production of triploid sporophytes which cannot successfully undergo meiosis to produce viable haploid gametophytes and are thereby infertile. The independent life stages of the kelp gametophyte and sporophyte means that it is possible to cross one haploid and one diploid gametophyte to form a triploid sporophyte.

Homozygous diploid kelp gametophytes can be produced chemically by applying chromosome doubling agents to a haploid gametophyte, such as oryzalin or colchicine (Chauvin et al. 2003). Of the two, oryzalin has been shown to be the more efficient chromosome doubling agent and less likely to cause development of chimaeras than colchicine (Sree Ramulu et al. 1991; Bouvier et al. 1994; Chauvin et al. 2003). Diploid gametophytes can also occur naturally, and it has been suggested that higher environmental stressors increase the occurrence of diploid gametophytes (Oppliger et al. 2014). It can be determined whether these conditions could be artificially replicated in kelp to induce higher numbers of diploid gametophytes and what number could be obtained in this way. Diploid gametophytes can be identified from the population by screening using flow cytometry (Augyte et al. 2022).

The diploid gametophytes can be propagated and maintained as separately contained male and female gametophyte monoclonal cultures in a vegetative state (Lüning and Neushul 1978). Thus, each culture container propagates a single genotype. Gametophytes from these cultures can be used for fertilization and production of triploid sporophytes by crossbreeding select genotypes of the male and female gametophytes. Such cultures can be maintained for years (Barrento et al. 2016; Wade et al. 2020) Macroalgal germplasm banking for conservation, food security, and industry. PLOS Biol 18 (2): e3000641, produce genetically consistent sporophytes, and breeding would no longer be subject to natural seasonal limitations. Desired phenotypes can also be selectively bred and kept in the culture. This process can be scaled up to store large numbers of gametophytes to supply genetically controlled and bio-secure breeding material to commercial kelp growers. Gametophytes can also be stored long term by cryopreservation (Visch et al. 2019; Yang et al. 2022 Germplasm Cryopreservation of Macroalgae for Aquaculture Breeding and Natural Resource Conservation: A Review. Aquaculture 544).

Without wishing to be bound by theory, it is contemplated that polyploidy in kelp could yield the additional advantages sometimes found in other triploid species such as increased individual cell size (Orr-Weaver 2015) or heterozygosity and heterosis or hybrid vigour (Sattler et al. 2016).

Another route to producing sporeless varieties could be selecting wild individuals with naturally occurring recessive sporeless alleles and breeding complementary males and females so that the sporeless allele is homozygous and dominant. For example, as meiosis is a crucial step for spore production, natural mutations found on meiosis checkpoint genes could result in sporophytes that will not produce meiospores and thus be sporeless. Likely candidate genes for controlling meiosis and fertility, OUROBOROS and SAMSARA, have been identified in the model brown alga Ectocarpus, and are responsible for conversion of the sporophyte generation into a gametophyte (Arun et al. 2019). Such a cross can be achieved within one generation using gene sequencing to identify appropriate mutations on such genes. Whole genome sequencing on kelps is underway (Mao et al. 2020, Molano et al. 2022, Huang et al. 2023). A future dedicated macroalgae germplasm bank of a large diversity of species would greatly aid in screening and producing naturally sporeless varieties (Wade et al. 2020; Molano et al. 2022).

### Potential Benefits of Using Sporeless Kelp Sporophytes

The risks of domesticated farmed kelp interbreeding with the wild population include reduction in genetic diversity and adaptation, alteration of population structures and composition, and outcompeting of wild variants by domestic variants (Laikre 2010; Campbell et al. 2019). This loss of genetic variance reduces the natural gene pool from which kelp breeders can derive desirable traits from future natural variants and reduces resistance to disease and adaptability to changing environmental conditions. Therefore, it benefits the environment and the industry to minimise or eliminate as far as possible the genetic interference of cultivated variants on wild kelp populations. In the marine environment, the consequences of individual spores from a kelp farm escaping into the surroundings are currently unquantified. However, depending on the origins and size of the farmed population and the physical distance from wild populations, there could be a potentially significant impact (Valero et al. 2017; Campbell et al. 2019; Goecke et al. 2020). The likelihood for an escaped cultivated individual to encounter a wild individual and possibly reproduce is higher in nearshore environments than the open ocean, as wild kelp populations are more common nearshore, and suitable natural habitat is scarce offshore. Currently, most kelp farms are located nearshore. However, if sporeless varieties were cultivated, the chance of any escaped individual establishing itself in the surrounding environment and reproducing may be much lower or nonexistent. This could enable the cultivation of non-native and/or selectively bred species in any location, subject only to the species' tolerance of the environmental conditions, and the carrying capacity of the cultivation site, and regulatory permissibility.

Development of sporeless varieties would allow for regulatory freedom and support of selectively breeding for superior production traits whilst protecting natural populations. Planting superior strains using this approach could lead to more reliable and productive harvests which will increase the efficiency and cost-effectiveness of the farming and harvesting processes. Lower costs may make the product more attractive to companies and consumers and increase demand. Multiple native and non-native species may be grown and inter-cropped to make more diversified farms (and avoid monocropping), increase resiliency and productivity, and reduce susceptibility to disease and pests. Farmers would no longer be constrained by the seasonal production cycles of wild kelp and may be able to select for traits (e.g., temperature tolerance, resistance to biofouling) that allow for planting and harvesting all year round or at least a greater proportion of the year. Altogether, this will make upscaled seaweed farming a more financially profitable pursuit and realise socio-economic, environmental, and climate benefits. Using gametophytes of genetically desired varieties will lead to the emergence of more successful nursery or specialized “seed” companies which maintain an array of gametophytes of different genotypes to be supplied to farmers in different environments. The need for this will arise from the fact that most farmers will require new parental material after each harvest if their seaweed is sporeless and cannot be propagated for the next generation. This requirement will propel the development of specialised nurseries and test farms for the selective-breeding and propagation of superior and sporeless strains to support robust expansion of a more profitable seaweed industry.

An additional benefit could be the propagation of potentially invasive or non-native seaweed species. For instance, Undaria pinnatifida (“wakame”) is among the most promising foods for the future as it is full of balanced proteins and healthy bioactive compounds. However, it is also listed among the 50 most invasive species (South et al. 2017). French farms were established in the 1980s to farm wakame, but they were promptly closed after its invasive potential was revealed. Still, wild wakame populations remain abundant around the Mediterranean Sea, Southern California, Baja California, Mexico, and New Zealand. It is contemplated herein that developing sporeless “wakame” could enable it to be profitably farmed in a range of environments whilst eliminating its invasive potential.

In some embodiments, the use of double mutants and/or multiple sterility genes can reduce the frequency of genetic reversions to be so low that this technique is stable enough to provide superior safety for use in seaweed farms. It is possible that this method may produce more stable sporeless sporophytes than using polyploidy, or that the methods may be complementary.

Meiospore dispersal is naturally limited due to the short time that they are viable (<a day) before they settle, and is largely driven by water currents (Norton 1992; Dayton 1985; Billot et al. 2003; Bartsch et al. 2008). Meiospore dispersal distance varies and is rarely more than 1 km (Fredriksen et al. 1995; Gaylord et al. 2006).

### DISCUSSION

The use of sporeless kelp sporophytes to aid the scale-up of seaweed farms with low environmental risk is very promising and a promising and logical next step for the seaweed industry.

### REFERENCES

Aiking H, de Boer J (2020) The next protein transition. Trends in Food Science & Technology 105:515-522.
Andrus C F (1971) Production of seedless watermelons. US Department of Agriculture.
Arun A, Coelho S M, Peters A F, Bourdareau S, Pérès L, Scornet D, Strittmatter M, Lipinska A P, Yao H, Godfroy O, Montecinos G J (2019) Convergent recruitment of TALE homeodomain life cycle regulators to direct sporophyte development in land plants and brown algae. Elife, 8, p.e43101.
Augyte, S., Yarish, C., Redmond, S., and Kim, J. K. 2017. Cultivation of a morphologically distinct strain of the sugar kelp, Saccharina latissima forma angustissima, from coastal Maine, USA, with implications for ecosystem services J Appl. Phycol. 29 (4): 1967-1976. doi.org/10.1007/s10811-017-1102-x.
Augyte, S., L. Lewis, S. Lin, C.D. Neefus and C. Yarish. 2018. Speciation in the exposed intertidal: the case of Saccharina angustissima comb. nov. & stat. nov. (Laminariales, Phaeophyceae). Phycologia 57 (1): 100-112 (doi.org/10.2216/17-40.1).
Augyte, S., G. Wikfors, S. Pitchford, M. Marty-Rivera, S. Umanzor, C. Yarish, D. Bailey, & S. Lindell. 2020. The application of flow cytometry for kelp meiospore isolation. Algal Research 46, March 2020, 101810; doi.org/10.1016/j.algal.2020.101810.
Bak U G, Mols-Mortensen A, Gregersen O (2018) Production method and cost of commercial-scale offshore cultivation of kelp in the Faroe Islands using multiple partial harvesting. Algal Research 33:36-47.
Barbier M, Charrier B, Araujo R, Holdt S, Jacquemin B, Rebours C, Abreu H, Bruhn A, De Clerck O, Funderund J, Golberg A, Handa A, Ktari L, Neumann F, Peteiro C, Ronan P, Stévant P, Tamigneaux E, Timmermans K, Wichard T (2019) PEGASUS-PHYCOMORPH European Guidelines for a Sustainable Aquaculture of Seaweeds.
Barrento S, Camus C, Sousa-Pinto I, Buschmann A H (2016) Germplasm banking of the giant kelp: Our biological insurance in a changing environment. Algal Research 13:134-140.
Bartsch I, Wiencke C, Bischof K, Buchholz C M, Buck B H, Eggert A, Feuerpfeil P, Hanelt D, Jacobsen S, Karez R, Karsten U, Molis M, Roleda M Y, Schubert H, Schumann R, Valentin K, Weinberger F, Wiese J (2008) The genus Laminaria sensu lato: recent insights and developments. European Journal of Phycology 43:1-86.
Billot C, Engel C R, Rousvoal S, Kloareg B, Valero M (2003) Current patterns, habitat discontinuities and population genetic structure: the case of kelp Laminaria digitata in the English Channel. Marine Ecology Progress Series 253:111-121.
Bouvier L, Fillon F R, Lespinasse Y (1994) Oryzalin as an Efficient Agent for Chromosome Doubling of Haploid Apple Shoots in vitro. Plant Breeding 113 (4): 343-346.
Buschmann A H, Camus C, Infante J, Neori A, Israel Á, Hernández-González M C, Pereda S V, Gomez-Pinchetti J L, Golberg A, Tadmor-Shalev N, Critchley AT (2017) Seaweed production: overview of the global state of exploitation, farming and emerging research activity. European Journal of Phycology 52 (4): 391-406. Available on the World Wide Web at doi.org/10.1080/09670262.2017.1365175.
Campbell I, Macleod A, Sahlmann C, Neves L, Funderud J, Øverland M, Hughes A D, Stanley M (2019) The Environmental Risks Associated With the Development of Seaweed Farming in Europe-Prioritizing Key Knowledge Gaps. Frontiers in Marine Science 6:107.
Charrier B, Rolland E, Gupta V, Reddy C R (2015) Production of genetically and developmentally modified seaweeds: Exploiting the potential of artificial selection techniques. Frontiers in Plant Science 6:127.
Chauvin J E, Souchet C, Dantec J P, Ellissèche D (2003) Chromosome doubling of 2× Solanum species by oryzalin: method development and comparison with spontaneous chromosome doubling in vitro. Plant Cell, Tissue and Organ Culture 73 (1): 65-73.
Dayton P K (1985) Ecology of Kelp Communities. Ann. Rev. Ecol. Syst. (16) 215-45.
Ebbing A P, Pierik R, Fivash G S, van de Loosdrecht N C, Bouma T J, Kromkamp J C, Timmermans K (2021) The role of seasonality in reproduction of multiannual delayed gametophytes of Saccharina latissima. Journal of Phycology 57 (5): 1580-1589. FAO (2020) The State of World Fisheries and Aquaculture.
Fredriksen S, Sjotun K, Lein T E, Rueness J (1995) Spore dispersal in Laminaria hyperborea (Laminariales, Phaeophyceae). Sarsia 80, 47-53. doi.org/10.1080/00364827.1995.10413579
Gaylord B, Reed D C, Raimondi P T, Washburn L (2006) Macro-algal spore dispersal in coastal environments: mechanistic insights revealed by theory and experiment. Ecological Mono-graphs 76:481-502.
Goecke F, Klemetsdal G, Ergon Å (2020) Cultivar Development of Kelps for Commercial Cultivation-Past Lessons and Future Prospects. Frontiers in Marine Science 8:110.
Grebe G S, Byron C J, Gelais A St, Kotowicz D M, Olson T K (2019) An ecosystem approach to kelp aquaculture in the Americas and Europe. Aquaculture Reports 15:100215.
Guo X, DeBrosse G A, Allen Jr S K (1996) All-triploid Pacific oysters (Crassostrea gigas Thunberg) produced by mating tetraploids and diploids. Aquaculture 142 (3-4): 149-161.
Huang M, Robbins K R, Li Y, Umanzor S, Bailey D, Aydlett M, Schmutz J, Grimwood J, Yarish C, Lindell S, Jannink J (2023) Genomic selection in algae with biphasic lifecycles: A Saccharina latissima (sugar kelp) case study. Frontiers in Marine Science 10. Available on the World Wide Web at doi.org/10.3389/fmars.2023.1040979
Hu Z M, Shan T F, Zhang J, Zhang Q S, Critchley A T, Choi H G, Yotsukura N, Liu F L, Duan D L (2021) Kelp aquaculture in China: a retrospective and future prospects. Reviews in Aquaculture 13 (3): 1324-1351.
Hwang E K, Boo G H, Graf L, Yarish C, Yoon H S, Kim J K (2022) Kelps in Korea: from population structure to aquaculture to potential carbon sequestration. Algae 37 (2): 85-103.
Jouaux A, Heude-Berthelin C, Sourdaine P, Mathieu M, Kellner K (2010) Gametogenic stages in triploid oysters Crassostrea gigas: Irregular locking of gonial proliferation and subsequent reproductive effort. Journal of Experimental Marine Biology and Ecology 395 (1-2): 162-170.
Kim J. K., C. Yarish, E. K. Hwang, M. S. Park and Y. D. Kim. 2017. Seaweed aquaculture: cultivation technologies, challenges and its ecosystem services. Algae 32 (1): 1-13.
Kim J K, Stekoll M, Yarish C (2019) Opportunities, challenges and future directions of open-water seaweed aquaculture in the United States, Phycologia 58:5: 446-46. DOI: 10.1080/00318884.2019.1625611
Laikre L (2010) Genetic diversity is overlooked in international conservation policy implementation. Conserv Genet. 11:349-354.
Li X, Zhang Z, Qu S, Liang G, Sun J, Zhao N, Cui C, Cao Z, Li Y, Pan J, Yu S (2016) Improving seedless kelp (Saccharina japonica) during its domestication by hybridizing gametophytes and seedling-raising from sporophytes. Scientific Reports 6 (1): 21255.
Li Y, Umanzor S, Ng C, Huang M, Marty-Rivera M, Bailey D, Aydlett M, Jannink J L, Lindell S, Yarish C (2022) Skinny kelp (Saccharina angustissima) provides valuable genetics for the biomass improvement of farmed sugar kelp (Saccharina latissima). Journal of Applied Phycology 34 (5): 2551-2563.
Liu X, Bogaert K, Engelen A H, Leliaert F, Roleda M Y, De Clerck O (2017) Seaweed reproductive biology: Environmental and genetic controls. Botanica Marina 60 (2): 89-108.
Loureiro R, Gachon C M M, Rebours C (2015) Seaweed cultivation: Potential and challenges of crop domestication at an unprecedented pace. New Phytologist 206 (2): 489-492.
Lüning K and Neushul M (1978) Light and temperature demands for growth and reproduction of laminarian gametophytes in southern and central California. Marine Biology 45:4, 297-309.
Mao X, Augyte S, Huang M, Hare M P, Bailey D, Umanzor S, Marty-Rivera M, Robbins K R, Yarish C, Lindell S, Jannink J L (2020) Population Genetics of Sugar Kelp Throughout the Northeastern United States Using Genome-Wide Markers. Frontiers in Marine Science 7:694.
Mather C and Fanning L (2019) Social licence and aquaculture: Towards a research agenda. Marine Policy 99:275-282.
Molano G, Diesel J, Montecinos G J, Alberto F, Nuzhdin S V (2022) Sporophyte Stage Genes Exhibit Stronger Selection Than Gametophyte Stage Genes in Haplodiplontic Giant Kelp. Frontiers in Marine Science, p.1766.
Norton T A (1992) Dispersal by macroalgae. Br. J. Phycol. 27:293-301.
Oppliger L V, Dassow P, von Bouchemousse S, Robuchon M, Valero M, Correa J A, Mauger S, Destombe C (2014) Alteration of Sexual Reproduction and Genetic Diversity in the Kelp Species Laminaria digitata at the Southern Limit of Its Range. PLOS ONE 9 (7): e102518.
Orr-Weaver TL (2015) When bigger is better: The role of polyploidy in organogenesis. Trends in Genetics 31 (6): 307-315.
Patyshakuliyeva A, Falkoski D L, Wiebenga A, Timmermans K, de Vries R P (2019) Macroalgae Derived Fungi Have High Abilities to Degrade Algal Polymers. Microorganisms 2020, 8:52.
Redmond S, Green L, Yarish C, Kim J K, Neefus C. 2014. “New England Seaweed Culture Handbook” Seaweed Cultivation. Paper 1. Available on the World Wide Web at opencommons.uconn.edu/seagrant_weedcult/1/Sattler M C, Carvalho C R, Clarindo W R (2016) The polyploidy and its key role in plant breeding. Planta. 243:243-281.
South P M, Floerl O, Forrest B M, Thomsen M S (2017) A review of three decades of research on the invasive kelp Undaria pinnatifida in Australasia: An assessment of its success, impacts and status as one of the world's worst invaders. Marine Environmental Research 131:243-257.
Sree Ramulu K, Verhoeven H A, Dijkhuis P (1991) Mitotic blocking, micronucleation, and chromosome doubling by oryzalin, amiprophos-methyl, and colchicine in potato. Protoplasma 160:65-71. State of Alaska (2022) Aquatic Stock Acquisition and Transport Permit (5 AAC 41.290). Available on the World Wide Web at adfg.alaska.gov/index.cfm?adfg=aquaticfarming.acquistions
Torres M D, Kraan S, Dominguez H (2019) Seaweed biorefinery. Reviews in Environmental Science and Bio/Technology 18:335-388.
Umanzor S, Li Y, Bailey D, Augyte S, Huang M, Marty-Rivera M, Jannink J L, Yarish C, Lindell S (2021) Comparative analysis of morphometric traits of farmed sugar kelp and skinny kelp, Saccharina spp., strains from the Northwest Atlantic. Journal of the World Aquaculture Society 52 (5): 1059-1068. UN (2017) World Population Prospects: The 2017 revision. Key findings and advance tables.
Valero M, Guillemin M L, Destombe C, Jacquemin B, Gachon C M M, Badis Y, Buschmann A H, Camus C, Faugeron S (2017) Perspectives on domestication research for sustainable seaweed aquaculture. Perspect. Phycology. (4): 33-46.
Visch W, Rad-Menéndez C, Nylund GM, Pavia H, Ryan M J, Day J (2019) Underpinning the development of seaweed biotechnology: cryopreservation of brown algae (Saccharina latissima) gametophytes. Biopreservation and biobanking 17 (5): 378-386.
Visch W, Kononets M, Hall P O J, Nylund G M, Pavia H (2020) Environmental impact of kelp (Saccharina latissima) aquaculture. Marine Pollution Bulletin 155:110962.
Wade R, Augyte S, Harden M, Nuzhdin S, Yarish C, Alberto F (2020) Macroalgal germplasm banking for conservation, food security, and industry. PLOS biology 18 (2): 3000641.
Wadsworth P, Wilson A E, Walton W C (2019) A meta-analysis of growth rate in diploid and triploid oysters. Aquaculture 499:9-16.
Wang X, Cheng Z M, Zhi S (2016) Breeding Triploid Plants: A Review. Czech J. Genet. Plant Breed. 52 (2): 41-54.
van den Burg S, Selnes T, Alves L, Giesbers E, Daniel A (2021). Prospects for upgrading by the European kelp sector. Journal of Applied Phycology 33 (1): 557-566.
Van Zanten H H E, Van Ittersum M K, De Boer I J M (2019) The role of farm animals in a circular food system. Global Food Security 21:18-22.
Yang, H., Huo Y, Yee J C, Yarish C. 2021. Germplasm Cryopreservation of Macroalgae for Aquaculture Breeding and Natural Resource Conservation: A Review. Aquaculture 544, available on the World Wide Web at doi.org/10.1016/j.aquaculture.2021.737037.

1. An algal gametophyte comprising one or more mutations of Spo11, Mre11, Rad50, HEMI, SAMSARA, OUROBOROS, and Tables 3-7.

2. A combination of algal gametophytes comprising:
a first algal gametophyte comprising at least a first mutation selected from:
a mutation of Spo11, Mre11, Rad50, HFM1, SAMSARA, OUROBOROS, and Tables 3-7; and
a second algal gametophyte comprising at least a second mutation selected from:
a mutation of Spo11, Mre11, Rad50, HEMI, SAMSARA, OUROBOROS, and/or Tables 3-7.

3. An algal sporophyte comprising:
at least a first mutation selected from:
a mutation of Spo11, Mre11, Rad50, HEMI, SAMSARA, OUROBOROS, and/or Tables 3-7; and
at least a second mutation selected from:
a mutation of Spo11, Mre11, Rad50, HFM1, SAMSARA, OUROBOROS, and/or Tables 3-7.

4. The algal sporophyte of claim 3, wherein the algal sporophyte is infertile or sterile.

5. The algal gametophyte of claim 1, wherein the at least a first mutation and the at least a second mutation are not the same mutation or are not in the same gene.

6. (canceled)

7. The algal gametophyte of claim 1, wherein the mutation, the at least a first mutation, or the at least a second mutation comprises a mutation in a gene selected from the group consisting of: Spo11, Mre11, Rad50, HFM1, SAMSARA, and OUROBOROS.

8. (canceled)

9. The algal gametophyte of claim 2, wherein the at least a first mutation and the at least a second mutation each comprise a mutation in a gene selected from the group consisting of: Spo11, Mre11, Rad50, HFM1, SAMSARA, and OUROBOROS.

10. The algal gametophyte of claim 2, wherein the at least a first mutation and the at least a second mutation each comprise a mutation in a gene selected from the group consisting of: Spo11, Mre11, HFM1, and Rad50.

11. The algal gametophyte of claim 2, wherein the at least a first mutation and the at least a second mutation collectively comprise a mutation in each of: Rad50 and Spo11.

12. The algal gametophyte of claim 2, wherein the at least a first mutation and the at least a second mutation each comprise a different mutation in Rad50.

13. The algal gametophyte of claim 1, wherein the mutation, the at least a first mutation, or the at least a second mutation comprises a loss-of-function mutation, a nonsense mutation, or a missense mutation.

14-19. (canceled)

20. The algal gametophyte of claim 1, wherein the algal gametophyte or algal sporophyte is a brown alga (Phaeophyceae class), Laminariales, sugar kelp (Saccharina latissima), Saccharina latissima forma angustissima, or giant kelp (Macrocystis pyrifera).

21-24. (canceled)

25. The algal gametophyte of claim 1, wherein the algal gametophyte is a male gametophyte or a female gametophyte.

26. (canceled)

27. A population of infertile, sterile, or non-reproductive algal sporophytes.

28. (canceled)

29. (canceled)

30. A method of cultivating or producing algae or algal material, comprising:
a. contacting an isolated male gametophyte of claim 1 with an isolated female gametophyte of claim 1; and
b. cultivating or growing the resulting sporophyte.

31. A method of cultivating or producing algae or algal material, comprising:
a. cultivating or growing a sporophyte resulting from an isolated male gametophyte of claim 1 with an isolated female gametophyte of claim 1.

32. A method of providing, cultivating, or producing a sterile, infertile, or non-reproductive algal sporophyte, comprising:
a. contacting an isolated male gametophyte of claim 1 with an isolated female gametophyte of claim 1.

33. A method of cultivating or producing alge algae or algal material, comprising:
a. cultivating or growing a population of sporophyte resulting from a population of male gametophytes consisting of one or more isolated male gametophytes of claim 1 with a population of female gametophytes consisting of one or more isolated female gametophytes of claim 1.

34. A composition of algal gametophytes comprising one or more mutations.

35.-38. (canceled)

39. A plant that is unable to reproduce, wherein the plant comprises one or more mutations or mutated gene products that result in infertility.

40-42. (canceled)


SEQUENCE LISTING (10 sequences)

SEQ ID NO: 1 - DNA, 4140 bp, Macrocystis pyrifera
  source 1..4140: mol_type = genomic DNA
atgctcctccgaacacgtctcgtcgcagagagcgacaagggggggtctacgatcgaagtcgagatagcgaccctcgagcgcctgaatcaggaggacacaaggctctgcgacgatctgcgcgcgtcatgccgcatagggaacaacacccgcggggggggcgtgcagcagggcaagctctcctgctgggccctggtccaaggcggtggggtttccaaaagggcgttttcgtcgtcgactgctaaagcactcatcgatggggccgtttccggtagacgccagagcaccggaacagggagcggcgacgacgacagcgggggaagcaatccagggtctcggaaacgtgttttctcaaagtacgatgaggagacgacgagcatcttgacggaatggtttctctcgcacaagaggtggccctacccagctgccaaggagaaggatgccctggctgaggctaccaacctcaccaccttgcagatttcaaactggttcaccaacaagcggaaaaggcactggactccggtcatcaagaagcgcacgaggtcaccccgggacttcttcgagttctgcgtcatgaacccgaaagaccacgcgctaaacaacgtgcggccgcacagccaccagcccccccagcgggctggcgtagctgtgggcgcggacagcaacccttacacccttgcggccatggcgcagcggggcggcatcaacggccttcatgtgccggtggtgggcgacatgcgccagatggcgggagcggtccatcctggagccgcgcggggctttacgacgccaggtcatagtggcctgcccggcataagcttcgacggcctgcccactgcccccgcggatgcagcacacaacagcgttgttatgccggggggggcgggcggcggcggagggtgggttcgcacagccgccgggatggagaagaagggcgtcgacaacgccgttgcatggctggaccattcgcggcagcagcagcagcagcaggtacagcagagtcagcagcagcagccgcagcagcagcagcaacaaggccagcaccctggctcgcttgctcccggcctgatgtacgcttccaaggccgccctgcatgccgctaacatggaaaactccaccccccatcaccccgtgcctctgtggctcgcgccgcccacgggcatggaccagcggaggccgcaccccatgggttatcctgacgccaaccacagaccctctcctgccggagcttcttccatcgcgatacctcagcacgcggacgagcacagggatgcggcgcgaatgaggctgcacgccccgcagtacgccgatgtccgcgggtttgatgcaagcaaatcacagcagaggtttatcgaacatcccgcctcgttttactccaaaggaccggccggtcccgacgtgatgcactatcccgccgcaccttccaacgccaacacgggaggcgcgatttccggcggcggcggcggcgacgagtacgcttggcgcgatgctgccgtgaggcctgagacttttcctcccattgctgcgttcgccgctagcgggggcaaaaccaccagcaacgaaaagtgtgaccatgtcactgcctatgccagcaccggggaccaaaacccactgccagagagaaagggatcacgagggtatgggcgtcttgccagctctggcgacaggctggaggcgggtggagagggcatcagcttgcaggagacgcaggcgaggatccaggaactggagaggagcgagttcgatctcaagatgaggctgttttacgccgaggaacagcttgagaacgctgccgggcgagcggatgcggtacaacttcatcgggaggtcgcagacgcaaagcggcggagagcccacgccgaggaccgcacacgagtggcgggcgaggcccttgccggtttggaggccgactgcaggaagcttcaagctcaggtgcgagatgtggaagcgaggaggaagaaggaccaggagacaagggccgaggtcgaagactcagccacgttcaggctaaagaaacacataatgcttctcgaggataccctccgtgcagagcgtgtcgagaacgctcgccttcgtggcgccagcaccggctgccgaggcagtgacgacgacctacccggggaggctggactgagacgctccagaatacgccgaagaagcactggagacgacgggcttggaaacccgaacccctggagttctgcccgtggcaacgtcggcggctgttttgagcccaagcagcgagagcaagagaggcagctggtggccaaggtcggggcgcaagattcgtcaatgcggttgctccgcgccgacgtaaccaccttgaggaaacgcttacggagacagggggaggtcctggcgaaacagatagaggagaacgaggtggtgcgccattcggcagaacaggtggcctgtgttgaggcggaggaaatctcgaggctaacggaggcactcgagcaagctgtagcggcagccagtcgagagaccgcgaagcgacggacggcggaaaaacgactggcggcgctagatacacgcctgatagcgttgaaaaccaacggcacacgcacgccgcgatcggtctcccggacaccgtccccccactctaacacgcgaagcaatgacgggtctacgatggatacaggatgggcggctcacaggaagcctaggtctcccgccatgggtgcctcctccccctcttcagcgtggacgggtagcgggagccgcgagccgaaccgcgggagggaaaccaagtcgctcccgccgcccaagaggggggccaccggcagcactgccgcctccagggggcggcggcttgcccggaacagcacaggtgcgcgccgggaggggggtcgcgtttgcgaaaaaggacggtcctcatcttcccggcgggggagtcgagactccgagaaggaacattggaggccgccaggatgttctcctccgccaagccctgttggtcgacggggcgggcgaagggccagcttctcgggcggggcgggcgctgctcatggccgccaccacccccgcggccgtggacgggcaggagaggtcgggcccgcccctgaccttaaaacctcctcgcttgctctttcgaacgaatcggtcaaaactggcgctagcggcggtggtggcggtggacgagggcgagaagctgtatctccttttgatacagggcggttgggggcgagcgagagatgtgccagcccgctgcgaaacggagaggggccgtcggatcgctcctccgtagggaggcgcaagcgtggggattggaaggggggtgttgacggtggcttgggaggctcttgccggcccagggtgagcggcggtggtagctgcggcgtcgcaggcgcaggcaggttatcccgcaattctgtggcgagccgcgcctctacttccacggcgggggcgactgaggaagatgtacacaaacatgttgctgctcgtggtacgagttctgggctgccaccagaaagacaatcagacgttgccctgaaagggcagggcagcggtgccggtgacgcgcgtgtaataagcacgcctgccgacgatgtttggtccggcaggatgtcggaggtgggtgccatggcggccgctatccgcggcgtggatttgtccgaaaccgtcgcccgggagctccgcttggccgtggctaccaccgccgcccccgctaccgctgccgcactgcctggttaccgcgctactcgcggcgcgaggcgggggatcgcggagggcagggcgaggaaaagcacgccggcttacggtgccggtaacgaaggcgagaagacttccccgcgaagcgattgcggggacgagcggcggaacggcgcgcccgagtcctgggagactagtgaggcgcggagccggcaccggcgcggcggaagcgagtacacgagtgggggtttcaagatgaatggcagagtcttaggggggtcgccgggggatggggggcgggcgctcaagagcagagaagttgtggaactagagagtgatgttcagcacatattccagttttttgaggcacgggatcgagtggatacagttgcccgggaacgctcggggttggccgggggaagaggaggaggggatagtgaggagggagaggacgaggattttttctccgccatggctggggcacttgcttga

SEQ ID NO: 2 - DNA, 1107 bp, Macrocystis pyrifera
  source 1..1107: mol_type = genomic DNA
atggacgtagacgtgccaccgggatcaagagccgccttgggagccctgcccccggggaacacgatggtgccgactggcgatcgcggcactggtgttgacatccgacctgctgattcggcaccgactctcgcgaaaggactcctgccagctgcccctccagcggcaacacaaacaggcgaaatcatggtgcctcccggcccctccacttcggactcgatggccaccaaggatcagcttgtccaggcccaggagtacctgggaacgctccgctcggaccgcgcggaatcgtgccgggagctggaggaggagaggaaggtttttctggactccccctccacccctgagcaggaacggcgcttccgcctcatctaccagcttctgcgataccaggtccacacccacacccacagcatgtattctatcctgcaggagaagctggcggcgatggcgagggtacaggctatggttggcggcggcgaggacgacccggatgtcaaagccctcacaatcgcactagtcaacgggagaattgtcgacgcaacgatcagggtggtagagcgcgccctcgaggcgatgcgcgagcttaacggcacgggcgcggtgatgaaggaaccttgtccgccgtccgcctgggagcaatcgaagaaggagcaagagcccactctgccctgcgtgcccactgacccgtctgggcagaagccggccaagcgatcccgcttgcccgcggcggcagtgaaagagatgaggacgtggcttgaccagcactgggacgaccctgtgcccacccacgaggaaaaggccggctttgctcaaaggcacgacatcacaaccaagcaggtcgaaaactggttcatcaacatccgcatgagagagtggcggcccgccatgcgccgggccctggatcacgccgaggagaccggctcgtatgcggaattctcgaggctcctccagcacaccgcagacggcaacgtcttcaagaagttcctcgaggagcgcgagatgggccacggctccgccttggtgcccccacccagcctccagcgcttcatcggtgcggaaggcttcgcagcattcgtgggagaaaggtccaacaagcgcatgcgctga

SEQ ID NO: 3 - DNA, 834 bp, Macrocystis pyrifera
  source 1..834: mol_type = genomic DNA
atgatcgtcggggcaaatgggtgcggcaagacgaccgtgatcgaatgcctcaagtacgcctgcaccggggcgctcccgcccggcgcgcgcaacggccattccttcgtccacgacccaaaggtgtcgggcacgaacgaggtgaaggccagcatcaagctccgcttcgccggccgggacggccgagtgagcgtggtcatccggtcgctccagctcactcagaagcgggtgtctctcgagtttaaggcgctcgacggcgtaatcaggacgaaggatgacaacggtgtgagcgtgagcatcaaccacaagtgcacggagatggacaagcacgtggatctgctgcagggaaagaagcgatccctcgagctaacgtgccgagaccttggcgtgcgcatggcagagaagaaagagaacatcggcgatgctctaagcacggagtcggacgaggccctcgacaggaacctcagggagttcgacgccaggctggacacgtggtcggtcagacggtacacgtcagaggacatagcgaccttcgaggaggcgctccgcggcgcccacgacaaggcccaggcggtgctcaacgagactcgggccagcactcggctggaggacgagtcgatgaacgcggagatgaacactcttaagctggagcagaggagcagctcggccgccgtgcacgcaaaggagcgcgagctgcagaagattcagggtgaagccatgtcgttggcgcagggcggagggtctgtgggtgccgggagccaaagccagctgggtacggcgacagtgtccaagctggagcgggagctgaaggctgcggagaaggctaagaagcagtga

SEQ ID NO: 4 - DNA, 1179 bp, Macrocystis pyrifera
  source 1..1179: mol_type = genomic DNA
atgccggtgaccgtgctctcttcgcccctggacgtgcgattgcatgaccgagaacgcgttctcgacaagatccaagcgatcgtggtagactggctggaccagctggccggcggggatctcccagtcttcgaagcagcggacgaggatcaagaagacgatgatatcgtgggggaggaagctgacaacctccgagagggggcaacttttcgggcacgcatcctctgcgtattgagcttctcaacgagactcctggagactcgtaaaactgccacgcaaagagagatttactacacctacgtcaagcactttgcgaaccagaccaaatgcaacgaggccatactggactgctgcgcgttgctgggtgaaccccggcattgcctcgggatagtggcaagcccaagggggtaccttgcagggcgcatctctcttcgggggcagggattcgaagactggcaagatcccaggtcgacgggaggaacatcaggcctcgccatcacggcagactggctgactcgccccctcgagttgaagtccgacgcccgctacgtggtggtcgtcgagaaggatggcgtgttcaatcgcatggtggaggacggatttttcgacaggcaagcatatacaaaaacacaccccctcggtcgattgataccttcgattctcgtgaccggtcagggctttcctccgctggccgtacgcgcctgcgttcacaggctggctgaggccttcgatctgcccgttcttggcctcgtcgattgcaacccatttggactgagcgtttttcttctgtacaaggaaggctcgtctcggcagggccggggttctgtgaagtatgcagttccaggaatgaagtgggtaggcctccgcccgtcccagctcgaagacttggacctcccccaccaggtacaccaggaactttcgtctctggatattgccagggcgcggtcgatcgcaaatctgccaatcgtttccgagaaccccgacacatacgggcaggaggtcgcgtactgggccgacgaaaacaacccaggcaagcaagtcaacaggtccgcgtgcccagtcggtgactcgatggccgctactgcccgagcttcaacatatctgcgattcgtggttgaacaggcaacgatcatgtgttttagtggcgggcgatactcgtctcgttggcaggaaaaaatctag

SEQ ID NO: 5 - DNA, 7154 bp, Macrocystis pyrifera
  source 1..7154: mol_type = genomic DNA
atgttgtttttttaccgcttccgccaagtgtcgttaccgccgaaaaatgaaaaaccgccaaccgccaaccgccaaaaaaagacgccgtacgtattgctttaccgccgaaaatataccaccatattttggttttccgcttccgccaaagtcgttaacgccgaaaacgagaaaaccgcaaaccacctaaaaattaccaccgtatggaaaaataccgccccaactgtgtccgctcgaaaaaccgttacagacgaccacgctggacgaaaaggataccgcccagcagtcacgaccatcacgacgtcgcagccgcgattgagttcggtactgcggttgaccaatgctccaaacgcgcgtgctctgttcgaccttgacgctgagatactcacgagagagagagctgaagcgcaatggaagagccaagctgacgcgaaatattcggcgcccttcacatggaaggcgttttccgagaaggtccccgcgagcgaggcgcagagagctacggtggagcaagacaagtttcgcaggtactctagactggcggggaagctagcgggaggcgacgacggggcggcgtttcccaacgcggaccgggcgctcttccaggcgatcaactcggcgaagggtgcggcggaacaggccaagagggaccttgagctcgtggtgggaaaagtgcctgccaaagactgggccgaggccacatccctcgccaaggagctccacgcgtggaggcgtgaacgcgtgcggggaccgccacaacgacaaccgtcgcagcagccccagctcaaggcccagccgaggccgcgcgcaggaacgccacccaggggagggggggggggaggcgacctcctgtttggcagtggtatcgtgttcaacttcgaccttgactgtccggaggaggaggatgaccttgaccgagctttccctggggggctcgtacctggccgctctgttggtgttggtggtagtggtggtggcgatcgagaaggtcgaagcgggggtttcgctgcggaaggaggcgttacgggcctagggtttccggggcgggctcctggcctggagggggtagggggtggtagcgcggcggaggtggcggaggggggggtggaggcgagggctgtcgatgcccacgaaccttggaagggagaggagaggggggaggggggaggggcgggggggtggaaggggggcgcggaggaggagaagggggagtggaaagatgcatcacgccccgctctgccgcctgccttgggtggaaagaagggggctgtggcggccgaggatgagtttgatgccggctggctgtactccaggcagccgcgcgataggaagagcgttgtcgcggaagcaaagcagtgtagcaaagggactacaacgccgcacacgcggggcttcccggaggcgtacgatgtgcgggtcgagggggcgctgccgtcgggctctcgggccttcgcttcctttactccgccctttgcctttactattgaagtgttgttggggtccctgttcgacctcctcggggaggcgggcctggagctgatgatggacctcgtagccaagacggcgtcgctcagacggatcgatgctcgcgacctgtttgccatagccgacgcccatggtggtggtggtggtggtggcggcgcagggggcgtagggggtggtgccgggggcgcggcgggtatggggggtgtgggagggttcgggggagcgccggaggaggcgcaaggaagatcggggccgagcatctcccatcaaatcaagatttggtccacttcggagaaagaagcggagaaggctcgccggaaaaatctcaagaaggccgcgaagcgggggcagagggggcccgggggggaggaaggcggttcggcggcggggggcgcagggttggactggctgcagtccgtgggattcgaggacgaatacctgaaacaggagcgcctgctgggtttgcaaggaggcgggacgcgagcgggaggaggggacaacgaggaaatgatgctggcgtcccttgctccggaggggacgcgggagtggcatgacaggcgctcggggctgcctgcgggagcgacgaaaacagtggtggccggccagtatgagcaggtccacatcccccctccgaaactgccgggccgaaaagacggggatgacctggtgcccatcactgagctcgagcagtgggctcagctggccttcaaggggaccaagcggctcaaccctatgcagagcaaggcgagccaactgcgcggctcgaggcttgcggctcgcgtataccatgccgcgttcagaacttcggaaaaccttctcgtgtgcgccccaacaggagcgggaaagacgaacgtggctatgctctcgcttctccagctggtgaggcagcacatccacgggggtgttctggagcggagcggtatcaaggctatctacgtggccccgatgaaggcgctggctcaggaggttgtcgccaagttctcccagagactgaagccgctgggattggtggtacgggagtacaccggtgacatgcagctgtccaagcaagaggtggaagggtcgcaggtgattgtgacgactcccgagaagtgggacgtggtgacccgcaaggggggcgacggttcgctcgtttcctccgtcgggctgatcatgatcgacgaggtgcacctccttgccgacgatcgcggtgctgtcatcgagagtatcgtggccaggacgcagcggtacatggagaccacgcagacgctcatccggctagtcgggctgtcggctacgctgcccaattaccaggacgttgcgtccttcctcagggtcaacccgagcaaagggctgttttacttcgggccggagcacagaccagtcccgctggagcagactttcatcggggttaccgagaagcagagggttcggcagcaggcgatgatgaaccgaatcgcctatcagagagcgagggaatctcttcagcgcggtcatcaggtcatggtgttcgtgcatgcgcggaaggataccgtcagaacggctgaggctattttggagctagctcagcgggacaacgccttcgatgaattttcctgcacgggctcggagcactgggggcggcatgcccgccaggttgaaaaatctcgcaacaaggagctgcgggacttgtttcaggcggggctgggctgccaccacgccgggatgctgcggtccgaccgggggctgacggagcgatcgttcgaagatggagccatcaaggttctcgtgtgcacggctactctggcgtggggcgtcaacttgccggcgcacacggtgatcatcaagggcacggaggtgtacaacccggagaagggaggcttgcaggacatcagcatgctcgacgtgctgcaggtgttcgggcgagccggacgcccccagtacgacacgagcggggaggccatcatgatcaccacgcacaagtcgcttgacaaatacctggccctgctggccaagcaaacgccgatcgagagctccttcatcaaggccttgcccgatcatctcaatgcggaagtgtcttcgggaactgtcaccactgtcgacgagggcgtgacgtggctgtcctacacgtaccttcatgtcagaatgcggcggaacccgatggcgtacggcgtgcctctgttggaccgcgaggcagaccctcagctgcttgagaggcggaggcagctcatcacccaggcggcagaaacgctagacgaccacaagatgttgcggttcgaccgccgatcgggtaatctggcggttacggacttgggacgcgccgcctcccatttctacatcagccacgagagcgtgttccgcttcaacggcgcgatgatgccaactctctccgatgccgcggccctcaacacggtctgccttgcgagcgagttcgaccaggtcaaggttcgtcccgaggagctgaaggacatggacaacatgcggaaacgctgccccctcgaggtgaaggctccgctggaggagtctgccgggaaggtcaacgtgctcctgcagtcctacatcggcggggggaggcccctcagcttcacgcttgcgtcggacaccaactatgtcgctcaaaacgccggacgcgtgtcccgtgcgatattcgagatcgccctgcgtaaggggtggtgcggacttgcgctcaccatgctggagttgagcaaggccatcgaccgccgtgtttggtggtttcagtcaccgctccgacagttcggcgtgctcccgggcacggtcctcatgaacctcgaaggcaagggggggggcggatcggaagggatcgggaggctgctcgacatggatgccggggaggtcggggcgctttgccacaaccaccgcatgggggacacggtgctgcggctggcaagatctttaccggctctgcacatcgaaacggccgtgcagccggttactcgcggcatcctcaggctgacactcagggttcacgcaagcttcaaatggcaggacaagttcaacggttccacggagtcgttttggatctgggtggaggacggggaaaacgagcacgtctatcactccgagaccttcctcctgaagaaaaaaaaaaggaacgaagcgcaagaactctccttcaacatcccggtcttcgagcccctccccgcccagtactgggtgaggtggtgcagcgaccgctgggtgggctgcgaggatgtgcagcccgtgtccttccagcaccttgtcttgccggagaggtaccccgcacacacaaccctcctggacctgcgtcctttgcccatcacagcgctgcagaaccccaagtttgagagcttgtaccggtacgaacacttcaaccctatccagacgcagctgttccatgtgctctaccacagcgacgaaagcgtcttgttgggggcgccgacagggtccgggaagactgccgtcgcggagatcgccatcatgcgcatgctcaacgagcacccgggggcgaaggcggtgtacgtggcacctctcaaggccctcgcccgagagcgcctcaaagattggcgggagaagttcgagaagaagatgggcatgggcgttctggagctcaccggcgacaataccccggacgtagacgcgctcaagcggtcgactatcatcgtcaccacccccgagaagtgggatggcgttacgaggagctggaagactcgggactacgtcaaggacaccggcttagtgatcatggatgagatccacttgctcggggaggaccgagggccagtcctcgaggtgatcgtcagtcggatgcggtacatcgcggcgtccgccgctcgcggcaacggctcctccggccaacgacaggtgcggttcgtgggcttgagcacggcgctcgccaacccgagggacctgtcggactggctcggagtgaaggacaccggcctctacaacttcaggcctagcgtccggcccatcccctgcgaagttcacatccagggataccccggcaagcattactgcccgcgcatggcgagcatgaacaagcctacgtacgcggccattttggagcacagcccggagaaaccggtgctggtgttcgtcgcttctcggcggcagacgcggctgacagccttggacctgatcagcctttgcgcaagggcggacaacccgaggcggttcgtgcggatgccggaggatgaggcgcagaacgccgcggattctgtcagagatcaagcgctgcagcacactctcgcgttcggaatcggcatccaccacgccggtttggccgagggtgaccggagcctcgtggaggcgttgtttgaacaggggaagattcaggtactggtgtgcacgtctaccctagcgtggggcgtgaacttccccgcccacctcgtcgtggtgaagggaaccgagtttttcgacggcaagagtcaacgatacgtcgacttccccatcacggatctcttgcagatgattgggcgcgccggacgtccccagttcgacgaccacgccgtcgcctgcatcctcgtccacgagcccaagaaaaacttcttcaagaagtttctgtacgagcccttcccggtggagtctaagctgcccgggtcgctgcacaaccacttgagcgccgagtgcgcgggaggggcgatcaagtcccgcaaggatgcggtggactacctgacgtggaccttctactttgtgaggctgctggcgaacccgtccttctatggtctgacggagacgtccacggatggcgttcaggaggtgcgggctaccccgttggccatggtggcgagccggtactacctggactaccgcaccatgaagctgttccagggatgcttcggcgggaaaggaggggatggggctactttggaagacctctgtcgcctgctcgctgatgcccaggagtatgctgagctccccgtgcgacacaacgaagacgttctgaacggagaactttccaagaagcttccgtgggctgtggaaaccgaggagctcgacagcccgcacgccaaggctcaccttctgctgcaggcccacttcgaccgctgcactctgccgatatcggactacgtcacagacacgaggagcgtgttggatcaggccgtgcgcgtgctaaacgccatgctagacatcgccgctgggtttgggttgctggagaccaccctggggttgctgcggctgcaccagatgttggtccaggagttggtgcaggcgggtgactgcgaagctaccgtgcgcgtttcggtggccgcgccgcacggccggagaggctcgctctggtctccgcgattcccgagggctcagaacgtgggctggtggctggtgctgggcacggaagaaggggagctgctggctctcaagaggggtacaacaacagcagcagcggtagtccacgaattaaaaggcaaacgttcgacccccgtcgtgatacgtagtcgtgataggcgtggggcgcaaacggtgccggcaaaaaaatgcggccagggtcacagcacctcctgcaaccatggcagcatcagaatcgccgccagtcacaacgtcaggcatgacgagcgaacaccaaccgcacggcgcaaaggacataatttcaacgcacacaacgtctaccgtgaaaacaaaaatgccgacataaggcaacgcccgggaccaccacggagggacagcggcggaaacgttaaactaaagcatactggtaaaagtggtaataagtggttgatcgttatgctacgtga

SEQ ID NO: 6 - DNA, 648 bp, Macrocystis pyrifera
  source 1..648: mol_type = genomic DNA
atgtccgagaatgaaagcctcagtatcttggtggcaacggatagccatctggggtacatggagcgagaccccgtgagaggcaaggactcgttcgctgctttcgaggagatgctcctcatcgccaagaagaagaaggtggacttcgttctcctcggaggagacctgtttcacgagaacaagccctcccggcgcaccctgtaccgcacgatggacatactgcgtcgccactgcatgagcgacgaccctgtcgggttccagatcatcagcgagcagtcgcagaacttcaaggacaggttcgggcacgtgaactacgaggacccttacttctctgtggggctgcctgtcttcagcatccatggcaaccatgacgatcctacccgggagggaggggtggaggcgctcgcggcgcttgacctgctgcacgtggcaaacctcgttaactatttcggcaagagtgacaaggtagatgatgtggaagtgaaccccatcttgatacaaaaggggctgacgaagctagctctttacggaatgggcagcatgagggacgagaggctcaaccgaatgtggcagcagaagaaggtgaggtttctccgccccctggaggatgacgggggcaaggacttcttcaacgtctttgtgattcaccag

SEQ ID NO: 7 - DNA, 24 bp, synthetic construct
  source 1..24: mol_type = other DNA
atgctactattaataccaggtacg

SEQ ID NO: 8 - DNA, 11 bp, synthetic construct
  source 1..11: mol_type = other DNA
cggtatggtat

SEQ ID NO: 9 - DNA, 53 bp, synthetic construct
  source 1..53: mol_type = other DNA
ggcctgctcacgggtcgggcaaacctcacggggctcggtccagtccggtccga

SEQ ID NO: 10 - DNA, 34 bp, synthetic construct
  source 1..34: mol_type = other DNA
tagaccctcgcatccctacaatgccgggacggag